(19) United States
(12) Patent Application Publication
(10) Pub. No.: US 2025/0000872 A1
(43) Pub. Date: Jan. 2, 2025

(54) FORMULATIONS AND DOSING REGIMENS FOR RIP1 KINASE INHIBITORS FOR TREATING AUTOIMMUNE AND INFLAMMATORY DISEASES

(71) Applicants: Eli Lilly and Company, Indianapolis, IN (US); Rigel Pharmaceuticals, Inc., South San Francisco, CA (US)

(72) Inventors: Matthew M. ABERNATHY, Greenfield, IN (US); Tak Cheung Andrew CHOW, Walnut Creek, CA (US); Daniel DAIRAGHI, Carmel, IN (US); William KIELBASA, Orchard Park, NY (US); Esteban MASUDA, Menlo Park, CA (US); David Charles SPERRY, Indianapolis, IN (US); Vanessa Claire TAYLOR, San Francisco, CA (US); Jay L. TUTTLE, San Marcos, CA (US)

(21) Appl. No.: 18/745,814

(22) Filed: Jun. 17, 2024

Related U.S. Application Data
(60) Provisional application No. 63/510,196, filed on Jun. 26, 2023.

(51) Int. Cl. A61K 31/553; A61K 9/16

(52) U.S. Cl. CPC A61K 31/553; A61K 9/1652

(57) ABSTRACT

Disclosed herein are methods of treating a subject having an autoimmune or inflammatory disease, and formulations associated therewith, comprising administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, wherein the compound or the pharmaceutically acceptable salt is administered at a dose of about 12.5 mg to about 125 mg free base equivalent of said compound per dose.

### FIELD

The instant application relates to dosing regimens for receptor-interacting protein-1 kinase (“RIP1”) inhibitor for the treatment of autoimmune and inflammatory diseases, and/or other conditions associated with RIP1 abnormally. In particular, the instant application relates to dosing regimens for Compound 1 for the treatment of autoimmune diseases and inflammatory disorders, such as rheumatoid arthritis, psoriasis, and ulcerative colitis.

### BACKGROUND

Receptor-interacting protein-1 kinase (referred to herein as “RIP1” or “RIPK1” interchangeably) belongs to the tyrosine kinase-like family and is a serine/threonine protein kinase involved in innate immune signaling. RIP1 plays a central role in regulating cell signaling and its role in programmed cell death has been linked to various inflammatory diseases, such as inflammatory bowel disease, psoriasis, and other diseases and/or conditions associated with inflammation and/or necroptotic cell death.

For example, rheumatoid arthritis (RA) is a common, systemic autoimmune inflammatory disease characterized by synovial inflammation leading to pain, swelling, stiffness, and progressive destruction and deformity of small and large joints. Patients experience impaired physical function, social participation, and health-related quality of life. Current expert recommendations for treatment of RA include timely initiation and modification of DMARD therapy to bring patients to a target of sustained low disease activity (LDA) or remission (Fraenkel et al. 2021; Smolen et al. 2022). Achievement of these targets improves short- and long-term patient health outcomes, including prevention of progressive, irreversible structural joint damage (Smolen et al. 2022). The treatment target can be met in most patients with the therapeutic options currently available, which include csDMARDs, bDMARDs, and tsDMARDs. However, 20% to 30% of patients with RA fail to respond to current therapies. For these patients, new treatment options are needed (Smolen et al. 2022).

Likewise, psoriasis is a common, chronic inflammatory skin disease characterized by erythematous, scaly plaques and often associated with other systemic diseases. Although several treatment options are available, a substantial proportion of patients with psoriasis are not receiving treatment or are undertreated because of long-term safety concerns, poor tolerability, failure to achieve or maintain treatment goals, patient preference/tolerability for administration route, cost of medication, and/or access and reimbursement issues (Armstrong et al. 2013).

Currently however, no RIPK1 inhibitor has achieved market authorization, and none appears to have achieved proof of concept. Compound I, (S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, having the follow structure:

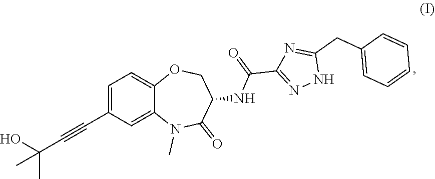 [SMILES: CN1C(=O)[C@@H](NC(=O)c2n[nH]c(Cc3ccccc3)n2)COc2ccc(C#CC(C)(C)O)cc21]

is a novel, potent and selective inhibitor of receptor-interacting protein kinase 1 that blocks inflammatory cell death downstream of receptors such as the TNF receptor and Fas/CD95.

Compound I was previously reported to be an option for potential treatment of autoimmune and inflammatory disorders (WO2021046437, U.S. Pat. No. 11,332,451). Compound I has a linear pharmacokinetics (PK) over a tested dose range up to 1000 mg, and was found to be potent, generally safe, and well tolerated following single and multiple doses. (Yan L. et al. Poster presented at: ASCPT 2021 Annual Meeting; March 2021). Despite this, improved methods of dosing and treatment regimens are still needed for advantageous treatments for autoimmune and inflammatory diseases.

Successful dosing and treatment regimens require careful design of dosing amount, dose frequency, administration route, crystallinity, particle size, excipient, carrier, among others, in order to achieve balanced efficacy and toxicity for a particular indication. It has been reported that the determination of a successful dosing and treatment regimen for a RIPK1 inhibitor is unpredictable.

This unpredictability is best illustrated with the related RIPK1 inhibitor known as GSK2982772, or 5-Benzyl-N-[(3S)-5-methyl-4-oxo-2,3-dihydro-1,5-benzoxazepin-3-yl]-1H-1,2,4-triazole-3-carboxamide, having the following structure:

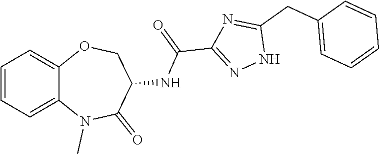 [SMILES: CN1C(=O)[C@@H](NC(=O)c2n[nH]c(Cc3ccccc3)n2)COc2ccccc21]

GSK2982772 was studied in a multicenter, randomized, double-blind, placebo-controlled experimental medicine study in patients with mild-to-moderate active plaque-type psoriasis, under a 60 mg orally b.i.d. as well as a 60 mg orally t.i.d. treatment regimen. The sponsor observed “limited” clinical improvement over placebo, and suggested “higher systemic exposure” may be required. (Weisel et al. Clin Pharmacol Ther. 2020 October; 108 (4): 808-816). The sponsor then evaluated several modified release formulations of GSK2982772 and was able to achieve once per day pharmacokinetic profile (e.g. 80% GSK2982772 was released over 12 h) in the fasted state, although this profile was not maintained with a standard or high-fat meal (Tompson et al. Pharmaceutical Research volume 38, pages 1235-1245 (2021)). The sponsor was then able to overcome the food effect, without sacrificing tolerability, with its proprietary DiffCORE MR and enteric-coated formulations (Tompson et al. Pharmaceutical Research volume 39, pages 153-165 (2022)). Subsequently, one of these modified release formulations was taken to a phase 1 study at 960 mg once daily for up to 84 days. (ClinicalTrials.gov identifier: NCT04316585. Updated Nov. 11, 2021. Accessed Apr. 11, 2023. https://clinicaltrials.gov/ct2/show/NCT04316585).

Meanwhile, GSK2982772 was also investigated in patients with moderate to severe RA under a 60 mg orally b.i.d. as well as a 60 mg orally t.i.d. treatment regimen for up to 84 days. No significant difference was observed over placebo group in terms of disease activity, radiological progression, or inflammatory markers. The sponsor concluded that “inhibition of RIPK1 activity at the GSK2982772 exposure levels evaluated do not translate into meaningful clinical improvement of RA” (Weisel et al., Arthritis Research & Therapy (2021) 23:85). Furthermore, GSK2982772 was investigated in patients with moderate to severe RA under a 60 mg orally t.i.d. treatment regimen for up to 84 days. The sponsor found that “the agent does not lead to differences in measures of histological disease activity or clinical efficacy compared with placebo” and that “RIPK1 may not be a promising therapeutic target in UC when GSK2982772 is used as monotherapy.” (Weisel et al., BMJ Open Gastroenterology 2021; 8: e000680).

GSK2982772 was removed from the sponsor's clinical pipeline as of February 2022, after 9 clinical trials extended over seven years.

### SUMMARY

In several aspects the present disclosure provides therapeutically advantageous formulations, doses, and dosing regimens for Compound I or a pharmaceutically acceptable salt thereof, for the treatment of autoimmune or inflammatory diseases including rheumatoid arthritis, psoriasis, inflammatory bowel disease, ulcerative colitis, psoriatic arthritis, hidradenitis suppurativa, axial sponyloarthritis, Crohn's disease, cutaneous lupus erythematosus, lupus nephritis, systemic lupus erythematosus, cutaneous lupus erythematosus, and atopic dermatitis; or for the treatment of autoimmune or inflammatory diseases that are modulated by receptor-interacting protein (RIP) kinase 1. The doses and dosing regimens include a range of fixed doses and dosing regimens for treating such diseases with efficacy and durability without causing undue toxicity concerns.

The foregoing and other objects and features of the present disclosure will become more apparent from the following detailed description.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Subject” refers generally to mammals and other animals, particularly humans.

“Patient” as used herein refers particularly to humans who are in need of a treatment, for example, of a disease, disorder, or condition described herein.

“Pharmaceutically acceptable excipient” refers to a substance, other than the active ingredient, that is included in a composition comprising the active ingredient. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Excipients can include, but are not limited to, anti-adherents, binders, coatings, enteric coatings, disintegrants, flavorings, sweeteners, colorants, lubricants, glidants, sorbents, preservatives, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

An “adjuvant” is a component that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, aluminum hydroxide, alum, aluminum phosphate, killed bacteria, squalene, detergents, cytokines, paraffin oil, and combination adjuvants, such as Freund's complete adjuvant or Freund's incomplete adjuvant.

“Pharmaceutically acceptable carrier” refers to an excipient that is a carrier or vehicle, such as a suspension aid, solubilizing aid, or aerosolization aid. Remington: The Science and Practice of Pharmacy, The University of the Sciences in Philadelphia, Editor, Lippincott, Williams, & Wilkins, Philadelphia, PA, 21^{st} Edition (2005), incorporated herein by reference, describes exemplary compositions and formulations suitable for pharmaceutical delivery of one or more therapeutic compositions and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

“Pharmaceutically acceptable salt” refers to pharmaceutically acceptable salts of a compound that are derived from a variety of organic and inorganic counter ions as will be known to a person of ordinary skill in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compounds form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as amino acids, formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, tris(hydroxymethyl)aminomethane (Tris), ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.) In particular disclosed embodiments, the compounds may be a formate, trifluoroactate, hydrochloride or sodium salt.

“Solid dispersion” as used herein refers to dispersions of active pharmaceutical ingredient (API) in an inert matrix or in an inert carrier in the solid state. Solid dispersions may be prepared by dissolution of the API together with the matrix or carrier in a solution and subsequently dried. Alternatively solid dispersions may be prepared in any other suitable methods, such as melting, or solvent-melting methods.

“Solvate” refers to a complex formed by combination of solvent molecules with molecules or ions of a solute. The solvent can be an organic solvent, an inorganic solvent, or a mixture of both. Exemplary solvents include, but are not limited to, alcohols, such as methanol, ethanol, propanol; amides such as N,N-dialiphatic amides, such as N,N-dimethylformamide; tetrahydrofuran; alkylsulfoxides, such as dimethylsulfoxide; water; and combinations thereof. The compound described herein can exist in un-solvated as well as solvated forms when combined with solvents, pharmaceutically acceptable or not, such as water, ethanol, and the like. Solvated forms of the presently disclosed compounds are within the scope of the embodiments disclosed herein. When the solvent is water, the solvate is also referred to as a “hydrate”.

“Treating” or “treatment” as used herein concerns treatment of a disease or condition of interest in a patient or subject, particularly a human having the disease or condition of interest, and includes by way of example, and without limitation:
(i) inhibiting the disease or condition, for example, arresting or slowing its development;
(ii) relieving the disease or condition, for example, causing diminution of a symptom or regression of the disease or condition or a symptom thereof; or
(iii) stabilizing the disease or condition.

Moreover, the term “treatment” that appears in the description of a method refers specifically to a treatment according to that particular method.

As used herein, the terms “disease,” “disorder,” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

“Dose” or “dosage amount” as used herein refers to the total amount of Compound I, or a pharmaceutically acceptable salt thereof, that is administered in one single administration.

“Free base equivalent” or “free drug equivalent” when used in conjunction with “dose” herein refers to the dose of free base form of Compound I that may be formed. For example, the Compound I administered at a dose of free base equivalent of Compound I is administered at the dose itself; a solvate of Compound I administered at a dose of free base equivalent of Compound I is administered at a slightly higher dose to adjust for the amount of solvent molecules present which may not contribute to the pharmaceutical efficacy. Likewise, a pharmaceutically acceptable salt of Compound I administered at a dose of free base equivalent of Compound I is administered at a slightly higher dose to adjust for the amount of counterion present which may not contribute to the pharmaceutical efficacy.

“Majority” as used herein refers to an amount that is greater than 50% of the entire amount.

“Primarily” as used herein refers to a probability that is greater than 50%.

“Once per day” “once every day” as used herein refers to administrations every 24 hours. Other terms are similarly construed. For example, “Once every two days” as used herein refers to administrations every 48 hours. “Once every three days” as used herein refers to administrations every 72 hours. “Twice per day” as used herein refers to administrations every 12 hours. “Three times per day” as used herein refers to administrations every 8 hours.

“About” when used in front of a number, for example, a dose amount, refers to +/−0.5 mg. For example, a dose amount of “about 12.5 mg” refers to 12 mg to 13 mg.

### Compounds and Pharmaceutical Compositions

Disclosed herein are dosing regimens for(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or referred to herein as Compound I:

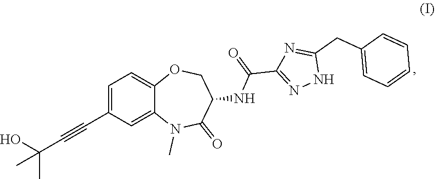 [SMILES: CN1C(=O)[C@@H](NC(=O)c2n[nH]c(Cc3ccccc3)n2)COc2ccc(C#CC(C)(C)O)cc21]

and pharmaceutically acceptable salt thereof in the treatment of autoimmune and inflammatory diseases. Compound I may also be interchangeably referred to as 5-benzyl-N-[(3S)-7-(3-hydroxy-3-methyl-but-1-ynyl)-5-methyl-4-oxo-2,3-dihydro-1,5-benzoxazepin-3-yl]-1H-1,2,4-triazole-3-carboxamide, 5-benzyl-N-[(3S)-7-(3-hydroxy-3-methyl-but-1-ynyl)-4-keto-5-methyl-2,3-dihydro-1,5-benzoxazepin-3-yl]-1H-1,2,4-triazole-3-carboxamide, N-[(3S)-5-methyl-7-(3-methyl-3-oxidanyl-but-1-ynyl)-4-oxidanylidene-2,3-dihydro-1,5-benzoxazepin-3-yl]-5-(phenylmethyl)-1H-1,2,4-triazole-3-carboxamide, 5-benzyl-N-[(3S)-7-(3-hydroxy-3-methylbut-1-ynyl)-5-methyl-4-oxo-2,3-dihydro-1,5-benzoxazepin-3-yl]-1H-1,2,4-triazole-3-carboxamide, or N-[(3S)-7-(3-hydroxy-3-methylbut-1-ynyl)-5-methyl-4-oxo-2,3-dihydro-1,5-benzoxazepin-3-yl]-5-(phenylmethyl)-1H-1,2,4-triazole-3-carboxamide. Compound I may also be referred to as 1H-1,2,4-Triazole-5-carboxamide, 3-(phenylmethyl)-N-[(3S)-2,3,4,5-tetrahydro-7-(3-hydroxy-3-methyl-1-butyn-1-yl)-5-methyl-4-oxo-1,5-benzoxazepin-3-yl]-. The reference to “Compound I” does not encompass salts of Compound I, such as pharmaceutically acceptable salts of Compound I. Compound I is also alternatively and interchangeably referred to as a “free base” or a “free base form” of Compound I. Compound I may be prepared according to the method of Example 2 of WO2019213447, or any other suitable methods.

Although not explicitly described throughout this disclosure, Compound I may exist in tautomeric forms where the hydrogen attached to the triazole ring shifts positions amongst the three triazole nitrogen. The reference to Compound I encompasses these tautomeric forms of Compound I. Also, Compound I or a pharmaceutically acceptable salt thereof may exist in one or more crystalline forms or in the amorphous form. The reference to Compound I encompasses all such forms unless explicitly stated otherwise. Additionally, Compound I or a pharmaceutically acceptable salt thereof may each exist in solvated (e.g. hydrated) or unsolvated forms. The reference to any method, use, composition, kit, or the like, including Compound I also contemplates those including a solvated form of Compound I (in addition to the unsolvated form).

The treatment may use a composition including Compound I or a pharmaceutically acceptable salt thereof, as well as at least one additional component, such as a pharmaceutically acceptable excipient, an adjuvant, or any combination thereof. Pharmaceutically acceptable excipients can be included in pharmaceutical compositions for a variety of purposes, such as to dilute a pharmaceutical composition for delivery to a subject, to facilitate processing of the formulation, to provide advantageous material properties to the formulation, to facilitate dispersion from a delivery device, to stabilize the formulation (e.g., antioxidants or buffers), to provide a pleasant or palatable taste or consistency to the formulation, or the like. The pharmaceutically acceptable excipient(s) may include a pharmaceutically acceptable carrier(s). Exemplary excipients include, but are not limited to: mono-, di-, and polysaccharides, sugar alcohols and other polyols, such as, lactose, glucose, raffinose, melezitose, lactitol, maltitol, trehalose, sucrose, mannitol, starch, or combinations thereof; surfactants, such as sorbitols, diphosphatidyl choline, and lecithin; bulking agents; buffers, such as phosphate and citrate buffers; anti-adherents, such as magnesium stearate; binders, such as saccharides (including disaccharides, such as sucrose and lactose), polysaccharides (such as starches, cellulose, microcrystalline cellulose, cellulose ethers (such as hydroxypropyl cellulose), gelatin, synthetic polymers (such as polyvinylpyrrolidone, polyalkylene glycols); coatings (such as cellulose ethers, including hydroxypropylmethyl cellulose, shellac, corn protein zein, and gelatin); release aids (such as enteric coatings); disintegrants (such as crospovidone, crosslinked sodium carboxymethyl cellulose, and sodium starch glycolate); fillers (such as dibasic calcium phosphate, vegetable fats and oils, lactose, sucrose, glucose, mannitol, sorbitol, calcium carbonate, and magnesium stearate); flavors and sweeteners (such as mint, cherry, anise, peach, apricot or licorice, raspberry, and vanilla; lubricants (such as minerals, exemplified by talc or silica, fats, exemplified by vegetable stearin, magnesium stearate or stearic acid); preservatives (such as antioxidants exemplified by vitamin A, vitamin E, vitamin C, retinyl palmitate, and selenium, amino acids, exemplified by cysteine and methionine, citric acid and sodium citrate, parabens, exemplified by methyl paraben and propyl paraben); colorants; compression aids; emulsifying agents; encapsulation agents; gums; granulation agents; and combinations thereof.

Compound I may be formulated in the pharmaceutical composition per se, or in the form of a pharmaceutically acceptable salt, a tautomer, or a solvate thereof. Typically, such salts are more soluble in aqueous solutions than the corresponding free acids and bases, but salts having lower solubility than the corresponding free acids and bases may also be formed. Pharmaceutical compositions may comprise Compound I or a pharmaceutically acceptable salt thereof at from greater than 0 up to 99% by total weight percent. More typically, pharmaceutical compositions comprising one or more of the disclosed compounds comprise from about 1 to about 20 total weight percent of Compound I or a pharmaceutically acceptable salt thereof, and from about 80 to about 99 weight percent of a pharmaceutically acceptable excipient. In some embodiments, the pharmaceutical composition can further comprise an adjuvant.

Pharmaceutical compositions may be manufactured by any suitable method, such as mixing, dissolving, granulating, dragee-making, levigating, emulsifying, encapsulating, entrapping or lyophilization processes. The pharmaceutical compositions may be formulated using one or more physiologically acceptable excipients (e.g., diluents, carriers, or auxiliaries), one or more adjuvants, or combinations thereof to provide preparations which can be used pharmaceutically.

Suitable pharmaceutical compositions may take a form suitable for virtually any mode of administration, including, for example, topical, ocular, oral, buccal, systemic, nasal, injection, such as i.v. or i.p., transdermal, rectal, vaginal, etc., or a form suitable for administration by inhalation or insufflation. For oral administration, the pharmaceutical compositions may take the form of, for example, lozenges, tablets, or capsules prepared by conventional means with pharmaceutically acceptable excipients, such as: binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); and/or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups, or suspensions, or they may be presented as a dry product for constitution with water or other suitable vehicle before use. Such liquid preparations may be prepared by conventional means with pharmaceutically acceptable excipients such as: suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the active compound, as is well known. Alternatively, other pharmaceutical delivery systems may be employed.

### Diseases, Disorders, and Conditions

Compound I, a pharmaceutically acceptable salt thereof, particularly according to the dosing regimens described here, as well as combinations and/or pharmaceutical compositions thereof, may be used to inhibit a RIP1 kinase by contacting the kinase either in vivo or ex vivo. They may be used to ameliorate or treat a variety of diseases and/or disorders. In particular embodiments, Compound I or a pharmaceutically acceptable salt thereof, particularly according to the dosing regimens described here, combinations and/or pharmaceutical compositions thereof, may be useful for treating conditions in which inhibition of RIP1 or a pathway involving RIP1 is therapeutically useful. In some embodiments, Compound I or a pharmaceutically acceptable salt thereof directly inhibits RIP1 kinase activity. In certain embodiments, Compound I or a pharmaceutically acceptable salt thereof, particularly according to the dosing regimens described here, as well as combinations and/or pharmaceutical compositions thereof, are useful for treating autoimmune diseases or inflammatory diseases.

In certain embodiments, Compound I or a pharmaceutically acceptable salt thereof, particularly according to the dosing regimens described here, as well as combinations and/or pharmaceutical compositions thereof, are useful for treating rheumatoid arthritis, psoriasis, inflammatory bowel disease, ulcerative colitis, psoriatic arthritis, hidradenitis suppurativa, axial sponyloarthritis, or Crohn's disease.

In certain embodiments, Compound I or a pharmaceutically acceptable salt thereof, particularly according to the dosing regimens described here, as well as combinations and/or pharmaceutical compositions thereof, are useful for treating cutaneous lupus erythematosus, lupus nephritis, systemic lupus erythematosus, or atopic dermatitis.

In certain embodiments, Compound I or a pharmaceutically acceptable salt thereof, particularly according to the dosing regimens described here, as well as combinations and/or pharmaceutical compositions thereof, are useful for treating antineutrophil cytoplasmic antibody (ANCA)-associated vasculitis (AAV), OTULIN-related autoinflammatory syndrome (ORAS), A20 haploinsufficiency (HA20), cleavage-resistant RIPK1 induced autoinflammatory (CRIA), NEMO deficiency syndrome, fibrotic diseases, vacuoles, E1 enzyme, X-linked, autoinflammatory and somatic (VEXAS) syndrome, TANK binding kinase 1 (TBK1) and optineurin (OPTN) deficiency disease, familial Amyotrophic Lateral Sclerosis (fALS), or sporadic Amyotrophic Lateral Sclerosis (sALS).

In certain embodiments, Compound I or a pharmaceutically acceptable salt thereof, particularly according to the dosing regimens described here, as well as combinations and/or pharmaceutical compositions thereof, are useful for treating the autoimmune or inflammatory disease that are modulated by receptor-interacting protein (RIP) kinase 1.

As an example, rheumatoid arthritis (RA) typically results in swelling, pain, loss of motion and tenderness of target joints throughout the body. RA is characterized by chronically inflamed synovium that is densely crowded with lymphocytes. The synovial membrane, which is typically one cell layer thick, becomes intensely cellular and assumes a form similar to lymphoid tissue, including dendritic cells, T-, B- and NK cells, macrophages and clusters of plasma cells. This process, as well as a plethora of immunopathological mechanisms including the formation of antigen-immunoglobulin complexes, eventually result in destruction of the integrity of the joint, resulting in deformity, permanent loss of function and/or bone erosion at or near the joint.

Compound I or a pharmaceutically acceptable salt thereof, particularly according to the dosing regimens described here, as well as combinations and/or pharmaceutical compositions thereof, may be used to treat or ameliorate any one, several, or all of these symptoms of RA. Thus, in the context of RA, Compound I or a pharmaceutically acceptable salt thereof, particularly according to the dosing regimens described here, are considered to provide therapeutic benefit when a reduction or amelioration of any of the symptoms commonly associated with RA is achieved, regardless of whether the treatment results in a concomitant treatment of the underlying RA and/or a reduction in the amount of circulating rheumatoid factor (“RF”).

According to American College of Rheumatology (ACR) revised criteria (Hochberg et al. 1992), RA may be classified based on the functional capacity of the patient into four classes. Class I RA patient has “complete functional capacity with ability to carry on all usual duties without handicaps”. Class II RA patient has “functional capacity adequate to conduct normal activities despite handicap of discomfort or limited mobility of one or more joints”. Class III RA patient has “functional capacity adequate to perform only few or none of the duties of usual occupation or of self-care”. Class IV RA patient is “largely or wholly incapacitated” with patient “bedridden or confined to wheelchair, permitting little or no self-care”.

The American College of Rheumatology (ACR) has also developed criteria for defining improvement and clinical remission in RA. Once such parameter, the ACR20 (ACR criteria for 20% clinical improvement), requires a 20% improvement in the tender and swollen joint count, as well as a 20% improvement in 3 of the following 5 parameters: patient's global assessment, physician's global assessment, patient's assessment of pain, degree of disability, and level of acute phase reactant. These criteria have been expanded for 50% and 70% improvement in ACR50 and ACR70, respectively. Other criteria include Paulu's criteria and radiographic progression (e.g. Sharp score).

In some embodiments, therapeutic benefit in patients suffering from RA is achieved when the patient exhibits an ACR20. In specific embodiments, ACR improvements of ACRC50 or even ACR70 may be achieved.

In some embodiments, the subject has moderately-to-severely active RA as defined by the presence of ≥6 swollen joints based on 66 joint count, and ≥6 tender joints based on 68 joint count. It is noted that: (1) The distal interphalangeal joint should be evaluated but not included in the total count to determine eligibility. (2) If a participant has received corticosteroid treatment per Exclusion Criterion [29], the treated joint should be excluded from the joint count.

As another example, psoriasis is a common, chronic inflammatory skin disease characterized by erythematous, scaly plaques and often associated with other systemic diseases. Moderate-to-severe chronic plaque psoriasis are determined by criteria as follows: Plaque psoriasis involving ≥10% body surface area (BSA) and absolute Psoriasis Area and Severity Index (PASI) score ≥12 in affected skin or Static Physician's Global Assessment (sPGA) score of ≥3. Compound I or a pharmaceutically acceptable salt thereof, particularly according to the dosing regimens described here, provide therapeutic benefits to subjects with psoriasis, for example, subjects with moderate-to-severe chronic plaque psoriasis.

As yet another example, ulcerative colitis is a major form of chronic inflammatory bowel disease (IBD). Ulcerative colitis is characterized by abdominal pain and hematochezia (diarrhea with blood). Current available treatments typically seek to control inflammation via anti-inflammatoires and immunosuppressants, although do not correct the underlying cause for the chronic inflammation. Compound I or a pharmaceutically acceptable salt thereof, particularly according to the dosing regimens described here, provide therapeutic benefits to subjects with ulcerative colitis.

### Dosing Regimen

In one aspect, provided herein is a method of treating a subject having an autoimmune or inflammatory disease, comprising administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, wherein the compound or the pharmaceutically acceptable salt is administered at a dose of about 12.5 mg to about 125 mg free base equivalent of said compound per dose. As further described below in, e.g. Examples 1 and 2, the disclosed dose range is advantageous in that it provides a combination of efficacy and tolerability, among other therapeutic factors. For example, with a dose within this range, a therapeutically effective concentration may be maintained during the entire treatment window, particularly at trough between two administrations. Moreover, with a dose within this range, toxicity concern is significantly reduced, as demonstrated by, for example, a sufficient margin of safety.

In some embodiments, the autoimmune or inflammatory disease is a disease modulated by receptor-interacting protein (RIP) kinase 1.

In some embodiments, the autoimmune or inflammatory disease is selected from the group consisting of rheumatoid arthritis, psoriasis, inflammatory bowel disease, ulcerative colitis, psoriatic arthritis, hidradenitis suppurativa, axial sponyloarthritis, Crohn's disease, cutaneous lupus erythematosus, lupus nephritis, systemic lupus erythematosus, atopic dermatitis, antineutrophil cytoplasmic antibody (ANCA)-associated vasculitis (AAV), OTULIN-related autoinflammatory syndrome (ORAS), A20 haploinsufficiency (HA20), cleavage-resistant RIPK1 induced autoinflammatory (CRIA), NEMO deficiency syndrome, fibrotic diseases, vacuoles, E1 enzyme, X-linked, autoinflammatory and somatic (VEXAS) syndrome, TANK binding kinase 1 (TBK1) and optineurin (OPTN) deficiency disease, familial Amyotrophic Lateral Sclerosis (fALS), or sporadic Amyotrophic Lateral Sclerosis (sALS).

In some embodiments, the administering is carried out with a free base form of the compound.

In some embodiments, the administering is carried out with an amorphous form of the compound. As further described below, it was surprisingly discovered that by preparing Compound I in an amorphous state, such as in a solid dispersion, bioavailability is significantly improved over a corresponding preparation with Compound I in a crystalline form. This enables therapeutic efficacy at the lower dose range than otherwise required. Thus, a suitable pharmaceutical composition includes Compound I primarily in the amorphous form. The composition may take the form of an amorphous tablet, such as a solid dispersion tablet. The solid dispersion tablet may be prepared, for example, from a spray dry technique, where a solution of Compound I and a solution of a suitable polymer are mixed and dried to form the solid dispersion. The suitable polymer may assist the dispersion and stabilization of Compound I in the amorphous state. The suitable pharmaceutical composition may take the form of a liquid preparation of Compound I primarily in the amorphous form. For example, the composition may take the form of solid dispersion oral suspension.

In one aspect, provided herein is a method of treating a patient having an autoimmune or inflammatory disease, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 12.5 mg to about 125 mg free base equivalent of said compound per dose.

In some embodiments, the amorphous form of the compound is part of a solid dispersion.

In some embodiments, the solid dispersion is a spray dried dispersion.

In some embodiments, the solid dispersion comprises a polymer a polymer selected from the group consisting of hydroxypropyl methylcellulose acetate succinate (HPMC-AS), polyvinylpyrrolidone/vinyl acetate co-polymer, 6:4 (also referred to as PVP/VA 64 or 6:4 linear random copolymer of N-vinylpyrrolidone and vinyl acetate), Povidone K30 (also referred to as PVP K30, or polyvinylpyrrolidone K30), and hydroxypropyl methylcellulose (HPMC). In some embodiments, a weight ratio (w/w) of free base equivalent of the compound and the polymer is about 1:4 to about 1.2:1.

In some embodiments, the polymer is hydroxypropyl methylcellulose acetate succinate.

In some embodiments, a weight ratio (w/w) of free base equivalent of the compound and the polymer (e.g. hydroxypropyl methylcellulose acetate succinate) is about 1:4 to about 1.2:1. In some embodiments, the weight ratio (w/w) is about 1:3 to about 1:1. The weight ratio in the stated range allows for a balanced dose strength suitable for administration without undue patient compliance difficulties.

In some embodiments, the dose is about 25 mg to about 125 mg free base equivalent of said compound per dose. In some embodiments, the dose is about 25 mg to about 118.75 mg free base equivalent of said compound per dose. In some embodiments, the dose is about 25 mg to about 112.5 mg free base equivalent of said compound per dose. In some embodiments, the dose is about 25 mg to about 106.25 mg free base equivalent of said compound per dose. In some embodiments, the dose is about 25 mg to about 100 mg free base equivalent of said compound per dose. In some embodiments, the dose is about 25 mg to about 93.75 mg free base equivalent of said compound per dose. In some embodiments, the dose is about 25 mg to about 87.5 mg free base equivalent of said compound per dose. In some embodiments, the dose is about 25 mg to about 81.25 mg free base equivalent of said compound per dose. In some embodiments, the dose is about 25 mg to about 75 mg free base equivalent of said compound per dose. In some embodiments, the dose is about 25 mg to about 68.75 mg free base equivalent of said compound per dose. In some embodiments, the dose is about 25 mg to about 62.5 mg free base equivalent of said compound per dose. In some embodiments, the dose is about 25 mg to about 56.25 mg free base equivalent of said compound per dose. In some embodiments, the dose is about 25 mg to about 50 mg free base equivalent of said compound per dose. In some embodiments, the dose is about 25 mg to about 43.75 mg free base equivalent of said compound per dose. In some embodiments, the dose is about 25 mg to about 37.5 mg free base equivalent of said compound per dose. In some embodiments, the dose is about 25 mg to about 31.25 mg free base equivalent of said compound per dose.

In some embodiments, the dose is about 50 mg to about 125 mg free base equivalent of said compound per dose. In some embodiments, the dose is about 50 mg to about 118.75 mg free base equivalent of said compound per dose. In some embodiments, the dose is about 50 mg to about 112.5 mg free base equivalent of said compound per dose. In some embodiments, the dose is about 50 mg to about 106.50 mg free base equivalent of said compound per dose. In some embodiments, the dose is about 50 mg to about 100 mg free base equivalent of said compound per dose. In some embodiments, the dose is about 50 mg to about 93.75 mg free base equivalent of said compound per dose. In some embodiments, the dose is about 50 mg to about 87.5 mg free base equivalent of said compound per dose. In some embodiments, the dose is about 50 mg to about 81.25 mg free base equivalent of said compound per dose. In some embodiments, the dose is about 50 mg to about 75 mg free base equivalent of said compound per dose. In some embodiments, the dose is about 50 mg to about 68.75 mg free base equivalent of said compound per dose. In some embodiments, the dose is about 50 mg to about 62.5 mg free base equivalent of said compound per dose. In some embodiments, the dose is about 50 mg to about 56.50 mg free base equivalent of said compound per dose.

In some embodiments, the dose is about 75 mg to about 125 mg free base equivalent of said compound per dose. In some embodiments, the dose is about 75 mg to about 118.75 mg free base equivalent of said compound per dose. In some embodiments, the dose is about 75 mg to about 112.5 mg free base equivalent of said compound per dose. In some embodiments, the dose is about 75 mg to about 106.75 mg free base equivalent of said compound per dose. In some embodiments, the dose is about 75 mg to about 100 mg free base equivalent of said compound per dose. In some embodiments, the dose is about 75 mg to about 93.75 mg free base equivalent of said compound per dose. In some embodiments, the dose is about 75 mg to about 87.5 mg free base equivalent of said compound per dose. In some embodiments, the dose is about 75 mg to about 81.75 mg free base equivalent of said compound per dose.

In some embodiments, the dose is about 100 mg to about 125 mg free base equivalent of said compound per dose. In some embodiments, the dose is about 100 mg to about 118.75 mg free base equivalent of said compound per dose. In some embodiments, the dose is about 100 mg to about 112.5 mg free base equivalent of said compound per dose. In some embodiments, the dose is about 100 mg to about 106.25 mg free base equivalent of said compound per dose.

In some embodiments, the dose is about 20 mg to about 50 mg free base equivalent of said compound per dose. In some embodiments, the dose is about 20 mg, 25 mg, 30 mg, 35 mg, 40 mg, 45 mg, or 50 mg free base equivalent of said compound per dose. In some embodiments, the dose is about 20 mg to about 30 mg free base equivalent of said compound per dose. In some embodiments, the dose is about 20 mg, 25 mg, or 30 mg free base equivalent of said compound per dose. In some embodiments, the dose is about 45 mg to about 55 mg free base equivalent of said compound per dose. In some embodiments, the dose is about 50 mg to about 70 mg free base equivalent of said compound per dose. In some embodiments, the dose is about 50 mg, 55 mg, 60 mg, 65 mg, or 70 mg free base equivalent of said compound per dose. In some embodiments, the dose is about 70 mg to about 125 mg free base equivalent of said compound per dose. In some embodiments, the dose is about 70 mg, 75 mg, 80 mg, 85 mg, 90 mg, 95 mg, 100 mg, 105 mg, 110 mg, 115 mg, 120 mg, or 125 mg free base equivalent of said compound per dose. In some embodiments, the dose is about 70 mg to about 110 mg free base equivalent of said compound per dose. In some embodiments, the dose is about 70 mg, 75 mg, 80 mg, 85 mg, 90 mg, 95 mg, 100 mg, 105 mg, or 110 mg free base equivalent of said compound per dose. In some embodiments, the dose is about 70 mg to about 90 mg free base equivalent of said compound per dose. In some embodiments, the dose is about 70 mg, 75 mg, 80 mg, 85 mg, or 90 mg free base equivalent of said compound per dose. In some embodiments, the dose is about 90 mg to about 110 mg free base equivalent of said compound per dose. In some embodiments, the dose is about 90 mg, 95 mg, 100 mg, 105 mg, or 110 mg free base equivalent of said compound per dose. In some embodiments, the dose is about 100 mg to about 110 mg free base equivalent of said compound per dose. In some embodiments, the dose is about 100 mg, 105 mg, or 110 mg free base equivalent of said compound per dose. In some embodiments, the dose is about 110 mg to about 125 mg free base equivalent of said compound per dose. In some embodiments, the dose is about 110 mg, 115 mg, 120 mg, or 125 mg free base equivalent of said compound per dose.

In some embodiments, the dose is about 25 mg free base equivalent of said compound per dose. In some embodiments, the dose is about 50 mg free base equivalent of said compound per dose. In some embodiments, the dose is about 75 mg free base equivalent of said compound per dose. In some embodiments, the dose is about 100 mg free base equivalent of said compound per dose. In some embodiments, the dose is about 125 mg free base equivalent of said compound per dose.

In an aspect, provided here is a method of treating a patient having an autoimmune or inflammatory disease, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 25 mg to about 125 mg free base equivalent of said compound per dose.

In some embodiments, the maximum daily dose is about 25 mg to about 125 mg free base equivalent of said compound. In some embodiments, the maximum daily dose is about 25 mg, 50 mg, 75 mg, 100 mg, or 125 mg free base equivalent of said compound.

In some embodiments, with respect to any dose described above, the dose is administered once per day.

In some embodiments, with respect to any dose described above, the dose is administered twice per day. In some embodiments, the dose is administered twice per day and the dose is about 12.5 mg to about 50 mg free base equivalent of said compound per dose. In some embodiments, the dose is administered twice per day and the dose is about 25 mg to about 50 mg free base equivalent of said compound per dose.

In some embodiments, with respect to any dose described above, the dose is administered three times per day. In some embodiments, the dose is administered three times per day and the dose is about 12.5 mg to about 50 mg free base equivalent of said compound per dose. In some embodiments, the dose is administered three times per day and the dose is about 25 mg to about 50 mg free base equivalent of said compound per dose.

In some embodiments, with respect to any dose described above, the dose is administered once every two days.

In some embodiments, with respect to any dose described above, the dose is administered once every three days.

In an aspect, provided herein is a method of treating a patient having an autoimmune or inflammatory disease, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 25 mg to about 125 mg free base equivalent of said compound per dose, and wherein the dose is administered once per day.

In some embodiments, with respect to any dose described above, the dose is administered via oral administration.

In some embodiments, the autoimmune or inflammatory disease is an inflammatory disease.

In some embodiments, the autoimmune or inflammatory disease is an autoimmune disease.

In some embodiments, the autoimmune or inflammatory disease is a tumor necrosis factor (TNF) driven disease.

In some embodiments, the autoimmune or inflammatory disease is rheumatoid arthritis. In some embodiments, the rheumatoid arthritis is Class I, Class II, or Class III rheumatoid arthritis according to ACR revised criteria (Hochberg et al. 1992). In some embodiments, the rheumatoid arthritis is moderately-to-severely active rheumatoid arthritis. In some embodiments, the subject is previously treated with at least one conventional synthetic disease-modifying antirheumatic drug (csDMARD), and either one biologic disease-modifying antirheumatic drug (bDMARD) or one targeted synthetic disease-modifying antirheumatic drug (tsDMARD) treatment.

In some embodiments, with respect to any method above directed to rheumatoid arthritis, the method further comprises administering to the subject one or more oral conventional synthetic disease-modifying antirheumatic drug (csDMARD). In some embodiments, the method further comprises administering to the subject methotrexate (MTX).

In some embodiments, with respect to any method above directed to rheumatoid arthritis, the method further comprises administering to the subject one or more biologic disease-modifying antirheumatic drug (bDMARD). In some embodiments, with respect to any method above directed to rheumatoid arthritis, the method further comprises administering to the subject a tumor necrosis factor (TNF) inhibitor.

In some embodiments, with respect to any method above directed to rheumatoid arthritis, the method further comprises administering to the subject one or more targeted synthetic disease-modifying antirheumatic drug (tsDMARD).

In some embodiments, with respect to any method above directed to rheumatoid arthritis, the subject achieves low disease activity after twelve weeks of treatment, as defined by:
(a) Clinical Disease Activity Index (CDAI) being equal to or less than 10;
(b) Simplified Disease Activity Index (SDAI) being equal to or less than 11;
(c) Disease Activity Score-High-Sensitivity C-Reactive Protein (DAS28-hsCRP) being equal to or less than 3.2; or
(d) Disease Activity Score-Erythrocyte Sedimentation Rate (DAS28-ESR) being equal to or less than 3.2.

In some embodiments, with respect to any method above directed to rheumatoid arthritis, the subject achieves remission after twelve weeks of treatment, as defined by:
(a) Clinical Disease Activity Index (CDAI) being equal to or less than 2.8;
(b) Simplified Disease Activity Index (SDAI) being equal to or less than 3.3;
(c) Disease Activity Score-High-Sensitivity C-Reactive Protein (DAS28-hsCRP) being equal to or less than 2.6;
(d) Disease Activity Score-Erythrocyte Sedimentation Rate (DAS28-ESR) being equal to or less than 2.6.

In some embodiments, with respect to any method above directed to rheumatoid arthritis, the subject achieves ACR70 after twelve weeks of treatment according to American College of Rheumatology criteria.

In some embodiments, with respect to any method above directed to rheumatoid arthritis, the subject achieves ACR50 after twelve weeks of treatment according to American College of Rheumatology criteria.

In some embodiments, with respect to any method above directed to rheumatoid arthritis, the subject achieves ACR20 after twelve weeks of treatment according to American College of Rheumatology criteria.

In some embodiments, with respect to any relevant method above, the autoimmune or inflammatory disease is psoriasis.

In some embodiments, with respect to any relevant method above, the autoimmune or inflammatory disease is plaque psoriasis. In some embodiments, the plaque psoriasis is moderate-to-severe plaque psoriasis, which is defined as plaque psoriasis involving ≥10% body surface area (BSA) and absolute Psoriasis Area and Severity Index (PASI) score ≥12 in affected skin; and/or Static Physician's Global Assessment (sPGA) score of ≥3.

In some embodiments, the patient achieves 75% improvement in Psoriasis Area and Severity Index (PASI) from baseline after twelve weeks of treatment. In some embodiments, the patient achieves 90% improvement in Psoriasis Area and Severity Index (PASI) from baseline after twelve weeks of treatment. In some embodiments, the patient achieves 100% improvement in Psoriasis Area and Severity Index (PASI) from baseline after twelve weeks of treatment. In some embodiments, the patient achieves score 0 (clear) or score 1 (almost clear) on the Static Physician's Global Assessment (sPGA) scale after twelve weeks of treatment.

In some embodiments, the autoimmune or inflammatory disease is inflammatory bowel disease.

In some embodiments, the autoimmune or inflammatory disease is ulcerative colitis.

In some embodiments, the autoimmune or inflammatory disease is Crohn's disease.

In some embodiments, the autoimmune or inflammatory disease is psoriatic arthritis.

In some embodiments, the autoimmune or inflammatory disease is hidradenitis suppurativa.

In some embodiments, the autoimmune or inflammatory disease is axial sponyloarthritis.

In some embodiments, the autoimmune or inflammatory disease is one not driven by tumor necrosis factor (TNF).

In some embodiments, the autoimmune or inflammatory disease is the autoimmune or inflammatory disease is cutaneous lupus erythematosus.

In some embodiments, the autoimmune or inflammatory disease is lupus nephritis.

In some embodiments, the autoimmune or inflammatory disease is systemic lupus erythematosus.

In some embodiments, the autoimmune or inflammatory disease is cutaneous lupus erythematosus.

In some embodiments, the autoimmune or inflammatory disease is atopic dermatitis.

In some embodiments, the autoimmune or inflammatory disease is OTULIN-related autoinflammatory syndrome (ORAS).

In some embodiments, the autoimmune or inflammatory disease is A20 haploinsufficiency (HA20). A20 is also known as TNF-α-induced protein 3 (TNFAIP3)

In some embodiments, the autoimmune or inflammatory disease is cleavage-resistant RIPK1 induced autoinflammatory (CRIA).

In some embodiments, the autoimmune or inflammatory disease is NEMO deficiency syndrome.

In some embodiments, the autoimmune or inflammatory disease is antineutrophil cytoplasmic antibody (ANCA)-associated vasculitis (AAV).

In some embodiments, the autoimmune or inflammatory disease is a fibrotic disease.

In some embodiments, the autoimmune or inflammatory disease is a vacuoles, E1 enzyme, X-linked, autoinflammatory and somatic (VEXAS) syndrome.

In some embodiments, the autoimmune or inflammatory disease is a TANK binding kinase 1 (TBK1)/optineurin (OPTN) deficiency disease.

In some embodiments, the autoimmune or inflammatory disease is familial Amyotrophic Lateral Sclerosis (fALS), or sporadic Amyotrophic Lateral Sclerosis (sALS).

In one aspect, provided herein is a method of treating a patient having rheumatoid arthritis, comprising administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In one aspect, provided herein is a method of treating a patient having rheumatoid arthritis, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 25 mg to about 125 mg per dose, once per day.

In one aspect, provided herein is a method of treating a patient having psoriasis, comprising administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In one aspect, provided herein is a method of treating a patient having psoriasis, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 25 mg to about 125 mg per dose, once per day.

In one aspect, provided herein is a method of treating a patient having ulcerative colitis, comprising administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In one aspect, provided herein is a method of treating a patient having ulcerative colitis, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In one aspect, provided herein is a pharmaceutical composition, comprising a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, and a pharmaceutically acceptable diluent or carrier, wherein the composition comprises about 12.5 mg to about 125 mg per dose of free base equivalent of the compound, and wherein the composition is administered to a patient having an autoimmune or inflammatory disease, and wherein the administering comprises administering to the patient the compound, or a pharmaceutically acceptable salt thereof, according to any above aspects and embodiments.

In one aspect, provided herein is a pharmaceutical composition for use in the treatment of an autoimmune or inflammatory disease in a patient, comprising a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, wherein the composition comprises about 12.5 mg to about 125 mg per dose of free base equivalent of the compound. In some embodiments, the treatment is according to the method of any above aspects and embodiments.

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, and (2) a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC).

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, and (2) a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC).

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, and (2) a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC).

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, and (2) a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC), wherein a weight ratio (w/w) of the compound to the polymer is about 1:4 to about 1.2:1.

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, and (2) hydroxypropyl methylcellulose acetate succinate (HPMC-AS), wherein a weight ratio (w/w) of the compound to the HPMC-AS is about 1:4 to about 1.2:1.

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, and (2) hydroxypropyl methylcellulose acetate succinate (HPMC-AS), wherein a weight ratio (w/w) of the compound to the HPMC-AS is about 1:3 to about 1:1.

In one aspect, provided herein is a pharmaceutical composition comprising (1) a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, and (2) a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC), wherein the composition comprises about 12.5 mg to about 125 mg per dose of free base equivalent of the compound, and wherein a weight ratio (w/w) of free base equivalent of the compound to the polymer is about 1:4 to about 1.2:1.

In some embodiments, the polymer comprises hydroxypropyl methylcellulose acetate succinate (HPMC-AS). In some embodiments, a weight ratio (w/w) of free base equivalent of the compound and the hydroxypropyl methylcellulose acetate succinate is about 1:3 to about 1:1.

In some embodiments, and the composition further comprises a pharmaceutically acceptable diluent or carrier.

In one aspect, provided here is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, for use in the treatment of an autoimmune or inflammatory disease in a patient, wherein the compound or the pharmaceutically acceptable salt is administered at a dose of about 12.5 mg to about 125 mg free base equivalent of said compound per dose. In some embodiments, the treatment is according to the method of any above aspects and embodiments.

In one aspect, provided here is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, for use in the treatment of an autoimmune or inflammatory disease in a patient, wherein the compound is administered at a dose of about 12.5 mg to about 125 mg free base equivalent of said compound per dose. In some embodiments, the treatment is according to the method of any above aspects and embodiments.

In one aspect, provided here is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, for use in the treatment of an autoimmune or inflammatory disease in a patient, wherein the compound is administered in an amorphous state at a dose of about 12.5 mg to about 125 mg free base equivalent of said compound per dose. In some embodiments, the treatment is according to the method of any above aspects and embodiments.

In one aspect, provided here is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, for use in the treatment of rheumatoid arthritis in a patient, wherein the compound is administered in an amorphous state at a dose of about 25 mg to about 125 mg free base equivalent of said compound per dose. In some embodiments, the treatment is according to the method of any above aspects and embodiments.

In one aspect, provided here is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, for use in the treatment of psoriasis in a patient, wherein the compound is administered in an amorphous state at a dose of about 25 mg to about 125 mg free base equivalent of said compound per dose. In some embodiments, the treatment is according to the method of any above aspects and embodiments.

In one aspect, provided here is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, for use in the treatment of ulcerative colitis in a patient, wherein the compound is administered in an amorphous state at a dose of about 12.5 mg to about 125 mg free base equivalent of said compound per dose. In some embodiments, the treatment is according to the method of any above aspects and embodiments.

In one aspect, provided here is use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the compound or the pharmaceutically acceptable salt is administered at a dose of about 12.5 mg to about 125 mg free base equivalent of said compound per dose. In some embodiments, the treatment is according to the method of any above aspects and embodiments.

In one aspect, provided here is use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, in the manufacture of a medicament for the treatment of an autoimmune or inflammatory disease in a patient, wherein the compound is administered at a dose of about 12.5 mg to about 125 mg free base equivalent of said compound per dose. In some embodiments, the treatment is according to the method of any above aspects and embodiments.

In one aspect, provided here is use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, in the manufacture of a medicament for the treatment of an autoimmune or inflammatory disease in a patient, wherein the compound is administered in an amorphous state at a dose of about 12.5 mg to about 125 mg free base equivalent of said compound per dose. In some embodiments, the treatment is according to the method of any above aspects and embodiments.

In one aspect, provided here is use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, in the manufacture of a medicament for the treatment of rheumatoid arthritis in a patient, wherein the compound is administered in an amorphous state at a dose of about 25 mg to about 125 mg free base equivalent of said compound per dose. In some embodiments, the treatment is according to the method of any above aspects and embodiments.

In one aspect, provided here is use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, in the manufacture of a medicament for the treatment of psoriasis in a patient, wherein the compound is administered in an amorphous state at a dose of about 25 mg to about 125 mg free base equivalent of said compound per dose. In some embodiments, the treatment is according to the method of any above aspects and embodiments.

In one aspect, provided here is use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, in the manufacture of a medicament for the treatment of ulcerative colitis in a patient, wherein the compound is administered in an amorphous state at a dose of about 12.5 mg to about 125 mg free base equivalent of said compound per dose. In some embodiments, the treatment is according to the method of any above aspects and embodiments.

In one aspect, provided herein is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, for use in the manufacture of a medicament for treatment of an autoimmune or inflammatory disease in a patient, wherein the medicament comprises a free base equivalent of the compound at a strength of about 12.5 mg per dose unit. In some embodiments, the treatment is according to the method of any above aspects and embodiments.

In one aspect, provided herein is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, for use in the manufacture of a medicament for treatment of an autoimmune or inflammatory disease in a patient, wherein the medicament comprises a free base equivalent of the compound at a strength of about 25 mg per dose unit. In some embodiments, the treatment is according to the method of any above aspects and embodiments.

In one aspect, provided herein is compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, for use in the manufacture of a medicament for treatment of an autoimmune or inflammatory disease in a patient, wherein the medicament comprises a free base equivalent of the compound at a strength of about 62.5 mg per dose unit. In some embodiments, the treatment is according to the method of any above aspects and embodiments.

In one aspect, provided here is a use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the medicament comprises a free base equivalent of compound at a strength of about 12.5 mg per dose unit. In some embodiments, the treatment is according to the method of any above aspects and embodiments.

In one aspect, provided here is a use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the medicament comprises a free base equivalent of the compound at a strength of about 25 mg per dose unit. In some embodiments, the treatment is according to the method of any above aspects and embodiments.

In one aspect, provided here is a use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the medicament comprises a free base equivalent of the compound at a strength of about 62.5 mg per dose unit. In some embodiments, the treatment is according to the method of any above aspects and embodiments.

In one aspect, provided here is a use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the medicament comprises a free base equivalent of compound at a strength of about 12.5 mg per dose unit. In some embodiments, the treatment is according to the method of any above aspects and embodiments.

In one aspect, provided here is a use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the medicament comprises a free base equivalent of the compound at a strength of about 25 mg per dose unit. In some embodiments, the treatment is according to the method of any above aspects and embodiments.

In one aspect, provided here is a use of compound(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide or a pharmaceutically acceptable salt thereof, in the manufacture of a medicament for treatment of an autoimmune or inflammatory disease in a patient, wherein the medicament comprises a free base equivalent of the compound at a strength of about 62.5 mg per dose unit. In some embodiments, the treatment is according to the method of any above aspects and embodiments.

In some embodiments, the autoimmune or inflammatory disease is a disease modulated by receptor-interacting protein (RIP) kinase 1.

In some embodiments, the autoimmune or inflammatory disease is selected from the group consisting of rheumatoid arthritis, psoriasis, inflammatory bowel disease, ulcerative colitis, psoriatic arthritis, hidradenitis suppurativa, axial sponyloarthritis, Crohn's disease, cutaneous lupus erythematosus, lupus nephritis, systemic lupus erythematosus, atopic dermatitis, antineutrophil cytoplasmic antibody (ANCA)-associated vasculitis (AAV), OTULIN-related autoinflammatory syndrome (ORAS), A20 haploinsufficiency (HA20), cleavage-resistant RIPK1 induced autoinflammatory (CRIA), NEMO deficiency syndrome, fibrotic diseases, vacuoles, E1 enzyme, X-linked, autoinflammatory and somatic (VEXAS) syndrome, TANK binding kinase 1 (TBK1) and optineurin (OPTN) deficiency disease, familial Amyotrophic Lateral Sclerosis (fALS), or sporadic Amyotrophic Lateral Sclerosis (sALS).

In some embodiments, the treatment uses a free base form of the compound.

In some embodiments, the treatment is carried out with an amorphous form of the compound.

In some embodiments, the treatment is of a patient having an autoimmune or inflammatory disease, and comprises administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 12.5 mg to about 125 mg free base equivalent of said compound per dose.

In some embodiments, the amorphous form of the compound is part of a solid dispersion.

In some embodiments, the solid dispersion is a spray dried dispersion.

In some embodiments, the solid dispersion comprises a polymer a polymer selected from the group consisting of hydroxypropyl methylcellulose acetate succinate (HPMC-AS), polyvinylpyrrolidone/vinyl acetate co-polymer, 6:4 (also referred to as PVP/VA 64 or 6:4 linear random copolymer of N-vinylpyrrolidone and vinyl acetate), Povidone K30 (also referred to as PVP K30, or polyvinylpyrrolidone K30), and hydroxypropyl methylcellulose (HPMC). In some embodiments, a weight ratio (w/w) of free base equivalent of the compound and the polymer is about 1:4 to about 1.2:1.

In some embodiments, the polymer is hydroxypropyl methylcellulose acetate succinate.

In some embodiments, a weight ratio (w/w) of free base equivalent of the compound and the polymer (e.g. hydroxypropyl methylcellulose acetate succinate) is about 1:4 to about 1.2:1. In some embodiments, the weight ratio (w/w) is about 1:3 to about 1:1. The weight ratio in the stated range allows for a balanced dose strength suitable for administration without undue patient compliance difficulties.

In some embodiments, the dose is about 25 mg to about 125 mg free base equivalent of said compound per dose. In some embodiments, the dose is about 25 mg to about 118.75 mg free base equivalent of said compound per dose. In some embodiments, the dose is about 25 mg to about 112.5 mg free base equivalent of said compound per dose. In some embodiments, the dose is about 25 mg to about 106.25 mg free base equivalent of said compound per dose. In some embodiments, the dose is about 25 mg to about 100 mg free base equivalent of said compound per dose. In some embodiments, the dose is about 25 mg to about 93.75 mg free base equivalent of said compound per dose. In some embodiments, the dose is about 25 mg to about 87.5 mg free base equivalent of said compound per dose. In some embodiments, the dose is about 25 mg to about 81.25 mg free base equivalent of said compound per dose. In some embodiments, the dose is about 25 mg to about 75 mg free base equivalent of said compound per dose. In some embodiments, the dose is about 25 mg to about 68.75 mg free base equivalent of said compound per dose. In some embodiments, the dose is about 25 mg to about 62.5 mg free base equivalent of said compound per dose. In some embodiments, the dose is about 25 mg to about 56.25 mg free base equivalent of said compound per dose. In some embodiments, the dose is about 25 mg to about 50 mg free base equivalent of said compound per dose. In some embodiments, the dose is about 25 mg to about 43.75 mg free base equivalent of said compound per dose. In some embodiments, the dose is about 25 mg to about 37.5 mg free base equivalent of said compound per dose. In some embodiments, the dose is about 25 mg to about 31.25 mg free base equivalent of said compound per dose.

In some embodiments, the dose is about 50 mg to about 125 mg free base equivalent of said compound per dose. In some embodiments, the dose is about 50 mg to about 118.75 mg free base equivalent of said compound per dose. In some embodiments, the dose is about 50 mg to about 112.5 mg free base equivalent of said compound per dose. In some embodiments, the dose is about 50 mg to about 106.50 mg free base equivalent of said compound per dose. In some embodiments, the dose is about 50 mg to about 100 mg free base equivalent of said compound per dose. In some embodiments, the dose is about 50 mg to about 93.75 mg free base equivalent of said compound per dose. In some embodiments, the dose is about 50 mg to about 87.5 mg free base equivalent of said compound per dose. In some embodiments, the dose is about 50 mg to about 81.25 mg free base equivalent of said compound per dose. In some embodiments, the dose is about 50 mg to about 75 mg free base equivalent of said compound per dose. In some embodiments, the dose is about 50 mg to about 68.75 mg free base equivalent of said compound per dose. In some embodiments, the dose is about 50 mg to about 62.5 mg free base equivalent of said compound per dose. In some embodiments, the dose is about 50 mg to about 56.50 mg free base equivalent of said compound per dose.

In some embodiments, the dose is about 75 mg to about 125 mg free base equivalent of said compound per dose. In some embodiments, the dose is about 75 mg to about 118.75 mg free base equivalent of said compound per dose. In some embodiments, the dose is about 75 mg to about 112.5 mg free base equivalent of said compound per dose. In some embodiments, the dose is about 75 mg to about 106.75 mg free base equivalent of said compound per dose. In some embodiments, the dose is about 75 mg to about 100 mg free base equivalent of said compound per dose. In some embodiments, the dose is about 75 mg to about 93.75 mg free base equivalent of said compound per dose. In some embodiments, the dose is about 75 mg to about 87.5 mg free base equivalent of said compound per dose. In some embodiments, the dose is about 75 mg to about 81.75 mg free base equivalent of said compound per dose.

In some embodiments, the dose is about 100 mg to about 125 mg free base equivalent of said compound per dose. In some embodiments, the dose is about 100 mg to about 118.75 mg free base equivalent of said compound per dose. In some embodiments, the dose is about 100 mg to about 112.5 mg free base equivalent of said compound per dose. In some embodiments, the dose is about 100 mg to about 106.25 mg free base equivalent of said compound per dose.

In some embodiments, the dose is about 20 mg to about 50 mg free base equivalent of said compound per dose. In some embodiments, the dose is about 20 mg, 25 mg, 30 mg, 35 mg, 40 mg, 45 mg, or 50 mg free base equivalent of said compound per dose. In some embodiments, the dose is about 20 mg to about 30 mg free base equivalent of said compound per dose. In some embodiments, the dose is about 20 mg, 25 mg, or 30 mg free base equivalent of said compound per dose. In some embodiments, the dose is about 45 mg to about 55 mg free base equivalent of said compound per dose. In some embodiments, the dose is about 50 mg to about 70 mg free base equivalent of said compound per dose. In some embodiments, the dose is about 50 mg, 55 mg, 60 mg, 65 mg, or 70 mg free base equivalent of said compound per dose. In some embodiments, the dose is about 70 mg to about 125 mg free base equivalent of said compound per dose. In some embodiments, the dose is about 70 mg, 75 mg, 80 mg, 85 mg, 90 mg, 95 mg, 100 mg, 105 mg, 110 mg, 115 mg, 120 mg, or 125 mg free base equivalent of said compound per dose. In some embodiments, the dose is about 70 mg to about 110 mg free base equivalent of said compound per dose. In some embodiments, the dose is about 70 mg, 75 mg, 80 mg, 85 mg, 90 mg, 95 mg, 100 mg, 105 mg, or 110 mg free base equivalent of said compound per dose. In some embodiments, the dose is about 70 mg to about 90 mg free base equivalent of said compound per dose. In some embodiments, the dose is about 70 mg, 75 mg, 80 mg, 85 mg, or 90 mg free base equivalent of said compound per dose. In some embodiments, the dose is about 90 mg to about 110 mg free base equivalent of said compound per dose. In some embodiments, the dose is about 90 mg, 95 mg, 100 mg, 105 mg, or 110 mg free base equivalent of said compound per dose. In some embodiments, the dose is about 100 mg to about 110 mg free base equivalent of said compound per dose. In some embodiments, the dose is about 100 mg, 105 mg, or 110 mg free base equivalent of said compound per dose. In some embodiments, the dose is about 110 mg to about 125 mg free base equivalent of said compound per dose. In some embodiments, the dose is about 110 mg, 115 mg, 120 mg, or 125 mg free base equivalent of said compound per dose.

In some embodiments, the dose is about 25 mg free base equivalent of said compound per dose. In some embodiments, the dose is about 50 mg free base equivalent of said compound per dose. In some embodiments, the dose is about 75 mg free base equivalent of said compound per dose. In some embodiments, the dose is about 100 mg free base equivalent of said compound per dose. In some embodiments, the dose is about 125 mg free base equivalent of said compound per dose.

In some embodiments, the treatment is of a patient having an autoimmune or inflammatory disease, and comprises administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 25 mg to about 125 mg free base equivalent of said compound per dose.

In some embodiments, the maximum daily dose is about 25 mg to about 125 mg free base equivalent of said compound. In some embodiments, the maximum daily dose is about 25 mg, 50 mg, 75 mg, 100 mg, or 125 mg free base equivalent of said compound.

In some embodiments, with respect to any dose described above, the dose is administered once per day.

In some embodiments, with respect to any dose described above, the dose is administered twice per day. In some embodiments, the dose is administered twice per day and the dose is about 12.5 mg to about 50 mg free base equivalent of said compound per dose. In some embodiments, the dose is administered twice per day and the dose is about 25 mg to about 50 mg free base equivalent of said compound per dose.

In some embodiments, with respect to any dose described above, the dose is administered three times per day. In some embodiments, the dose is administered three times per day and the dose is about 12.5 mg to about 50 mg free base equivalent of said compound per dose. In some embodiments, the dose is administered three times per day and the dose is about 25 mg to about 50 mg free base equivalent of said compound per dose.

In some embodiments, with respect to any dose described above, the dose is administered once every two days.

In some embodiments, with respect to any dose described above, the dose is administered once every three days.

In some embodiments, the treatment is of a patient having an autoimmune or inflammatory disease, and comprises administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 25 mg to about 125 mg free base equivalent of said compound per dose, and wherein the dose is administered once per day.

In some embodiments, with respect to any dose described above, the dose is administered via oral administration.

In some embodiments, the autoimmune or inflammatory disease is an inflammatory disease.

In some embodiments, the autoimmune or inflammatory disease is an autoimmune disease.

In some embodiments, the autoimmune or inflammatory disease is a tumor necrosis factor (TNF) driven disease.

In some embodiments, the autoimmune or inflammatory disease is rheumatoid arthritis. In some embodiments, the rheumatoid arthritis is Class I, Class II, or Class III rheumatoid arthritis according to ACR revised criteria (Hochberg et al. 1992). In some embodiments, the rheumatoid arthritis is moderately-to-severely active rheumatoid arthritis. In some embodiments, the subject is previously treated with at least one conventional synthetic disease-modifying antirheumatic drug (csDMARD), and either one biologic disease-modifying antirheumatic drug (bDMARD) or one targeted synthetic disease-modifying antirheumatic drug (tsDMARD) treatment.

In some embodiments, with respect to rheumatoid arthritis, the treatment further comprises administering to the subject one or more oral conventional synthetic disease-modifying antirheumatic drug (csDMARD). In some embodiments, the treatment further comprises administering to the subject methotrexate (MTX).

In some embodiments, with respect to rheumatoid arthritis, the treatment further comprises administering to the subject one or more biologic disease-modifying antirheumatic drug (bDMARD). In some embodiments, with respect to rheumatoid arthritis, the treatment further comprises administering to the subject a tumor necrosis factor (TNF) inhibitor.

In some embodiments, with respect to rheumatoid arthritis, the treatment further comprises administering to the subject one or more targeted synthetic disease-modifying antirheumatic drug (tsDMARD).

In some embodiments, with respect to rheumatoid arthritis, the subject achieves low disease activity after twelve weeks of treatment, as defined by:
(a) Clinical Disease Activity Index (CDAI) being equal to or less than 10;
(b) Simplified Disease Activity Index (SDAI) being equal to or less than 11;
(c) Disease Activity Score-High-Sensitivity C-Reactive Protein (DAS28-hsCRP) being equal to or less than 3.2; or
(d) Disease Activity Score-Erythrocyte Sedimentation Rate (DAS28-ESR) being equal to or less than 3.2.

In some embodiments, with respect to rheumatoid arthritis, the subject achieves remission after twelve weeks of treatment, as defined by:
(a) Clinical Disease Activity Index (CDAI) being equal to or less than 2.8;
(b) Simplified Disease Activity Index (SDAI) being equal to or less than 3.3;
(c) Disease Activity Score-High-Sensitivity C-Reactive Protein (DAS28-hsCRP) being equal to or less than 2.6;
(d) Disease Activity Score-Erythrocyte Sedimentation Rate (DAS28-ESR) being equal to or less than 2.6.

In some embodiments, with respect to rheumatoid arthritis, the subject achieves ACR70 after twelve weeks of treatment according to American College of Rheumatology criteria.

In some embodiments, with respect to rheumatoid arthritis, the subject achieves ACR50 after twelve weeks of treatment according to American College of Rheumatology criteria.

In some embodiments, with respect to rheumatoid arthritis, the subject achieves ACR20 after twelve weeks of treatment according to American College of Rheumatology criteria.

In some embodiments, the autoimmune or inflammatory disease is psoriasis.

In some embodiments, the autoimmune or inflammatory disease is plaque psoriasis. In some embodiments, the plaque psoriasis is moderate-to-severe plaque psoriasis, which is defined as plaque psoriasis involving ≥10% body surface area (BSA) and absolute Psoriasis Area and Severity Index (PASI) score ≥12 in affected skin; and/or Static Physician's Global Assessment (sPGA) score of ≥3.

In some embodiments, the patient achieves 75% improvement in Psoriasis Area and Severity Index (PASI) from baseline after twelve weeks of treatment. In some embodiments, the patient achieves 90% improvement in Psoriasis Area and Severity Index (PASI) from baseline after twelve weeks of treatment. In some embodiments, the patient achieves 100% improvement in Psoriasis Area and Severity Index (PASI) from baseline after twelve weeks of treatment. In some embodiments, the patient achieves score 0 (clear) or score 1 (almost clear) on the Static Physician's Global Assessment (sPGA) scale after twelve weeks of treatment.

In some embodiments, the autoimmune or inflammatory disease is inflammatory bowel disease.

In some embodiments, the autoimmune or inflammatory disease is ulcerative colitis.

In some embodiments, the autoimmune or inflammatory disease is Crohn's disease.

In some embodiments, the autoimmune or inflammatory disease is psoriatic arthritis.

In some embodiments, the autoimmune or inflammatory disease is hidradenitis suppurativa.

In some embodiments, the autoimmune or inflammatory disease is axial sponyloarthritis.

In some embodiments, the autoimmune or inflammatory disease is one not driven by tumor necrosis factor (TNF).

In some embodiments, the autoimmune or inflammatory disease is the autoimmune or inflammatory disease is cutaneous lupus erythematosus.

In some embodiments, the autoimmune or inflammatory disease is lupus nephritis.

In some embodiments, the autoimmune or inflammatory disease is systemic lupus erythematosus.

In some embodiments, the autoimmune or inflammatory disease is cutaneous lupus erythematosus.

In some embodiments, the autoimmune or inflammatory disease is atopic dermatitis.

In some embodiments, the autoimmune or inflammatory disease is OTULIN-related autoinflammatory syndrome (ORAS).

In some embodiments, the autoimmune or inflammatory disease is A20 haploinsufficiency (HA20). A20 is also known as TNF-α-induced protein 3 (TNFAIP3)

In some embodiments, the autoimmune or inflammatory disease is cleavage-resistant RIPK1 induced autoinflammatory (CRIA).

In some embodiments, the autoimmune or inflammatory disease is NEMO deficiency syndrome.

In some embodiments, the autoimmune or inflammatory disease is antineutrophil cytoplasmic antibody (ANCA)-associated vasculitis (AAV).

In some embodiments, the autoimmune or inflammatory disease is a fibrotic disease.

In some embodiments, the autoimmune or inflammatory disease is a vacuoles, E1 enzyme, X-linked, autoinflammatory and somatic (VEXAS) syndrome.

In some embodiments, the autoimmune or inflammatory disease is a TANK binding kinase 1 (TBK1)/optineurin (OPTN) deficiency disease.

In some embodiments, the autoimmune or inflammatory disease is familial Amyotrophic Lateral Sclerosis (fALS), or sporadic Amyotrophic Lateral Sclerosis (sALS).

In some embodiments, the treatment is of a patient having rheumatoid arthritis, and comprises administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In some embodiments, the treatment is of a patient having rheumatoid arthritis, and comprises administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 25 mg to about 125 mg per dose, once per day.

In some embodiments, the treatment is according to a method of treating a patient having psoriasis, and comprises administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In some embodiments, the treatment is according to a method of treating a patient having psoriasis, and comprises administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 25 mg to about 125 mg per dose, once per day.

In some embodiments, the treatment is according to a method of treating a patient having ulcerative colitis, and comprises administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In some embodiments, the treatment is of according to method of treating a patient having ulcerative colitis, and comprises administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound is administered at a dose of about 12.5 mg to about 125 mg per dose, once per day.

In one aspect, provided here is a pharmaceutical unit dosage composition, comprising about 12.5 mg to about 125 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide.

In one aspect, provided here is a pharmaceutical unit dosage composition, comprising about 25 mg to about 125 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide.

In one aspect, provided here is a pharmaceutical unit dosage composition, comprising about 12.5 mg, 25 mg, 37.5 mg, 50 mg, 62.5 mg, 75 mg, 87.5 mg, 100 mg, 112.5 mg, or 125 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide.

In one aspect, provided here is a pharmaceutical unit dosage composition, comprising about 12.5 mg to about 125 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, said unit dosage composition suitable for oral administration.

In one aspect, provided here is a pharmaceutical unit dosage composition, comprising about 12.5 mg, 25 mg, 37.5 mg, 50 mg, 62.5 mg, 75 mg, 87.5 mg, 100 mg, 112.5 mg, or 125 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, said unit dosage composition suitable for oral administration.

In one aspect, provided here is a pharmaceutical unit dosage composition, comprising about 25 mg to about 125 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, said unit dosage composition suitable for oral administration.

In some embodiments, the pharmaceutical unit dosage composition, comprising about 12.5 mg, 25 mg, 37.5 mg, 50 mg, 62.5 mg, or 75 mg of the compound.

In one aspect, provided here is a pharmaceutical unit dosage composition, comprising about 12.5 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, said unit dosage composition suitable for oral administration.

In one aspect, provided here is a pharmaceutical unit dosage composition, comprising about 25 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, said unit dosage composition suitable for oral administration

In one aspect, provided here is a pharmaceutical unit dosage composition, comprising about 62.5 mg of a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide an amorphous form, said unit dosage composition suitable for oral administration.

In some embodiments, the pharmaceutical unit dosage composition is used according to any method or use described above.

### Example 1

Compound I was studied in a Phase I, single-center study to investigate the safety, tolerability, and PK of single and multiple doses of Compound I in healthy volunteers. The study was conducted in 3 parts: single-ascending doses (Part 1A), multiple-ascending doses (Part 2), and formulation assessment (Part 1B and Part 3). Dosing regimen administered in the study is shown in Table 1:

TABLE 1
Compound I Dosing Regimen in First-in-Human Study

| | | | | Investigational | | Route of | |
|---|---|---|---|---|---|---|---|
| | | | | Medicinal | Dose Level | Administration, | Dosing |
| Part | Cohort | Period | Regimen | Product | (mg)ª | Prandial State | Frequency |
| 1A | 1 | NA | A | LIPID or matching | 12 | Oral, fasted | Once |
| | 2 | | B | placebo^{b} | 60 | | |
| | 3 | | C | | 180 | | |
| | 4 | | D | ALT1 suspension | 300 | | |
| | 5 | | E | or matching | 750 | | |
| | 6 | | F | placebo^{b,c} | 1000 | | |
| 1B | 1 | | I | ALT1 suspension^{b,c} | 180 | | |
| | 2 | | J | ALT2 suspension^{b,c} | 180 | | |
| 2 | 1 | | K | ALT1 suspension | 180 | | QD for |
| | 2 | | L | or matching | 500 | | 14 days |
| | | | | placebo^{b,c} | | | |
| 3 | NA | 1, 2, and 3 | P | ALT1 suspension^{b,c} | 125 | | Once |
| | | (randomized) | Q | ALT1 tablet | 125 | | |
| | | | R | | 125 | Oral, fed | |
| | | | | | | (high-fat | |
| | | | | | | breakfast^{d}) | |

Abbreviations: ALT = alternative; IMP = investigational medicinal product; LIPID = lipid oral solution; NA = not applicable; QD = once daily.
IMP guide: ALT1 suspension = Compound I alternative formulation 1 administered as an oral suspension; ALT2 suspension = Compound I alternative formulation 2 administered as an oral suspension; LIPID = Compound I lipid oral solution, free drug equivalent.
^{a}Free drug equivalent.
^{b}A Listerine strip was administered immediately before the LIPID, ALT1 suspension, and ALT2 suspension formulations to mask the taste.
^{c}ALT1 and ALT2 were spray-dried dispersion powders for oral suspension, administered as oral suspensions (ALT1 suspension and ALT2 suspension, respectively) according to the study-specific dosing instructions.
^{d}The high fat breakfast comprised 1 hash brown, 2 rashers of Sainsbury's streak bacon (grilled), 1 small (45 g) egg fried in 10 g of butter, 2 slices of white medium sliced bread with 20 g of butter, and 240 mL full-fat milk, in line with FDA guidance.

Exposure to Compound I increased in a proportional manner across the dose range of 12 to 1000 mg. The highest Compound I exposure in this study was observed following administration of a single dose of 1000 mg ALT1 suspension, with geometric mean AUC_{0-∞} and Cₘₐₓ values of 499 000 ng×h/mL and 26 000 ng/mL, respectively, which was below the exposure at NOAEL 1000 mg/kg/day from monkey 28-day GLP toxicity study.

Exposure to Compound I was determined following 180 mg QD and 500 mg QD for 14 days. The highest exposure was determined at 500 mg QD with a geometric mean AUC_{τ} of 213 000 ng×h/mL and Cₘₐₓ of 15 100 ng/mL. Analysis of plasma obtained at Day 14 demonstrated the presence of 1 major metabolite, the p-hydroxylated Compound I.

Similar exposure was observed between the ALT1 suspension and ALT1 tablet and between the ALT1 tablet when given with or without food based on AUC geometric mean ratios being contained within the acceptance limits for bioequivalence (80% to 125%).

Meanwhile, Compound I has been found to possess half-life in human subjects to be about 13 to 15 hours, much longer than other RIPK1 inhibitors in the art (for example 2 to 3 hours for GSK2982772).

### Example 2

Compound I was study in multiple toxicology studies up to 6- and 9-months in duration have been conducted in rats and monkeys respectively. While minimal effects have been noted in rats, mortality related to secondary infections have been noted in monkeys at doses ≥250 mg/kg/day. The safety pharmacology and genotoxicity battery found no concerning effects for human safety. Additionally, the developmental and reproductive toxicology studies conducted in rats and rabbits did not reveal any concerns for women of childbearing potential. Surprisingly, the NOAELs/NOELs established from each study and corresponding margins of safety (Table 2) support a human dose of only about 125 mg,

TABLE 2
Margin of Safety for Oral Administration of
Compound I Based on Estimated Human Exposure

| | Dose | AUC₀₋₂₄ | Margin of Safety^{a} | |
|---|---|---|---|---|
| | (mg/kg/day) | (ng · hr/mL) | 50 mg | 125 mg |
| Human dose 50 mg^{b} | 0.7 | 16040 | | |
| Human dose 125 mg^{b} | 1.8 | 40100 | | |
| Rat NOAEL^{c} | 1000 | 1080000 | 67 X | 27 X |
| Monkey NOAEL^{d} | 150 | 278000 | 17 X | 7 X |
| Rat EFD NOEL^{e} | 1000 | 1120000 | 70 X | 28 X |
| Rabbit EFD NOEL^{f} | 120 | 464000 | 29 X | 12 X |

Abbreviations: AUC₀₋₂₄ = area under the plasma concentration-time curve during the steady-state dosing interval of 24 hr; NOAEL = no-observed-adverse-effect-level; NOEL = no-observed-effect-level.
^{a}Margin of safety is the calculated AUC in animals/(predicted) AUC in humans.
^{b}AUC estimated based on the AUC_{0-∞} at a single dose of 125 mg in phase 1 study Part 3.
^{c}NOAEL determined in 6-month repeat-dose toxicity study; plasma toxicokinetics determined on Day 182 (AUC₀₋₂₄ in male and female rats were not determined to be significantly different).
^{d}NOAEL determined in 9-month repeat-dose toxicity study; plasma toxicokinetics determined on Day 273 (AUC₀₋₂₄ in male and female monkeys was similar).
^{e}NOEL determined in rat embryofetal development study; plasma toxicokinetics determined on Gestational Day 17 (AUC₀₋₂₄).
^{f}NOEL determined in rabbit embryofetal development study; plasma toxicokinetics determined on Gestational Day 19 (AUC₀₋₂₄).

### Example 3

Compound I was further studied in a Phase 1, open-label, single-dose, fixed-sequence study to evaluate the PK, safety, and tolerability of a single 125-mg dose administered as a solid dispersion oral suspension and a crystalline freebase tablet in healthy participants. Each participant received both formulations in 2 separate dosing periods.

PK parameters for Compound I after oral administration of a crystalline freebase tablet and a solid dispersion oral suspension are presented in Table 3. Surprisingly, there was a statistically significant decrease of approximately 70% for AUC (0-tₗₐₛₜ) and AUC (0-0), and 77% for Cₘₐₓ for the tablet compared with the oral suspension. The median tₘₐₓ was longer for the tablet compared with the oral suspension. The geometric mean t_{1/2} was similar between formulations. The amount of drug excreted unchanged between time zero and 24 hours postdose, Ae (0-24), and fraction of dose excreted unchanged between time zero and 24 hours postdose, Fe (0-24), of Compound I was lower for tablet compared with the oral suspension due to greater plasma exposure for the oral suspension, but CLr was comparable between formulations. Between-participant CV for AUC (0-tₗₐₛₜ), AUC (0-∞), and Cₘₐₓ was higher for the tablet compared with the oral suspension. CV ranged from 33.3% to 34.0% for the tablet formulation and 21.3% to 23.2% for the oral suspension formulation. The AUC (0 ∞) and Cₘₐₓ of the tablet was approximately 70% and 77%, respectively, lower than the suspension when each formulation was given as a single oral dose of 125 mg.

TABLE 3
Summary of Pharmacokinetic Parameters

| | | 125 mg Compound | | 125 mg Compound | |
|---|---|---|---|---|---|
| | | I Oral Suspension | | I Tablet | |
| Matrix | Parameter | (N = 15) | | (N = 13) | |
| Plasma | AUC(0-24) (ng · hr/mL) | 35300 | (21.5) [15] | 9470 | (30.1) [13] |
| | AUC(0-tₗₐₛₜ) (ng · hr/mL) | 45900 | (23.2) [14] | 14100 | (33.3) [13] |
| | AUC(0-∞) (ng · hr/mL) | 47300 | (23.1) [15] | 14700 | (34.0) [13] |
| | % AUC(tₗₐₛₜ-∞) (%) | 1.89 | (125.9) [15] | 2.80 | (91.0) [13] |
| | Cₘₐₓ (ng/mL) | 3050 | (21.3) [15] | 701 | (33.9) [13] |
| | tₘₐₓ (hr) | 1.50 | (0.500-5.00) [15] | 4.00 | (3.00-8.00) [13] |
| | t_{1/2} (hr) | 12.2 | (9.62-16.7) [15] | 13.5 | (8.49-24.5) [13] |
| | CL/F (L/day) | 63.4 | (23.1) [15] | 204 | (34.0) [13] |
| | Vz/F (L) | 46.5 | (22.8) [15] | 166 | (41.7) [13] |
| | Vss/F (L) | 46.3 | (22.4) [15] | 193 | (30.9) [13] |
| Urine | Ae(0-24) (mg) | 12.2 | (28.2) [15] | 3.68 | (47.7) [12] |
| | Fe(0-24) (%) | 9.73 | (28.2) [15] | 2.94 | (47.7) [12] |
| | CLᵣ (L/hr) | 0.344 | (33.7) [15] | 0.388 | (50.1) [12] |

Abbreviations: % AUC(tₗₐₛₜ-∞) = percentage of AUC(0-∞) extrapolated; Ae(0-24) = amount of drug excreted unchanged between time zero and 24 hours postdose; AUC(0-24) = area under the concentration versus time curve from time zero to 24 hours postdose during 1 dosing interval (i.e., 24 hr); AUC(0-∞) = area under the concentration versus time curve from zero to infinity; AUC(0-tₗₐₛₜ) = area under the concentration versus time curve from time zero to time t, where t is the last time point with a measurable concentration; CL/F = apparent total body clearance of drug calculated after extra-vascular administration; CLᵣ = renal clearance; Cₘₐₓ = maximum observed drug concentration; CV = coefficient of variation (%); Fe(0-24) = fraction of dose excreted unchanged between time zero and 24 hours postdose; n = number of participants with valid observations; N = number of participants; t_{1/2} = apparent terminal elimination half-life; tₘₐₓ = time to maximum observed drug concentration; Vss/F = apparent volume of distribution at steady state after extra-vascular administration; Vz/F = apparent volume of distribution during the terminal phase after extra-vascular administration; Geometric mean (CV) [n] statistics presented; for tₘₐₓ, median (minimum-maximum) [n] statistics presented; for t_{1/2}, geometric mean (minimum-maximum) [n] presented.

### Example 4

### Abbreviated Protocol:

An Adaptive Phase 2a/2b, Randomized, Double-Blind, Placebo-Controlled Study of Compound I in Adult Participants with Moderately-to-Severely Active Rheumatoid Arthritis

### Rationale:

This adaptive design Phase 2a/2b study aims to evaluate the efficacy and safety of multiple dose levels of Compound I in adult participants with moderately-to-severely active rheumatoid arthritis (RA). This is the first study of Compound I in RA.

### Objectives, Endpoints, and Estimands:

| Objectives | Endpoints |
|---|---|
| Primary | |
| Phase 2a | |
| To evaluate the efficacy of Compound I | Change in DAS28-hsCRP from baseline |
| compared to placebo as measured by changes | to Week 12 |
| in DAS28-hsCRP | |
| Phase 2b | |
| To evaluate the efficacy of Compound I | Proportion of participants achieving |
| compared to placebo in achieving ACR50 | ACR50 at Week 12 |
| Secondary | |
| To evaluate the effect of Compound I | Phase 2a: Proportions of participants |
| compared to placebo for measures of disease | achieving ACR20/50/70 at Week 12 |
| activity | Phase 2b: |
| | Change in DAS28-hsCRP from |
| | baseline to Week 12 |
| | Proportions of participants achieving |
| | ACR20/70 at Week 12 |
| | Changes from baseline to Week 12 for |
| | mean |
| | SDAI |
| | CDAI |
| | Change from baseline to Week 12 ACR |
| | core set values |
| | 68 tender joint counts |
| | 66 swollen joint counts, and |
| | PhGADA_VAS |
| To evaluate the effect of Compound I | Change from baseline to Week 12 for |
| compared to placebo for patient-reported | patient-reported ACR core set values |
| outcome measures | PaGADA_VAS |
| | Patient's Assessment of Arthritis Pain |
| | (VAS), and |
| | patient's assessment of physical |
| | function using HAQ-DI |
| | Change from baseline to Week 12 for |
| | the duration and severity of morning joint stiffness |
| | FACIT-F scores |
| | Change from baseline at Week 12 for |
| | SF-36 domains |
| | SF-36 Physical Component |
| | Summary, and |
| | SF-36 Mental Component Summary |
| To assess clinical disease activity following | Proportion of participants achieving LDA |
| treatment with Compound I | or remission at Week 24 for |
| | SDAI, and |
| | CDAI |
| | Proportion of participants achieving |
| | ACR20/50/70 at Week 24 |
| | Change in DAS28-hsCRP from baseline |
| | to Week 24 |
| | Change from baseline at Week 24 for |
| | ACR core set values |
| | 68 tender joint count |
| | 66 swollen joint count, and |
| | PhGADA_VAS |
| | Change from baseline at Week 24 for |
| | mean |
| | SDAI, and |
| | CDAI |
| To characterize the pharmacokinetics of | Trough plasma concentrations of |
| Compound I | Compound I |

Abbreviations: ACR = American College of Rheumatology; CDAI = Clinical Disease Activity Index; DAS28-hsCRP = Disease Activity Score - high-sensitivity C-reactive protein; FACIT-F = Functional Assessment of Chronic Illness Therapy-Fatigue; HAQ-DI = Health Assessment Questionnaire - Disability Index; LDA = low disease activity; PaGADA = Patient's Global Assessment of Disease Activity; PhGADA = Physician's Global Assessment of Disease Activity; SDAI = Simplified Disease Activity Index; SF-36 = Short Form-36 version 2 health survey acute form; VAS = visual analog scale.

### Primary Estimands

### Phase 2a

The primary clinical question of interest is:
What is the difference between Compound I and placebo in the target patient population, in mean change from baseline at Week 12 if restrictions pertaining to changing permitted concomitant medications, and taking prohibited medications were not violated, and all participants adhered to the study intervention?

The estimand is described by the following attributes:
Population: Participants with moderately-to-severely active RA, defined by the presence of ≥6 swollen joints based on 66 joint count and >6 tender joints based on 68 joint count at screening (Visit 1) and randomization (Visit 2/baseline)
Endpoint: Change from baseline for Disease Activity Score-high-sensitivity C-reactive protein (DAS28-hsCRP)
Intercurrent events (ICEs) related to study intervention include the use of prohibited medication, the change in permitted concomitant medications, and early discontinuation from the study or study intervention. A hypothetical estimand strategy will be used; that is, data collected after the first occurrence of ICE will be excluded from an analysis and what the treatment effect would have been if prohibited medication was not used, permitted concomitant medications did not change, and all participants adhered to the treatment will be estimated.
Population-level summary: Difference in mean change from baseline to Week 12 between study intervention conditions.
Rationale for the estimand: The data collected after the treatment discontinuation, the initiation of prohibited medication, or the change in permitted concomitant medications will not present the true efficacy effects.

### Phase 2b

The primary clinical question of interest is:
What is the difference between Compound I and placebo in the target patient population, in achieving a successful response at Week 12 without changing permitted concomitant medications, taking prohibited medications, or discontinuing the study intervention?

The estimand is described by the following attributes:
Population: Participants with moderately-to-severely active RA, defined by the presence of ≥6 swollen joints based on 66 joint count and >6 tender joints based on 68 joint count at screening (Visit 1) and randomization (Visit 2/baseline).
Endpoint: American College of Rheumatology (ACR) 50 at Week 12.
ICEs related to study intervention include the use of prohibited medication, the change in permitted concomitant medications, and early discontinuation from the study or study intervention. A composite strategy will be used; that is, a participant with ICEs will be considered as a nonresponder because it is assumed the participant was not receiving benefit from the intervention.
Population-level summary: Difference in proportion of participants achieving response at Week 12 between study intervention conditions.
Rationale for the estimand: The data collected after the treatment discontinuation, the initiation of prohibited medication, or the change in permitted concomitant medications will be categorized as treatment failures because it is assumed the participant was not receiving benefit from the intervention.

### Overall Design:

This study is an adaptive Phase 2a/2b multicenter, randomized, double-blind study to evaluate the efficacy and safety of multiple dose levels of Compound I in adult participants with moderately-to-severely active RA. This table summarizes the key characteristics for the 2 phases of the study.

| Study characteristics | Phase 2a | Phase 2b |
|---|---|---|
| Type of study | Proof of concept | Dose ranging |

| | |
|---|---|
| Participant population | Participants randomly assigned to study intervention will have a |
| | history of failure^{a} to at least 1 csDMARD, and either 1 |
| | bDMARD or 1 tsDMARD treatment. |
| Standard-of-care (SOC) | At Week 14, the following participants may receive the SOC^{b} |
| therapy | therapy at the investigator's discretion |
| | all participants randomly assigned to placebo |
| | all participants randomly assigned to Compound I who do |
| | not achieve low disease activity (CDAI ≤10) at Week 14. |
| | These participants will no longer receive the study |
| | intervention. |

Abbreviations: bDMARD = biologic disease-modifying antirheumatic drug; CDAI = Clinical Disease Activity Index; csDMARD = conventional synthetic disease-modifying antirheumatic drug; SJC = swollen joint count; TJS = tender joint count; tsDMARD = targeted synthetic disease-modifying antirheumatic drug.
^{a}History of failure = an inadequate response, intolerance, or loss of response.
^{b}The SOC therapy includes all the csDMARDs and b/tsDMARDs.

### Study Periods

Both Phase 2a and Phase 2b will have the following study periods:
Period I: screening (Visit 1)
Period II: double-blind, placebo-controlled treatment
Period III: treatment
Period IV: post-treatment follow-up (Visits 801 and 802)

### Treatment Periods

This table summarizes the key features of the treatment periods for both phases of the study. Study intervention will be self-administered by participants at home except for the day of any study visit.

| Treatment period | Phase 2a | Phase 2b |
|---|---|---|
| Period II, double-blind, | At the baseline visit (Visit 2), | At the baseline visit (Visit 2), |
| placebo-controlled treatment | participants who met the | participants who met the |
| | eligibility criteria will be | eligibility criteria will be |
| | randomly assigned, in a ratio of | randomly assigned, in a ratio of |
| | 2:1, to the following study | 2:1:2:2, to the following |
| | interventions: | planned study interventions: |
| | 50 mg QD Compound I, | 125 mg QD Compound I |
| | or | 50 mg QD Compound I |
| | placebo. | 25 mg QD Compound I, |
| | | or |
| | | placebo. |

| | |
|---|---|
| Period III: treatment | At Week 14, the following participants may begin standard-of-care |
| | therapy at the investigator's discretion: |
| | all placebo participants, and |
| | Compound I participants who have not achieved low disease |
| | activity (CDAI ≤10). These participants will no longer |
| | receive the study intervention. |
| | Participants receiving Compound I who have achieved low disease |
| | activity (CDAI ≤10) will continue their assigned dose and dosing |
| | frequency. |

Abbreviations: CDAI = Clinical Disease Activity Index; QD = once daily.

### BRIEF SUMMARY

The purpose of this study is to evaluate the efficacy and safety of Compound I compared to placebo in adults with moderately-to-severely active RA.

For each of the 2 phases of the study
The study duration will be up to 32 weeks.
The treatment duration will be up to 24 weeks.

During the treatment period, the visit frequency will be
Phase 2a: every 2 weeks, and
Phase 2b: every 2 to 4 weeks.

### Study Population:

Individuals included in this study
will be at least 18 years old
have been diagnosed with RA as an adult, at least 3 months prior to screening
currently have moderately-to-severely active RA, and
have had a history of failure (an inadequate response, intolerance, or loss of response) to at least 1 csDMARD and either 1 bDMARD or 1 tsDMARD treatment.

### Number of Participants:

Approximately 100 participants will be randomly assigned to study intervention (placebo or 50 mg QD) in Phase 2a.

If a higher dose cohort is activated in Phase 2a, up to approximately 100 additional participants could randomly assigned to study intervention (placebo or higher dose not to exceed a maximum of 125 mg).

Approximately 280 participants will be randomly assigned to study intervention in Phase 2b.

### Intervention Groups and Duration:

These tables list the interventions used in different phases of this clinical study. For both Phase 2a and 2b of the study, the treatment duration will be
12 weeks for the double-blind, placebo-controlled treatment, and
24 weeks for the total treatment period (including the placebo-controlled treatment).

As indicated in the “Overall Design” section of this Synopsis, the following will be allowed for qualifying participants at investigator's discretion:
Phases 2a and 2b: standard-of-care (SOC) therapy at Week 14

Phase 2a

| Intervention Name | Compound I^{a} | Placebo |
|---|---|---|
| Dose Level | 50 mg | Not |
| | | applicable |
| Frequency of Administration | QD | QD |
| Route of Administration | Oral | Oral |
| Authorized as Defined by EU | Not | Not |
| Clinical Trial Regulation | authorized | authorized |

^{a}A higher dose level cohort, not to exceed a maximum of 125 mg, may be added during Phase 2a based on the results of the interim analyses and recommendations of the sponsor's internal assessment committee (IAC).

Phase 2b

| | | |
|---|---|---|
| Intervention Name | Compound I^{a} | Placebo |

| | | | |
|---|---|---|---|
| Planned Dose Levels | 125 | mg | Not |
| | 50 | mg | applicable |
| | 25 | mg | |

| | | |
|---|---|---|
| Frequency of Administration | QD | QD |
| Route of Administration | Oral | Oral |
| Authorized as Defined by EU | Not | Not |
| Clinical Trial Regulation | authorized | authorized |

^{a}The final doses of Compound I in Phase 2b will be determined based on the interim analyses from Phase 2a of this study and recommendations of the sponsor's IAC.

### Schedule of Activities (SoA)

Schedule of Activities for Phase 2a of this Study
Visit 1 procedures may be conducted over more than 1 day as long as all activities are completed within the allowable visit tolerance.
For early discontinuations that occur before the last visit in the treatment period, see the activities listed for ED in this table.

Schedule of Activities for Phase 2a of This Study

| | | | | | | | | | | | | | | | | Period IV | | |
|---|---|---|---|---|---|---|---|---|---|---|---|---|---|---|---|---|---|---|
| | | Period II | | | | | | | | | | | | | | Post- | | |
| | Period I | Double-Blind, Placebo- | | | | | | | Period III | | | | | | | Treatment | | |
| Visit | Screening | Controlled Treatment | | | | | | | Treatment | | | | | | | Follow-up | | |
| number | 1 | 2 | 3 | 4 | 5 | 6 | 7 | 8 | 9 | 10 | 11 | 12 | 13 | 14 | ED | 801 | 802 | Comments |
| Weeks from | — | — | 2 | 4 | 6 | 8 | 10 | 12 | 14 | 16 | 18 | 20 | 22 | 24 | — | 28 | 32 | V801 occurs |
| randomization | | | | | | | | | | | | | | | | | | 4 weeks |
| | | | | | | | | | | | | | | | | | | after V14 or |
| | | | | | | | | | | | | | | | | | | ED. |
| | | | | | | | | | | | | | | | | | | V802 occurs |
| | | | | | | | | | | | | | | | | | | 8 weeks |
| | | | | | | | | | | | | | | | | | | after V14 or |
| | | | | | | | | | | | | | | | | | | ED. |
| Visit | −42 to −1 | — | ±2 | ±2 | ±2 | ±2 | ±2 | ±2 | ±2 | ±2 | ±2 | ±2 | ±2 | ±2 | — | ±7 | ±7 | |
| interval | | | | | | | | | | | | | | | | | | |
| tolerance | | | | | | | | | | | | | | | | | | |
| (days) | | | | | | | | | | | | | | | | | | |
| Fasting visit | X | X | | | | | | X | | | | | | X | X | X | X | See “Lipid |
| | | | | | | | | | | | | | | | | | | panel” row |
| | | | | | | | | | | | | | | | | | | for details. |
| Informed | X | | | | | | | | | | | | | | | | | The ICF |
| consent | | | | | | | | | | | | | | | | | | must be |
| | | | | | | | | | | | | | | | | | | signed |
| | | | | | | | | | | | | | | | | | | before any |
| | | | | | | | | | | | | | | | | | | protocol- |
| | | | | | | | | | | | | | | | | | | specific |
| | | | | | | | | | | | | | | | | | | tests/ |
| | | | | | | | | | | | | | | | | | | procedures |
| | | | | | | | | | | | | | | | | | | are |
| | | | | | | | | | | | | | | | | | | performed. |
| | | | | | | | | | | | | | | | | | | See |
| | | | | | | | | | | | | | | | | | | Appendix |
| | | | | | | | | | | | | | | | | | | 10.1, |
| | | | | | | | | | | | | | | | | | | Section |
| | | | | | | | | | | | | | | | | | | 10.1.3, for |
| | | | | | | | | | | | | | | | | | | additional |
| | | | | | | | | | | | | | | | | | | details. |
| Inclusion | X | X | | | | | | | | | | | | | | | | Confirm |
| and | | | | | | | | | | | | | | | | | | inclusion/ |
| exclusion | | | | | | | | | | | | | | | | | | exclusion |
| criteria, | | | | | | | | | | | | | | | | | | criteria |
| review and | | | | | | | | | | | | | | | | | | before |
| confirm | | | | | | | | | | | | | | | | | | assignment |
| | | | | | | | | | | | | | | | | | | and |
| | | | | | | | | | | | | | | | | | | administration |
| | | | | | | | | | | | | | | | | | | of first |
| | | | | | | | | | | | | | | | | | | dose of |
| | | | | | | | | | | | | | | | | | | study |
| | | | | | | | | | | | | | | | | | | intervention. |
| Demographics | X | | | | | | | | | | | | | | | | | Includes |
| | | | | | | | | | | | | | | | | | | ethnicity |
| | | | | | | | | | | | | | | | | | | (United |
| | | | | | | | | | | | | | | | | | | States only), |
| | | | | | | | | | | | | | | | | | | year of birth, |
| | | | | | | | | | | | | | | | | | | sex, and race |
| | | | | | | | | | | | | | | | | | | (as allowed |
| | | | | | | | | | | | | | | | | | | per local |
| | | | | | | | | | | | | | | | | | | regulations). |
| Preexisting | X | | | | | | | | | | | | | | | | | Collect all |
| conditions | | | | | | | | | | | | | | | | | | ongoing |
| and medical | | | | | | | | | | | | | | | | | | conditions |
| history, | | | | | | | | | | | | | | | | | | and relevant |
| including | | | | | | | | | | | | | | | | | | past surgical |
| relevant | | | | | | | | | | | | | | | | | | and medical |
| surgical | | | | | | | | | | | | | | | | | | history. |
| history | | | | | | | | | | | | | | | | | | |
| Prespecified | X | | | | | | | | | | | | | | | | | Includes RA |
| medical | | | | | | | | | | | | | | | | | | diagnosis |
| history | | | | | | | | | | | | | | | | | | and onset |
| (indication | | | | | | | | | | | | | | | | | | and prior |
| and history | | | | | | | | | | | | | | | | | | malignancy. |
| of interest) | | | | | | | | | | | | | | | | | | |
| Prior | X | | | | | | | | | | | | | | | | | Any |
| treatments | | | | | | | | | | | | | | | | | | previous |
| for | | | | | | | | | | | | | | | | | | therapy for |
| indication | | | | | | | | | | | | | | | | | | RA in the |
| | | | | | | | | | | | | | | | | | | past 15 years |
| | | | | | | | | | | | | | | | | | | that has now |
| | | | | | | | | | | | | | | | | | | been |
| | | | | | | | | | | | | | | | | | | discontinued. |
| Substance | X | | | | | | | | | | | | | | | | | |
| use (alcohol, | | | | | | | | | | | | | | | | | | |
| caffeine, | | | | | | | | | | | | | | | | | | |
| tobacco use) | | | | | | | | | | | | | | | | | | |
| Concomitant | X | X | X | X | X | X | X | X | X | X | X | X | X | X | X | X | X | Collect |
| medications | | | | | | | | | | | | | | | | | | additional |
| | | | | | | | | | | | | | | | | | | data for |
| | | | | | | | | | | | | | | | | | | concomitant |
| | | | | | | | | | | | | | | | | | | medications |
| | | | | | | | | | | | | | | | | | | of interest |
| | | | | | | | | | | | | | | | | | | (that is, |
| | | | | | | | | | | | | | | | | | | permitted |
| | | | | | | | | | | | | | | | | | | therapies for |
| | | | | | | | | | | | | | | | | | | RA; |
| | | | | | | | | | | | | | | | | | | Sections 6.9.1 |
| | | | | | | | | | | | | | | | | | | and 6.9.2). |
| Adverse | X | X | X | X | X | X | X | X | X | X | X | X | X | X | X | X | X | Any events |
| events (AEs) | | | | | | | | | | | | | | | | | | that occur |
| | | | | | | | | | | | | | | | | | | after signing |
| | | | | | | | | | | | | | | | | | | the ICF are |
| | | | | | | | | | | | | | | | | | | considered |
| | | | | | | | | | | | | | | | | | | AEs as |
| | | | | | | | | | | | | | | | | | | defined in |
| | | | | | | | | | | | | | | | | | | Section 8.3.1. |
| | | | | | | | | | | | | | | | | | | For |
| | | | | | | | | | | | | | | | | | | AESIs, |
| | | | | | | | | | | | | | | | | | | collect |
| | | | | | | | | | | | | | | | | | | additional |
| | | | | | | | | | | | | | | | | | | data |
| | | | | | | | | | | | | | | | | | | (Section |
| | | | | | | | | | | | | | | | | | | 8.3.3). |

Physical evaluation

| Height | X | | | | | | | | | | | | | | | | | Participant |
|---|---|---|---|---|---|---|---|---|---|---|---|---|---|---|---|---|---|---|
| | | | | | | | | | | | | | | | | | | should |
| | | | | | | | | | | | | | | | | | | remove |
| | | | | | | | | | | | | | | | | | | shoes. |
| Weight | X | X | | | | | | X | | | | | | | X | X | X | |
| Vital signs | X | X | X | X | X | X | X | X | X | X | X | X | X | X | X | X | X | Includes |
| | | | | | | | | | | | | | | | | | | blood |
| | | | | | | | | | | | | | | | | | | pressure, |
| | | | | | | | | | | | | | | | | | | body |
| | | | | | | | | | | | | | | | | | | temperature, |
| | | | | | | | | | | | | | | | | | | and pulse |
| | | | | | | | | | | | | | | | | | | rate. |
| | | | | | | | | | | | | | | | | | | Measure |
| | | | | | | | | | | | | | | | | | | vital signs |
| | | | | | | | | | | | | | | | | | | after |
| | | | | | | | | | | | | | | | | | | participant |
| | | | | | | | | | | | | | | | | | | has been |
| | | | | | | | | | | | | | | | | | | sitting at |
| | | | | | | | | | | | | | | | | | | least |
| | | | | | | | | | | | | | | | | | | 5 minutes, |
| | | | | | | | | | | | | | | | | | | before |
| | | | | | | | | | | | | | | | | | | obtaining an |
| | | | | | | | | | | | | | | | | | | ECG |
| | | | | | | | | | | | | | | | | | | tracing, and |
| | | | | | | | | | | | | | | | | | | before |
| | | | | | | | | | | | | | | | | | | collection of |
| | | | | | | | | | | | | | | | | | | blood |
| | | | | | | | | | | | | | | | | | | samples for |
| | | | | | | | | | | | | | | | | | | laboratory |
| | | | | | | | | | | | | | | | | | | testing. |
| Physical | X | | | | | | | X | | | | | | | X | | X | See Section |
| examination | | | | | | | | | | | | | | | | | | 8.2.1. |
| | | | | | | | | | | | | | | | | | | The |
| | | | | | | | | | | | | | | | | | | complete |
| | | | | | | | | | | | | | | | | | | physical |
| | | | | | | | | | | | | | | | | | | examination |
| | | | | | | | | | | | | | | | | | | excludes |
| | | | | | | | | | | | | | | | | | | pelvic, |
| | | | | | | | | | | | | | | | | | | rectal, and |
| | | | | | | | | | | | | | | | | | | breast |
| | | | | | | | | | | | | | | | | | | examinations, |
| | | | | | | | | | | | | | | | | | | unless |
| | | | | | | | | | | | | | | | | | | clinically |
| | | | | | | | | | | | | | | | | | | indicated. |
| Symptom- | | X | | | | | | | | | | | | | X | X | | See Section |
| directed | | | | | | | | | | | | | | | | | | 8.2.1. |
| physical | | | | | | | | | | | | | | | | | | Conduct at |
| assessment | | | | | | | | | | | | | | | | | | the |
| | | | | | | | | | | | | | | | | | | discretion of |
| | | | | | | | | | | | | | | | | | | the |
| | | | | | | | | | | | | | | | | | | investigator |
| | | | | | | | | | | | | | | | | | | or qualified |
| | | | | | | | | | | | | | | | | | | personnel |
| | | | | | | | | | | | | | | | | | | per local |
| | | | | | | | | | | | | | | | | | | regulations, |
| | | | | | | | | | | | | | | | | | | as indicated |
| | | | | | | | | | | | | | | | | | | based on |
| | | | | | | | | | | | | | | | | | | participant |
| | | | | | | | | | | | | | | | | | | status and |
| | | | | | | | | | | | | | | | | | | standard of |
| | | | | | | | | | | | | | | | | | | care. |
| Tuberculosis | | | | | | | | X | | | | | | | X | | X | See Sections |
| (TB) | | | | | | | | | | | | | | | | | | 8.2.1 and |
| monitoring | | | | | | | | | | | | | | | | | | 8.2.9. |
| and thyroid | | | | | | | | | | | | | | | | | | |
| palpation | | | | | | | | | | | | | | | | | | |
| 12-lead | X | X | X | X | | X | | X | X | X | | X | | X | X | X | X | ECGs |
| ECG | | | | | | | | | | | | | | | | | | should be |
| (centrally | | | | | | | | | | | | | | | | | | recorded |
| read and | | | | | | | | | | | | | | | | | | prior to |
| locally | | | | | | | | | | | | | | | | | | collection of |
| interpreted) | | | | | | | | | | | | | | | | | | blood |
| | | | | | | | | | | | | | | | | | | samples for |
| | | | | | | | | | | | | | | | | | | any |
| | | | | | | | | | | | | | | | | | | laboratory |
| | | | | | | | | | | | | | | | | | | testing. |
| | | | | | | | | | | | | | | | | | | Local ECGs |
| | | | | | | | | | | | | | | | | | | may be |
| | | | | | | | | | | | | | | | | | | obtained at |
| | | | | | | | | | | | | | | | | | | additional |
| | | | | | | | | | | | | | | | | | | times, when |
| | | | | | | | | | | | | | | | | | | deemed |
| | | | | | | | | | | | | | | | | | | clinically |
| | | | | | | | | | | | | | | | | | | necessary. |
| | | | | | | | | | | | | | | | | | | See Section |
| | | | | | | | | | | | | | | | | | | 8.2.3. |
| Chest x-ray | X | | | | | | | | | | | | | | | | | See Section |
| (posterior- | | | | | | | | | | | | | | | | | | 8.2.4. |
| anterior and | | | | | | | | | | | | | | | | | | Not done at |
| lateral view; | | | | | | | | | | | | | | | | | | screening if |
| local) | | | | | | | | | | | | | | | | | | done |
| | | | | | | | | | | | | | | | | | | within |
| | | | | | | | | | | | | | | | | | | 3 months |
| | | | | | | | | | | | | | | | | | | before |
| | | | | | | | | | | | | | | | | | | screening, |
| | | | | | | | | | | | | | | | | | | and |
| | | | | | | | | | | | | | | | | | | documentation |
| | | | | | | | | | | | | | | | | | | of the |
| | | | | | | | | | | | | | | | | | | previous |
| | | | | | | | | | | | | | | | | | | chest X- |
| | | | | | | | | | | | | | | | | | | ray is |
| | | | | | | | | | | | | | | | | | | available. |
| | | | | | | | | | | | | | | | | | | Interpreted |
| | | | | | | | | | | | | | | | | | | and reported |
| | | | | | | | | | | | | | | | | | | by |
| | | | | | | | | | | | | | | | | | | radiologist |
| | | | | | | | | | | | | | | | | | | or |
| | | | | | | | | | | | | | | | | | | pulmonologist. |
| Schedule | X | | | | | X | | | | | | | | | | | | If not |
| MRI | | | | | | | | | | | | | | | | | | completed at |
| | | | | | | | | | | | | | | | | | | V6, this will |
| | | | | | | | | | | | | | | | | | | not be |
| | | | | | | | | | | | | | | | | | | considered a |
| | | | | | | | | | | | | | | | | | | protocol |
| | | | | | | | | | | | | | | | | | | deviation. |
| MRI | X | | | | | | | X | | | | | | | | | | At |
| (hand/wrist) | | | | | | | | | | | | | | | | | | screening, |
| | | | | | | | | | | | | | | | | | | perform |
| | | | | | | | | | | | | | | | | | | MRI only |
| | | | | | | | | | | | | | | | | | | after all |
| | | | | | | | | | | | | | | | | | | eligibility |
| | | | | | | | | | | | | | | | | | | criteria are |
| | | | | | | | | | | | | | | | | | | met except |
| | | | | | | | | | | | | | | | | | | Inclusion |
| | | | | | | | | | | | | | | | | | | Criterion [8] |
| | | | | | | | | | | | | | | | | | | (exception |
| | | | | | | | | | | | | | | | | | | can be |
| | | | | | | | | | | | | | | | | | | considered |
| | | | | | | | | | | | | | | | | | | after |
| | | | | | | | | | | | | | | | | | | consultation |
| | | | | | | | | | | | | | | | | | | with the |
| | | | | | | | | | | | | | | | | | | sponsor). |
| | | | | | | | | | | | | | | | | | | Evaluate the |
| | | | | | | | | | | | | | | | | | | same |
| | | | | | | | | | | | | | | | | | | hand/wrist at |
| | | | | | | | | | | | | | | | | | | screening |
| | | | | | | | | | | | | | | | | | | and at Week |
| | | | | | | | | | | | | | | | | | | 12. |
| | | | | | | | | | | | | | | | | | | At Week 12, |
| | | | | | | | | | | | | | | | | | | MRI may be |
| | | | | | | | | | | | | | | | | | | completed |
| | | | | | | | | | | | | | | | | | | ±7 days of |
| | | | | | | | | | | | | | | | | | | the planned |
| | | | | | | | | | | | | | | | | | | Week 12 |
| | | | | | | | | | | | | | | | | | | visit. |
| Patient-Reported Outcomes (Electronic) | | | | | | | | | | | | | | | | | | Complete |
| | | | | | | | | | | | | | | | | | | before any |
| | | | | | | | | | | | | | | | | | | clinician- |
| | | | | | | | | | | | | | | | | | | administered |
| | | | | | | | | | | | | | | | | | | assessments |
| | | | | | | | | | | | | | | | | | | and dosing, |
| | | | | | | | | | | | | | | | | | | in the order |
| | | | | | | | | | | | | | | | | | | listed here. |
| Patient's | X | X | X | X | X | X | X | X | X | X | X | X | X | X | X | X | X | |
| Global | | | | | | | | | | | | | | | | | | |
| Assessment | | | | | | | | | | | | | | | | | | |
| of Arthritis | | | | | | | | | | | | | | | | | | |
| Pain (VAS) | | | | | | | | | | | | | | | | | | |
| Health | X | X | X | X | X | X | X | X | X | X | X | X | X | X | X | X | X | |
| Assessment | | | | | | | | | | | | | | | | | | |
| Questionnaire- | | | | | | | | | | | | | | | | | | |
| Disability | | | | | | | | | | | | | | | | | | |
| Index | | | | | | | | | | | | | | | | | | |
| (HAQ-DI) | | | | | | | | | | | | | | | | | | |
| Patient's | X | X | X | X | X | X | X | X | X | X | X | X | X | X | X | X | X | |
| Global | | | | | | | | | | | | | | | | | | |
| Assessment | | | | | | | | | | | | | | | | | | |
| of Disease | | | | | | | | | | | | | | | | | | |
| Activity | | | | | | | | | | | | | | | | | | |
| (RA) | | | | | | | | | | | | | | | | | | |
| (PaGADA_ | | | | | | | | | | | | | | | | | | |
| VAS) | | | | | | | | | | | | | | | | | | |
| Medical | | X | | | | | | X | | | | | | X | X | | X | |
| Outcomes | | | | | | | | | | | | | | | | | | |
| Study 36- | | | | | | | | | | | | | | | | | | |
| Item Short | | | | | | | | | | | | | | | | | | |
| Form Health | | | | | | | | | | | | | | | | | | |
| Survey (SF- | | | | | | | | | | | | | | | | | | |
| 36 | | | | | | | | | | | | | | | | | | |
| v2.Acute) | | | | | | | | | | | | | | | | | | |
| Morning | | X | X | X | X | X | X | X | X | X | X | X | X | X | X | X | X | |
| Joint | | | | | | | | | | | | | | | | | | |
| Stiffness | | | | | | | | | | | | | | | | | | |
| Duration | | | | | | | | | | | | | | | | | | |
| PRO (24-hr | | | | | | | | | | | | | | | | | | |
| recall) | | | | | | | | | | | | | | | | | | |
| Morning | | X | X | X | X | X | X | X | X | X | X | X | X | X | X | X | X | |
| Joint | | | | | | | | | | | | | | | | | | |
| Stiffness | | | | | | | | | | | | | | | | | | |
| Severity | | | | | | | | | | | | | | | | | | |
| (NRS) | | | | | | | | | | | | | | | | | | |
| (day of visit) | | | | | | | | | | | | | | | | | | |
| FACIT-F | | X | X | X | X | X | X | X | X | X | X | X | X | X | X | X | X | |
| Clinician-Administered Assessments | | | | | | | | | | | | | | | | | | Determine |
| (Electronic) | | | | | | | | | | | | | | | | | | before |
| | | | | | | | | | | | | | | | | | | dosing. |
| Clinician- | X | X | X | X | X | X | X | X | X | X | X | X | X | X | X | X | X | The Joint |
| Administered | | | | | | | | | | | | | | | | | | Assessor |
| Tender/Swollen | | | | | | | | | | | | | | | | | | will perform |
| Joint | | | | | | | | | | | | | | | | | | the joint |
| Count | | | | | | | | | | | | | | | | | | counts. See |
| (TSJC) | | | | | | | | | | | | | | | | | | Section 8. |
| (68/66) | | | | | | | | | | | | | | | | | | |
| Physician | X | X | X | X | X | X | X | X | X | X | X | X | X | X | X | X | X | |
| Global | | | | | | | | | | | | | | | | | | |
| Assessment | | | | | | | | | | | | | | | | | | |
| of Disease | | | | | | | | | | | | | | | | | | |
| Activity | | | | | | | | | | | | | | | | | | |
| (PhGADA) | | | | | | | | | | | | | | | | | | |

Clinician-Administered Assessments
(Paper)

| C-SSRS | X | | | | | | | | | | | | | | | | | Collect AEs |
|---|---|---|---|---|---|---|---|---|---|---|---|---|---|---|---|---|---|---|
| Screening/ | | | | | | | | | | | | | | | | | | before the |
| Baseline | | | | | | | | | | | | | | | | | | collection of |
| C-SSRS | | X | X | X | X | X | X | X | X | X | X | X | X | X | X | X | X | the C-SSRS. |
| Since Last | | | | | | | | | | | | | | | | | | |
| Assessed | | | | | | | | | | | | | | | | | | |
| Actigraphy | | | | | | | | | | | | | | | | | | |
| Actigraphy | X | | | | | | X | | | | | | X | | | | | See |
| device | | | | | | | | | | | | | | | | | | Appendix |
| dispense | | | | | | | | | | | | | | | | | | 10.9. |
| Actigraphy | | X | | | | | | X | | | | | | X | X | | | Participants |
| device | | | | | | | | | | | | | | | | | | not eligible |
| return | | | | | | | | | | | | | | | | | | for the study |
| | | | | | | | | | | | | | | | | | | should |
| | | | | | | | | | | | | | | | | | | return the |
| | | | | | | | | | | | | | | | | | | device upon |
| | | | | | | | | | | | | | | | | | | screen |
| | | | | | | | | | | | | | | | | | | failure. A |
| | | | | | | | | | | | | | | | | | | minimum of |
| | | | | | | | | | | | | | | | | | | 14 |
| | | | | | | | | | | | | | | | | | | continuous |
| | | | | | | | | | | | | | | | | | | days of |
| | | | | | | | | | | | | | | | | | | actigraphy |
| | | | | | | | | | | | | | | | | | | data should |
| | | | | | | | | | | | | | | | | | | be collected |
| | | | | | | | | | | | | | | | | | | before the |
| | | | | | | | | | | | | | | | | | | device is |
| | | | | | | | | | | | | | | | | | | returned. |
| | | | | | | | | | | | | | | | | | | However, |
| | | | | | | | | | | | | | | | | | | collection of |
| | | | | | | | | | | | | | | | | | | less than 14 |
| | | | | | | | | | | | | | | | | | | continuous |
| | | | | | | | | | | | | | | | | | | days of data |
| | | | | | | | | | | | | | | | | | | will not be |
| | | | | | | | | | | | | | | | | | | considered a |
| | | | | | | | | | | | | | | | | | | protocol |
| | | | | | | | | | | | | | | | | | | deviation. |
| | | | | | | | | | | | | | | | | | | See |
| | | | | | | | | | | | | | | | | | | Appendix |
| | | | | | | | | | | | | | | | | | | 10.9. |
| Laboratory Tests and Sample | | | | | | | | | | | | | | | | | | See |
| Collections | | | | | | | | | | | | | | | | | | Appendix |
| | | | | | | | | | | | | | | | | | | 10.2 for |
| | | | | | | | | | | | | | | | | | | details of |
| | | | | | | | | | | | | | | | | | | testing |
| | | | | | | | | | | | | | | | | | | included in |
| | | | | | | | | | | | | | | | | | | panels. |
| Hematology | X | X | X | X | X | X | X | X | X | X | X | X | X | X | X | X | X | Collect pre- |
| | | | | | | | | | | | | | | | | | | dose. |
| Erythrocyte | X | X | X | X | X | X | X | X | X | X | X | X | X | X | X | X | X | Collect pre- |
| sedimentation | | | | | | | | | | | | | | | | | | dose. |
| rate (ESR) | | | | | | | | | | | | | | | | | | |
| (local) | | | | | | | | | | | | | | | | | | |
| Clinical | X | X | X | X | X | X | X | X | X | X | X | X | X | X | X | X | X | Collect pre- |
| chemistry | | | | | | | | | | | | | | | | | | dose. |
| Lipid panel | X | X | | | | | | X | | | | | | X | X | X | X | Collect pre- |
| | | | | | | | | | | | | | | | | | | dose. |
| | | | | | | | | | | | | | | | | | | Participants |
| | | | | | | | | | | | | | | | | | | should not |
| | | | | | | | | | | | | | | | | | | eat or drink |
| | | | | | | | | | | | | | | | | | | anything but |
| | | | | | | | | | | | | | | | | | | water for 9- |
| | | | | | | | | | | | | | | | | | | 12 hours |
| | | | | | | | | | | | | | | | | | | before the |
| | | | | | | | | | | | | | | | | | | visit. Fasting |
| | | | | | | | | | | | | | | | | | | is not |
| | | | | | | | | | | | | | | | | | | required at |
| | | | | | | | | | | | | | | | | | | an ED visit. |
| | | | | | | | | | | | | | | | | | | If a |
| | | | | | | | | | | | | | | | | | | participant |
| | | | | | | | | | | | | | | | | | | attends these |
| | | | | | | | | | | | | | | | | | | visits in a |
| | | | | | | | | | | | | | | | | | | nonfasting |
| | | | | | | | | | | | | | | | | | | state, the |
| | | | | | | | | | | | | | | | | | | sample |
| | | | | | | | | | | | | | | | | | | should still |
| | | | | | | | | | | | | | | | | | | be collected. |
| | | | | | | | | | | | | | | | | | | This will not |
| | | | | | | | | | | | | | | | | | | be |
| | | | | | | | | | | | | | | | | | | considered a |
| | | | | | | | | | | | | | | | | | | protocol |
| | | | | | | | | | | | | | | | | | | deviation. |
| Thyroid | | X | | | | | | X | | | | | | X | X | X | X | Collect pre- |
| panel | | | | | | | | | | | | | | | | | | dose. |
| Coagulation | | X | | X | | X | | X | X | X | | X | | X | X | X | X | Collect pre- |
| | | | | | | | | | | | | | | | | | | dose. |
| ACTH | | X | | | | | | X | | | | | | X | | | X | Collect |
| | | | | | | | | | | | | | | | | | | between 8 |
| | | | | | | | | | | | | | | | | | | am and 10 |
| | | | | | | | | | | | | | | | | | | am. |
| Cortisol | | X | | | | | | X | | | | | | X | | | X | Collect pre- |
| | | | | | | | | | | | | | | | | | | dose. |
| Renin | | X | | | | | | X | | | | | | X | | | X | Collect pre- |
| DHEA-S | | X | | | | | | X | | | | | | X | | | X | dose. |
| Testosterone | | X | | | | | | X | | | | | | X | | | X | For women |
| | | | | | | | | | | | | | | | | | | only. Collect |
| | | | | | | | | | | | | | | | | | | between 8 |
| | | | | | | | | | | | | | | | | | | am and 10 |
| | | | | | | | | | | | | | | | | | | am. |
| | | | | | | | | | | | | | | | | | | Collect pre- |
| | | | | | | | | | | | | | | | | | | dose. |
| Urinalysis | X | X | | X | | | | X | | X | | | | X | X | X | | Collect pre- |
| | | | | | | | | | | | | | | | | | | dose. |
| Serum | X | | | | | | | | | | | | | | | | | Only for |
| pregnancy | | | | | | | | | | | | | | | | | | WOCBP. |
| | | | | | | | | | | | | | | | | | | See |
| | | | | | | | | | | | | | | | | | | Appendix |
| | | | | | | | | | | | | | | | | | | 10.4, |
| | | | | | | | | | | | | | | | | | | Sections |
| | | | | | | | | | | | | | | | | | | 10.4.1 and |
| | | | | | | | | | | | | | | | | | | 10.4.2. |
| Urine | | X | | X | | X | | X | | X | | X | | X | X | X | | Only for |
| pregnancy | | | | | | | | | | | | | | | | | | WOCBP. |
| (local) | | | | | | | | | | | | | | | | | | See Section |
| | | | | | | | | | | | | | | | | | | 8.2.6 and |
| | | | | | | | | | | | | | | | | | | Appendix |
| | | | | | | | | | | | | | | | | | | 10.4. |
| Follicle- | X | | | | | | | | | | | | | | | | | Optional; |
| stimulating | | | | | | | | | | | | | | | | | | perform |
| hormone | | | | | | | | | | | | | | | | | | only to |
| (FSH) | | | | | | | | | | | | | | | | | | confirm |
| | | | | | | | | | | | | | | | | | | postmenopausal |
| | | | | | | | | | | | | | | | | | | status. |
| C-reactive | X | X | X | X | X | X | X | X | X | X | X | X | X | X | X | X | X | Collect pre- |
| protein, | | | | | | | | | | | | | | | | | | dose. |
| high- | | | | | | | | | | | | | | | | | | |
| sensitivity | | | | | | | | | | | | | | | | | | |
| (hsCRP) | | | | | | | | | | | | | | | | | | |
| Rheumatoid | X | X | | | | | | X | | | | | | X | X | | X | Collect pre- |
| factor (RF) | | | | | | | | | | | | | | | | | | dose. |
| Anticyclic | X | X | | | | | | X | | | | | | X | X | | X | Collect pre- |
| citrullinated | | | | | | | | | | | | | | | | | | dose. |
| peptide | | | | | | | | | | | | | | | | | | |
| (anti-CCP) | | | | | | | | | | | | | | | | | | |
| Flow | | X | | | | | | X | | | | | | X | X | | X | Collect |
| cytometry | | | | | | | | | | | | | | | | | | samples pre- |
| panel | | | | | | | | | | | | | | | | | | dose. |
| Calprotectin | | X | X | X | | X | | X | | | | | | | X | | | |
| Immunoglobulin | | X | | | | | | X | | | | | | X | X | | X | |
| panel | | | | | | | | | | | | | | | | | | |
| Protein | | X | | X | | X | | X | | | | | | X | X | | X | |
| disease | | | | | | | | | | | | | | | | | | |
| activity | | | | | | | | | | | | | | | | | | |
| (DA) panel | | | | | | | | | | | | | | | | | | |
| Troponin | | X | | | | | | X | X | | | | | X | X | | X | |
| TB test | X | | | | | | | | | | | | | | | | | See |
| | | | | | | | | | | | | | | | | | | Appendix |
| | | | | | | | | | | | | | | | | | | 10.2. |
| | | | | | | | | | | | | | | | | | | Participants |
| | | | | | | | | | | | | | | | | | | who had a |
| | | | | | | | | | | | | | | | | | | tuberculin |
| | | | | | | | | | | | | | | | | | | skin test |
| | | | | | | | | | | | | | | | | | | (TST) must |
| | | | | | | | | | | | | | | | | | | have the test |
| | | | | | | | | | | | | | | | | | | read 48 to |
| | | | | | | | | | | | | | | | | | | 72 hours |
| | | | | | | | | | | | | | | | | | | after |
| | | | | | | | | | | | | | | | | | | placement. |
| | | | | | | | | | | | | | | | | | | The TST test |
| | | | | | | | | | | | | | | | | | | does not |
| | | | | | | | | | | | | | | | | | | need to be |
| | | | | | | | | | | | | | | | | | | read at the |
| | | | | | | | | | | | | | | | | | | site but must |
| | | | | | | | | | | | | | | | | | | be read by a |
| | | | | | | | | | | | | | | | | | | trained |
| | | | | | | | | | | | | | | | | | | professional |
| | | | | | | | | | | | | | | | | | | and results |
| | | | | | | | | | | | | | | | | | | must be |
| | | | | | | | | | | | | | | | | | | presented to |
| | | | | | | | | | | | | | | | | | | the site prior |
| | | | | | | | | | | | | | | | | | | to first dose |
| | | | | | | | | | | | | | | | | | | of study |
| | | | | | | | | | | | | | | | | | | intervention. |
| | | | | | | | | | | | | | | | | | | See Section |
| | | | | | | | | | | | | | | | | | | 8.2.9 and |
| | | | | | | | | | | | | | | | | | | Appendix |
| | | | | | | | | | | | | | | | | | | 10.7 |
| HIV | X | | | | | | | | | | | | | | | | | |
| screening | | | | | | | | | | | | | | | | | | |
| tests | | | | | | | | | | | | | | | | | | |
| Hepatitis C | X | | | | | | | | | | | | | | | | | If HCV |
| virus (HCV) | | | | | | | | | | | | | | | | | | antibody test |
| screening | | | | | | | | | | | | | | | | | | is positive, it |
| tests | | | | | | | | | | | | | | | | | | must be |
| | | | | | | | | | | | | | | | | | | followed by |
| | | | | | | | | | | | | | | | | | | an HCV |
| | | | | | | | | | | | | | | | | | | RNA test. |
| | | | | | | | | | | | | | | | | | | See Section |
| | | | | | | | | | | | | | | | | | | 8.2.11. |
| Hepatitis B | X | | | | | | | | | | | | | | | | | See |
| virus (HBV) | | | | | | | | | | | | | | | | | | Section 8.2. |
| screening | | | | | | | | | | | | | | | | | | 10 and |
| tests | | | | | | | | | | | | | | | | | | Appendix |
| | | | | | | | | | | | | | | | | | | 10.2. |
| Hepatitis B | X | | | | | | | X | | | | | | X | X | | X | Only for |
| virus (HBV) | | | | | | | | | | | | | | | | | | participants |
| DNA | | | | | | | | | | | | | | | | | | who are |
| | | | | | | | | | | | | | | | | | | positive for |
| | | | | | | | | | | | | | | | | | | anti-HBc at |
| | | | | | | | | | | | | | | | | | | screening. |
| | | | | | | | | | | | | | | | | | | See Section |
| | | | | | | | | | | | | | | | | | | 8.2.10. |
| Estimated | X | | | | | | | | | | | | | | | | | Calculated |
| glomerular | | | | | | | | | | | | | | | | | | using the |
| filtration | | | | | | | | | | | | | | | | | | CKD-EPI |
| rate (eGFR) | | | | | | | | | | | | | | | | | | creatinine |
| | | | | | | | | | | | | | | | | | | equation |
| | | | | | | | | | | | | | | | | | | (2021). |
| Pharmacokinetic | | X | X | X | | X | | X | | X | | X | | | | | | Collect PK |
| (PK) | | | | | | | | | | | | | | | | | | samples at |
| samples | | | | | | | | | | | | | | | | | | these times: |
| | | | | | | | | | | | | | | | | | | Visit 2: 1 to |
| | | | | | | | | | | | | | | | | | | 3 hours post- |
| | | | | | | | | | | | | | | | | | | dose. |
| | | | | | | | | | | | | | | | | | | Visits 3, 4: |
| | | | | | | | | | | | | | | | | | | pre-dose and |
| | | | | | | | | | | | | | | | | | | 1 to 3 hours |
| | | | | | | | | | | | | | | | | | | post-dose. |
| | | | | | | | | | | | | | | | | | | Visits 6, 8, |
| | | | | | | | | | | | | | | | | | | 10, 12: pre- |
| | | | | | | | | | | | | | | | | | | dose. |
| | | | | | | | | | | | | | | | | | | Note: |
| | | | | | | | | | | | | | | | | | | Collect PK |
| | | | | | | | | | | | | | | | | | | samples at |
| | | | | | | | | | | | | | | | | | | V10 and 12 |
| | | | | | | | | | | | | | | | | | | only in |
| | | | | | | | | | | | | | | | | | | participants |
| | | | | | | | | | | | | | | | | | | receiving |
| | | | | | | | | | | | | | | | | | | Compound |
| | | | | | | | | | | | | | | | | | | I. |

Stored
Samples

| | | | | | | | | |
|---|---|---|---|---|---|---|---|---|
| Genetics | X | | | | | | | Sample can |
| sample | | | | | | | | be obtained |
| | | | | | | | | at or after |
| | | | | | | | | the specified |
| | | | | | | | | visit. |
| Exploratory | X | X | X | X | X | X | X | Collect |
| biomarker | | | | | | | | samples pre- |
| samples | | | | | | | | dose. |

Randomization and
Dosing

| | | | | | | | | | | | | | | | | | | |
|---|---|---|---|---|---|---|---|---|---|---|---|---|---|---|---|---|---|---|
| Register | X | X | X | X | X | X | X | X | X | X | X | X | X | X | X | X | X | In the event |
| visit with | | | | | | | | | | | | | | | | | | of an |
| IWRS | | | | | | | | | | | | | | | | | | emergency |
| | | | | | | | | | | | | | | | | | | unblinding, |
| | | | | | | | | | | | | | | | | | | do not |
| | | | | | | | | | | | | | | | | | | register the |
| | | | | | | | | | | | | | | | | | | visit in |
| | | | | | | | | | | | | | | | | | | IWRS. |
| Randomization | | X | | | | | | | | | | | | | | | | |
| via | | | | | | | | | | | | | | | | | | |
| IWRS | | | | | | | | | | | | | | | | | | |
| Dispense | | X | X | X | X | X | X | X | X | X | X | X | X | | | | | Participants |
| study | | | | | | | | | | | | | | | | | | will self- |
| intervention | | | | | | | | | | | | | | | | | | administer |
| to | | | | | | | | | | | | | | | | | | study |
| participant | | | | | | | | | | | | | | | | | | intervention |
| via IWRS | | | | | | | | | | | | | | | | | | at home, |
| | | | | | | | | | | | | | | | | | | except for |
| | | | | | | | | | | | | | | | | | | the day of |
| | | | | | | | | | | | | | | | | | | any study |
| | | | | | | | | | | | | | | | | | | visit. |
| | | | | | | | | | | | | | | | | | | Starting at |
| | | | | | | | | | | | | | | | | | | Week 14, |
| | | | | | | | | | | | | | | | | | | applicable |
| | | | | | | | | | | | | | | | | | | only to |
| | | | | | | | | | | | | | | | | | | participants |
| | | | | | | | | | | | | | | | | | | who |
| | | | | | | | | | | | | | | | | | | continue |
| | | | | | | | | | | | | | | | | | | receiving the |
| | | | | | | | | | | | | | | | | | | study |
| | | | | | | | | | | | | | | | | | | intervention |
| | | | | | | | | | | | | | | | | | | (Section |
| | | | | | | | | | | | | | | | | | | 6.1.1). |
| Observe | | X | X | X | | X | | X | | X | | X | | | | | | Participants |
| participant | | | | | | | | | | | | | | | | | | will be |
| administer | | | | | | | | | | | | | | | | | | instructed to |
| study | | | | | | | | | | | | | | | | | | withhold |
| intervention | | | | | | | | | | | | | | | | | | study |
| | | | | | | | | | | | | | | | | | | intervention |
| | | | | | | | | | | | | | | | | | | on study |
| | | | | | | | | | | | | | | | | | | visit days, |
| | | | | | | | | | | | | | | | | | | until after |
| | | | | | | | | | | | | | | | | | | pre-dose |
| | | | | | | | | | | | | | | | | | | laboratory |
| | | | | | | | | | | | | | | | | | | samples |
| | | | | | | | | | | | | | | | | | | have been |
| | | | | | | | | | | | | | | | | | | collected. |
| | | | | | | | | | | | | | | | | | | Starting at |
| | | | | | | | | | | | | | | | | | | Week 16, |
| | | | | | | | | | | | | | | | | | | applicable |
| | | | | | | | | | | | | | | | | | | only to |
| | | | | | | | | | | | | | | | | | | participants |
| | | | | | | | | | | | | | | | | | | who |
| | | | | | | | | | | | | | | | | | | continue |
| | | | | | | | | | | | | | | | | | | receiving the |
| | | | | | | | | | | | | | | | | | | study |
| | | | | | | | | | | | | | | | | | | intervention |
| | | | | | | | | | | | | | | | | | | (Section |
| | | | | | | | | | | | | | | | | | | 6.1.1). |
| Participant | | | X | X | X | X | X | X | X | X | X | X | X | X | X | | | Participant |
| returns study | | | | | | | | | | | | | | | | | | is expected |
| intervention | | | | | | | | | | | | | | | | | | to bring |
| | | | | | | | | | | | | | | | | | | unused |
| | | | | | | | | | | | | | | | | | | intervention |
| | | | | | | | | | | | | | | | | | | back to the |
| | | | | | | | | | | | | | | | | | | clinic. |
| | | | | | | | | | | | | | | | | | | Starting at |
| | | | | | | | | | | | | | | | | | | Week 16, |
| | | | | | | | | | | | | | | | | | | applicable |
| | | | | | | | | | | | | | | | | | | only to |
| | | | | | | | | | | | | | | | | | | participants |
| | | | | | | | | | | | | | | | | | | who |
| | | | | | | | | | | | | | | | | | | continue |
| | | | | | | | | | | | | | | | | | | receiving the |
| | | | | | | | | | | | | | | | | | | study |
| | | | | | | | | | | | | | | | | | | intervention |
| | | | | | | | | | | | | | | | | | | (Section |
| | | | | | | | | | | | | | | | | | | 6.1.1). |
| Assess study | | | X | X | X | X | X | X | X | X | X | X | X | X | X | | | Starting at |
| intervention | | | | | | | | | | | | | | | | | | Week 16, |
| compliance | | | | | | | | | | | | | | | | | | applicable |
| | | | | | | | | | | | | | | | | | | only to |
| | | | | | | | | | | | | | | | | | | participants |
| | | | | | | | | | | | | | | | | | | who |
| | | | | | | | | | | | | | | | | | | continue |
| | | | | | | | | | | | | | | | | | | receiving the |
| | | | | | | | | | | | | | | | | | | study |
| | | | | | | | | | | | | | | | | | | intervention |
| | | | | | | | | | | | | | | | | | | (Section |
| | | | | | | | | | | | | | | | | | | 6.1.1). |

Schedule of Activities for Phase 2b of This Study
Visit 1 procedures may be conducted over more than 1 day as long as all activities are completed within the allowable visit tolerance.
For early discontinuations that occur before the last visit in the treatment period, see the activities listed for ED in this table.

Schedule of Activities for Phase 2b of This Study

| | | | | | | | | | | | | Period | | |
|---|---|---|---|---|---|---|---|---|---|---|---|---|---|---|
| | | | | | | | | | | | | IV | | |
| | Period | Period II | | | | | | | | | | Post- | | |
| | I | Double-Blind, Placebo- | | | | | Period III | | | | | Treatment | | |
| | Screening | Controlled Treatment | | | | | Treatment | | | | | Follow-up | | |
| Visit number | 1 | 2 | 103 | 104 | 105 | 106 | 107 | 108 | 109 | 110 | ED | 801 | 802 | Comments |
| Weeks from | — | — | 2 | 4 | 8 | 12 | 14 | 16 | 20 | 24 | — | 28 | 32 | V801 occurs 4 |
| randomization | | | | | | | | | | | | | | weeks after |
| | | | | | | | | | | | | | | V110 or ED. |
| | | | | | | | | | | | | | | V802 occurs 8 |
| | | | | | | | | | | | | | | weeks after |
| | | | | | | | | | | | | | | V110 or ED. |
| Visit interval | −42 to | — | ±4 | ±4 | ±4 | ±4 | ±4 | ±4 | ±4 | ±4 | — | ±7 | ±7 | |
| tolerance | −1 | | | | | | | | | | | | | |
| (days) | | | | | | | | | | | | | | |
| Fasting visit | X | X | | | | X | | | | X | X | X | X | See “Lipid |
| | | | | | | | | | | | | | | panel” row for |
| | | | | | | | | | | | | | | details. |
| Informed | X | | | | | | | | | | | | | The ICF must |
| consent | | | | | | | | | | | | | | be signed |
| | | | | | | | | | | | | | | before any |
| | | | | | | | | | | | | | | protocol- |
| | | | | | | | | | | | | | | specific |
| | | | | | | | | | | | | | | tests/procedures |
| | | | | | | | | | | | | | | are |
| | | | | | | | | | | | | | | performed. |
| | | | | | | | | | | | | | | See Appendix |
| | | | | | | | | | | | | | | 10.1, Section |
| | | | | | | | | | | | | | | 10.1.3, for |
| | | | | | | | | | | | | | | additional |
| | | | | | | | | | | | | | | details. |
| Inclusion and | X | X | | | | | | | | | | | | Confirm |
| exclusion | | | | | | | | | | | | | | inclusion/ |
| criteria, review | | | | | | | | | | | | | | exclusion criteria |
| and confirm | | | | | | | | | | | | | | before |
| | | | | | | | | | | | | | | assignment and |
| | | | | | | | | | | | | | | administration |
| | | | | | | | | | | | | | | of first dose of |
| | | | | | | | | | | | | | | study |
| | | | | | | | | | | | | | | intervention. |
| Demographics | X | | | | | | | | | | | | | Includes |
| | | | | | | | | | | | | | | ethnicity |
| | | | | | | | | | | | | | | (United States |
| | | | | | | | | | | | | | | only), year of |
| | | | | | | | | | | | | | | birth, sex, and |
| | | | | | | | | | | | | | | race (as |
| | | | | | | | | | | | | | | allowed per |
| | | | | | | | | | | | | | | local |
| | | | | | | | | | | | | | | regulations). |
| Preexisting | X | | | | | | | | | | | | | Collect all |
| conditions and | | | | | | | | | | | | | | ongoing |
| medical | | | | | | | | | | | | | | conditions and |
| history, | | | | | | | | | | | | | | relevant past |
| including | | | | | | | | | | | | | | surgical and |
| relevant | | | | | | | | | | | | | | medical |
| surgical | | | | | | | | | | | | | | history. |
| history | | | | | | | | | | | | | | |
| Prespecified | X | | | | | | | | | | | | | Includes RA |
| medical | | | | | | | | | | | | | | diagnosis and |
| history | | | | | | | | | | | | | | onset and prior |
| (indication and | | | | | | | | | | | | | | malignancy. |
| history of | | | | | | | | | | | | | | |
| interest) | | | | | | | | | | | | | | |
| Prior | X | | | | | | | | | | | | | Any previous |
| treatments for | | | | | | | | | | | | | | therapy for RA |
| indication | | | | | | | | | | | | | | in the past 15 |
| | | | | | | | | | | | | | | years that has |
| | | | | | | | | | | | | | | now been |
| | | | | | | | | | | | | | | discontinued. |
| Substance use | X | | | | | | | | | | | | | |
| (alcohol, | | | | | | | | | | | | | | |
| caffeine, | | | | | | | | | | | | | | |
| tobacco use) | | | | | | | | | | | | | | |
| Concomitant | X | X | X | X | X | X | X | X | X | X | X | X | X | Collect |
| medications | | | | | | | | | | | | | | additional data |
| | | | | | | | | | | | | | | for |
| | | | | | | | | | | | | | | concomitant |
| | | | | | | | | | | | | | | medications of |
| | | | | | | | | | | | | | | interest (that is, |
| | | | | | | | | | | | | | | permitted |
| | | | | | | | | | | | | | | therapies for |
| | | | | | | | | | | | | | | RA; |
| | | | | | | | | | | | | | | Sections 6.9.1 |
| | | | | | | | | | | | | | | and 6.9.2). |
| Adverse events | X | X | X | X | X | X | X | X | X | X | X | X | X | Any events |
| (AEs) | | | | | | | | | | | | | | that occur after |
| | | | | | | | | | | | | | | signing the |
| | | | | | | | | | | | | | | ICF are |
| | | | | | | | | | | | | | | considered |
| | | | | | | | | | | | | | | AEs as defined |
| | | | | | | | | | | | | | | in |
| | | | | | | | | | | | | | | Section 8.3.1. |
| | | | | | | | | | | | | | | For AESIs, |
| | | | | | | | | | | | | | | collect |
| | | | | | | | | | | | | | | additional data |
| | | | | | | | | | | | | | | (Section 8.3.3). |

Physical
evaluation

| Height | X | | | | | | | | | | | | | Participant |
|---|---|---|---|---|---|---|---|---|---|---|---|---|---|---|
| | | | | | | | | | | | | | | should remove |
| | | | | | | | | | | | | | | shoes. |
| Weight | X | X | | | | X | | | | | X | X | X | |
| Vital signs | X | X | X | X | X | X | X | X | X | X | X | X | X | Includes blood |
| | | | | | | | | | | | | | | pressure, body |
| | | | | | | | | | | | | | | temperature, |
| | | | | | | | | | | | | | | and pulse rate. |
| | | | | | | | | | | | | | | Measure vital |
| | | | | | | | | | | | | | | signs after |
| | | | | | | | | | | | | | | participant has |
| | | | | | | | | | | | | | | been sitting at |
| | | | | | | | | | | | | | | least 5 |
| | | | | | | | | | | | | | | minutes, |
| | | | | | | | | | | | | | | before |
| | | | | | | | | | | | | | | obtaining an |
| | | | | | | | | | | | | | | ECG tracing, |
| | | | | | | | | | | | | | | and before |
| | | | | | | | | | | | | | | collection of |
| | | | | | | | | | | | | | | blood samples |
| | | | | | | | | | | | | | | for laboratory |
| | | | | | | | | | | | | | | testing. |
| Physical | X | | | | | X | | | | | X | | X | See Section |
| examination | | | | | | | | | | | | | | 8.2.1. |
| | | | | | | | | | | | | | | The complete |
| | | | | | | | | | | | | | | physical |
| | | | | | | | | | | | | | | examination |
| | | | | | | | | | | | | | | excludes |
| | | | | | | | | | | | | | | pelvic, rectal, |
| | | | | | | | | | | | | | | and breast |
| | | | | | | | | | | | | | | examinations, |
| | | | | | | | | | | | | | | unless |
| | | | | | | | | | | | | | | clinically |
| | | | | | | | | | | | | | | indicated. |
| Symptom- | | X | | | | | | | | | X | X | | See Section |
| directed | | | | | | | | | | | | | | 8.2.1. |
| physical | | | | | | | | | | | | | | Conduct at the |
| assessment | | | | | | | | | | | | | | discretion of |
| | | | | | | | | | | | | | | the investigator |
| | | | | | | | | | | | | | | or qualified |
| | | | | | | | | | | | | | | personnel per |
| | | | | | | | | | | | | | | local |
| | | | | | | | | | | | | | | regulations, as |
| | | | | | | | | | | | | | | indicated based |
| | | | | | | | | | | | | | | on participant |
| | | | | | | | | | | | | | | status and |
| | | | | | | | | | | | | | | standard of |
| | | | | | | | | | | | | | | care. |
| Tuberculosis | | | | | | X | | | | X | X | | X | See Sections |
| (TB) | | | | | | | | | | | | | | 8.2.1 and 8.2.9. |
| monitoring and | | | | | | | | | | | | | | |
| thyroid | | | | | | | | | | | | | | |
| palpation | | | | | | | | | | | | | | |
| 12-lead ECG | X | X | X | X | X | X | X | X | X | X | X | X | X | ECGs should |
| (centrally read | | | | | | | | | | | | | | be recorded |
| and locally | | | | | | | | | | | | | | prior to |
| interpretated) | | | | | | | | | | | | | | collection of |
| | | | | | | | | | | | | | | blood samples |
| | | | | | | | | | | | | | | for any |
| | | | | | | | | | | | | | | laboratory |
| | | | | | | | | | | | | | | testing. Local |
| | | | | | | | | | | | | | | ECGs may be |
| | | | | | | | | | | | | | | obtained at |
| | | | | | | | | | | | | | | additional |
| | | | | | | | | | | | | | | times, when |
| | | | | | | | | | | | | | | deemed |
| | | | | | | | | | | | | | | clinically |
| | | | | | | | | | | | | | | necessary. |
| | | | | | | | | | | | | | | See Section |
| | | | | | | | | | | | | | | 8.2.3. |
| Chest x-ray | X | | | | | | | | | | | | | See Section |
| (posterior- | | | | | | | | | | | | | | 8.2.4. |
| anterior and | | | | | | | | | | | | | | Not done at |
| lateral view; | | | | | | | | | | | | | | screening if |
| local) | | | | | | | | | | | | | | done within |
| | | | | | | | | | | | | | | 3 months |
| | | | | | | | | | | | | | | before |
| | | | | | | | | | | | | | | screening, |
| | | | | | | | | | | | | | | and |
| | | | | | | | | | | | | | | documentation |
| | | | | | | | | | | | | | | of the |
| | | | | | | | | | | | | | | previous |
| | | | | | | | | | | | | | | chest X-ray |
| | | | | | | | | | | | | | | is available. |
| | | | | | | | | | | | | | | Interpreted and |
| | | | | | | | | | | | | | | reported by |
| | | | | | | | | | | | | | | radiologist or |
| | | | | | | | | | | | | | | pulmonologist. |
| Schedule MRI | X | | | | X | | | | | | | | | If not |
| | | | | | | | | | | | | | | completed at |
| | | | | | | | | | | | | | | V105, this will |
| | | | | | | | | | | | | | | not be |
| | | | | | | | | | | | | | | considered a |
| | | | | | | | | | | | | | | protocol |
| | | | | | | | | | | | | | | deviation. |
| MRI | X | | | | | X | | | | | | | | At screening, |
| (hand/wrist) | | | | | | | | | | | | | | perform MRI |
| | | | | | | | | | | | | | | only after all |
| | | | | | | | | | | | | | | eligibility |
| | | | | | | | | | | | | | | criteria are met |
| | | | | | | | | | | | | | | except |
| | | | | | | | | | | | | | | Inclusion |
| | | | | | | | | | | | | | | Criterion [8] |
| | | | | | | | | | | | | | | (exception can |
| | | | | | | | | | | | | | | be considered |
| | | | | | | | | | | | | | | after |
| | | | | | | | | | | | | | | consultation |
| | | | | | | | | | | | | | | with the |
| | | | | | | | | | | | | | | sponsor). |
| | | | | | | | | | | | | | | Evaluate the |
| | | | | | | | | | | | | | | same |
| | | | | | | | | | | | | | | hand/wrist at |
| | | | | | | | | | | | | | | screening and |
| | | | | | | | | | | | | | | at Week 12. |
| | | | | | | | | | | | | | | At Week 12, |
| | | | | | | | | | | | | | | MRI may be |
| | | | | | | | | | | | | | | completed ±7 |
| | | | | | | | | | | | | | | days of the |
| | | | | | | | | | | | | | | planned Week |
| | | | | | | | | | | | | | | 12 visit. |
| | | | | | | | | | | | | | | Complete |
| | | | | | | | | | | | | | | before any |
| | | | | | | | | | | | | | | clinician- |
| | | | | | | | | | | | | | | administered |
| | | | | | | | | | | | | | | assessments |
| | | | | | | | | | | | | | | and dosing, in |
| | | | | | | | | | | | | | | the order listed |
| Patient-Reported Outcomes (Electronic) | | | | | | | | | | | | | | here. |
| Patient's | X | X | X | X | X | X | X | X | X | X | X | X | X | |
| Global | | | | | | | | | | | | | | |
| Assessment of | | | | | | | | | | | | | | |
| Arthritis Pain | | | | | | | | | | | | | | |
| (VAS) | | | | | | | | | | | | | | |
| Health | X | X | X | X | X | X | X | X | X | X | X | X | X | |
| Assessment | | | | | | | | | | | | | | |
| Questionnaire- | | | | | | | | | | | | | | |
| Disability | | | | | | | | | | | | | | |
| Index (HAQ- | | | | | | | | | | | | | | |
| DI) | | | | | | | | | | | | | | |
| Patient's | X | X | X | X | X | X | X | X | X | X | X | X | X | |
| Global | | | | | | | | | | | | | | |
| Assessment of | | | | | | | | | | | | | | |
| Disease | | | | | | | | | | | | | | |
| Activity (RA) | | | | | | | | | | | | | | |
| (PaGADA_V | | | | | | | | | | | | | | |
| AS) | | | | | | | | | | | | | | |
| Medical | | X | | | | X | | | | X | X | | X | |
| Outcomes | | | | | | | | | | | | | | |
| Study 36-Item | | | | | | | | | | | | | | |
| Short Form | | | | | | | | | | | | | | |
| Health Survey | | | | | | | | | | | | | | |
| (SF-36 | | | | | | | | | | | | | | |
| v2.Acute) | | | | | | | | | | | | | | |
| Morning Joint | | X | X | X | X | X | X | X | X | X | X | X | X | |
| Stiffness | | | | | | | | | | | | | | |
| Duration PRO | | | | | | | | | | | | | | |
| (24-hr recall) | | | | | | | | | | | | | | |
| Morning Joint | | X | X | X | X | X | X | X | X | X | X | X | X | |
| Stiffness | | | | | | | | | | | | | | |
| Severity | | | | | | | | | | | | | | |
| (NRS) (day of | | | | | | | | | | | | | | |
| visit) | | | | | | | | | | | | | | |
| FACIT-F | | X | X | X | X | X | X | X | X | X | X | X | X | |
| Clinician-Administered | | | | | | | | | | | | | | Determine |
| Assessments (Electronic) | | | | | | | | | | | | | | before dosing. |
| Clinician- | | X | X | X | X | X | X | X | X | X | X | X | X | The Joint |
| Administered | | | | | | | | | | | | | | Assessor will |
| Tender/Swollen | | | | | | | | | | | | | | perform the |
| Joint Count | | | | | | | | | | | | | | joint counts. |
| (TSJC) (68/66) | | | | | | | | | | | | | | See Section 8. |
| | | | | | | | | | | | | | | If a participant |
| | | | | | | | | | | | | | | receives intra- |
| | | | | | | | | | | | | | | articular joint |
| | | | | | | | | | | | | | | injections as |
| | | | | | | | | | | | | | | standard-of- |
| | | | | | | | | | | | | | | care therapy |
| | | | | | | | | | | | | | | (Section 6.1.1), |
| | | | | | | | | | | | | | | record the |
| | | | | | | | | | | | | | | treated joints |
| | | | | | | | | | | | | | | as “not |
| | | | | | | | | | | | | | | evaluable” on |
| | | | | | | | | | | | | | | subsequent |
| | | | | | | | | | | | | | | assessments |
| | | | | | | | | | | | | | | for the |
| | | | | | | | | | | | | | | remainder of |
| | | | | | | | | | | | | | | the study. |
| Physician | X | X | X | X | X | X | X | X | X | X | X | X | X | |
| Global | | | | | | | | | | | | | | |
| Assessment of | | | | | | | | | | | | | | |
| Disease | | | | | | | | | | | | | | |
| Activity | | | | | | | | | | | | | | |
| (PhGADA) | | | | | | | | | | | | | | |

Clinician-Administered
Assessments (Paper)

| | | | | | | | | | | | | | | |
|---|---|---|---|---|---|---|---|---|---|---|---|---|---|---|
| C-SSRS | X | | | | | | | | | | | | | Collect AEs |
| Screening/ | | | | | | | | | | | | | | before the |
| Baseline | | | | | | | | | | | | | | collection of |
| C-SSRS Since | | X | X | X | X | X | X | X | X | X | X | X | X | the C-SSRS. |
| Last Assessed | | | | | | | | | | | | | | |

Actigraphy

| Actigraphy | X | | | | X | | | | X | | | | | See Appendix |
|---|---|---|---|---|---|---|---|---|---|---|---|---|---|---|
| device | | | | | | | | | | | | | | 10.9. |
| dispense | | | | | | | | | | | | | | |
| Actigraphy | | X | | | | X | | | | X | X | | | Participants |
| device return | | | | | | | | | | | | | | not eligible for |
| | | | | | | | | | | | | | | the study |
| | | | | | | | | | | | | | | should return |
| | | | | | | | | | | | | | | the device |
| | | | | | | | | | | | | | | upon screen |
| | | | | | | | | | | | | | | failure. A |
| | | | | | | | | | | | | | | minimum of |
| | | | | | | | | | | | | | | 14 continuous |
| | | | | | | | | | | | | | | days of |
| | | | | | | | | | | | | | | actigraphy data |
| | | | | | | | | | | | | | | should be |
| | | | | | | | | | | | | | | collected |
| | | | | | | | | | | | | | | before the |
| | | | | | | | | | | | | | | device is |
| | | | | | | | | | | | | | | returned. |
| | | | | | | | | | | | | | | However, |
| | | | | | | | | | | | | | | collection of |
| | | | | | | | | | | | | | | less than |
| | | | | | | | | | | | | | | 14 continuous |
| | | | | | | | | | | | | | | days of data |
| | | | | | | | | | | | | | | will not be |
| | | | | | | | | | | | | | | considered a |
| | | | | | | | | | | | | | | protocol |
| | | | | | | | | | | | | | | deviation. See |
| | | | | | | | | | | | | | | Appendix 10.9. |
| | | | | | | | | | | | | | | See Appendix |
| | | | | | | | | | | | | | | 10.2 for details |
| | | | | | | | | | | | | | | of testing |
| | | | | | | | | | | | | | | included in |
| Laboratory Tests and Sample Collections | | | | | | | | | | | | | | panels. |
| Hematology | X | X | X | X | X | X | X | X | X | X | X | X | X | Collect pre- |
| | | | | | | | | | | | | | | dose. |
| Erythrocyte | X | X | X | X | X | X | X | X | X | X | X | X | X | Collect pre- |
| sedimentation | | | | | | | | | | | | | | dose. |
| rate (ESR) | | | | | | | | | | | | | | |
| (local) | | | | | | | | | | | | | | |
| Clinical | X | X | X | X | X | X | X | X | X | X | X | X | X | Collect pre- |
| chemistry | | | | | | | | | | | | | | dose. |
| Lipid panel | X | X | | | | X | | | | X | X | X | X | Collect pre- |
| | | | | | | | | | | | | | | dose. |
| | | | | | | | | | | | | | | Participants |
| | | | | | | | | | | | | | | should not eat |
| | | | | | | | | | | | | | | or drink |
| | | | | | | | | | | | | | | anything but |
| | | | | | | | | | | | | | | water for 9-12 |
| | | | | | | | | | | | | | | hours before |
| | | | | | | | | | | | | | | the visit. |
| | | | | | | | | | | | | | | Fasting is not |
| | | | | | | | | | | | | | | required at an |
| | | | | | | | | | | | | | | ED visit. If a |
| | | | | | | | | | | | | | | participant |
| | | | | | | | | | | | | | | attends these |
| | | | | | | | | | | | | | | visits in a |
| | | | | | | | | | | | | | | nonfasting |
| | | | | | | | | | | | | | | state, the |
| | | | | | | | | | | | | | | sample should |
| | | | | | | | | | | | | | | still be |
| | | | | | | | | | | | | | | collected. This |
| | | | | | | | | | | | | | | will not be |
| | | | | | | | | | | | | | | considered a |
| | | | | | | | | | | | | | | protocol |
| | | | | | | | | | | | | | | deviation. |
| Thyroid panel | | X | | | | X | | | | X | X | X | X | Collect pre- |
| | | | | | | | | | | | | | | dose. |
| Coagulation | | X | | X | X | X | X | X | X | X | X | X | X | Collect pre- |
| | | | | | | | | | | | | | | dose. |
| ACTH | | X | | | | X | | | | X | | | X | Collect |
| | | | | | | | | | | | | | | between 8 am |
| | | | | | | | | | | | | | | and 10 am. |
| Cortisol | | X | | | | X | | | | X | | | X | Collect pre- |
| | | | | | | | | | | | | | | dose. |
| Renin | | X | | | | X | | | | X | | | X | Collect pre- |
| DHEA-S | | X | | | | X | | | | X | | | X | dose. |
| Testosterone | | X | | | | X | | | | X | | | X | For women |
| | | | | | | | | | | | | | | only. Collect |
| | | | | | | | | | | | | | | between 8 am |
| | | | | | | | | | | | | | | and 10 am. |
| | | | | | | | | | | | | | | Collect pre- |
| | | | | | | | | | | | | | | dose. |
| Urinalysis | X | X | | X | | X | | X | | X | X | X | | Collect pre- |
| | | | | | | | | | | | | | | dose. |
| Serum | X | | | | | | | | | | | | | Only for |
| pregnancy | | | | | | | | | | | | | | WOCBP. See |
| | | | | | | | | | | | | | | Appendix 10.4, |
| | | | | | | | | | | | | | | Sections 10.4.1 |
| | | | | | | | | | | | | | | and 10.4.2. |
| Urine | | X | | X | X | X | | X | X | X | X | X | X | Only for |
| pregnancy | | | | | | | | | | | | | | WOCBP. See |
| (local) | | | | | | | | | | | | | | Section 8.2.6 |
| | | | | | | | | | | | | | | and |
| | | | | | | | | | | | | | | Appendix 10.4. |
| Follicle- | X | | | | | | | | | | | | | Optional; |
| stimulating | | | | | | | | | | | | | | perform only |
| hormone | | | | | | | | | | | | | | to confirm |
| (FSH) | | | | | | | | | | | | | | postmenopausal |
| | | | | | | | | | | | | | | status. |
| C-reactive | X | X | X | X | X | X | X | X | X | X | X | X | X | Collect pre- |
| protein, high- | | | | | | | | | | | | | | dose. |
| sensitivity | | | | | | | | | | | | | | |
| (hsCRP) | | | | | | | | | | | | | | |
| Rheumatoid | X | X | | | | X | | | | X | X | | X | Collect pre- |
| factor (RF) | | | | | | | | | | | | | | dose. |
| Anticyclic | X | X | | | | X | | | | X | X | | X | Collect pre- |
| citrullinated | | | | | | | | | | | | | | dose. |
| peptide (anti- | | | | | | | | | | | | | | |
| CCP) | | | | | | | | | | | | | | |
| Flow | | X | | | | X | | | | X | X | | X | |
| cytometry | | | | | | | | | | | | | | |
| panel | | | | | | | | | | | | | | |
| Immunoglobulin | | X | | | | X | | | | X | X | | X | Collect |
| panel | | | | | | | | | | | | | | samples pre- |
| Protein disease | | X | | X | X | X | | | | X | X | | X | dose. |
| activity (DA) | | | | | | | | | | | | | | |
| panel | | | | | | | | | | | | | | |
| Troponin | | X | | | | X | X | | | X | X | | X | |
| TB test | X | | | | | | | | | | | | | See Appendix |
| | | | | | | | | | | | | | | 10.2. |
| | | | | | | | | | | | | | | Participants |
| | | | | | | | | | | | | | | who had a |
| | | | | | | | | | | | | | | tuberculin skin |
| | | | | | | | | | | | | | | test (TST) |
| | | | | | | | | | | | | | | must have the |
| | | | | | | | | | | | | | | test read 48 to |
| | | | | | | | | | | | | | | 72 hours after |
| | | | | | | | | | | | | | | placement. The |
| | | | | | | | | | | | | | | TST test does |
| | | | | | | | | | | | | | | not need to be |
| | | | | | | | | | | | | | | read at the site |
| | | | | | | | | | | | | | | but must be |
| | | | | | | | | | | | | | | read by a |
| | | | | | | | | | | | | | | trained |
| | | | | | | | | | | | | | | professional |
| | | | | | | | | | | | | | | and results |
| | | | | | | | | | | | | | | must be |
| | | | | | | | | | | | | | | presented to |
| | | | | | | | | | | | | | | the site prior to |
| | | | | | | | | | | | | | | first dose of |
| | | | | | | | | | | | | | | study |
| | | | | | | | | | | | | | | intervention. |
| | | | | | | | | | | | | | | See Section |
| | | | | | | | | | | | | | | 8.2.9 and |
| | | | | | | | | | | | | | | Appendix 10.7. |
| HIV screening | X | | | | | | | | | | | | | |
| tests | | | | | | | | | | | | | | |
| Hepatitis C | X | | | | | | | | | | | | | If HCV |
| virus (HCV) | | | | | | | | | | | | | | antibody test is |
| screening tests | | | | | | | | | | | | | | positive, it |
| | | | | | | | | | | | | | | must be |
| | | | | | | | | | | | | | | followed by an |
| | | | | | | | | | | | | | | HCV RNA |
| | | | | | | | | | | | | | | test. See |
| | | | | | | | | | | | | | | Section 8.2.11. |
| Hepatitis B | X | | | | | | | | | | | | | See |
| virus (HBV) | | | | | | | | | | | | | | Section 8.2.10 |
| screening tests | | | | | | | | | | | | | | and |
| | | | | | | | | | | | | | | Appendix 10.2. |
| Hepatitis B | X | | | | | X | | | | X | X | | X | Only for |
| virus (HBV) | | | | | | | | | | | | | | participants |
| DNA | | | | | | | | | | | | | | who are |
| | | | | | | | | | | | | | | positive for |
| | | | | | | | | | | | | | | anti-HBc at |
| | | | | | | | | | | | | | | screening. See |
| | | | | | | | | | | | | | | Section 8.2.10. |
| Estimated | X | | | | | | | | | | | | | Calculated |
| glomerular | | | | | | | | | | | | | | using the |
| filtration rate | | | | | | | | | | | | | | CKD-EPI |
| (eGFR) | | | | | | | | | | | | | | creatinine |
| | | | | | | | | | | | | | | equation |
| | | | | | | | | | | | | | | (2021). |
| Pharmacokinetic | | | | | | | | | | | | | | Collect PK |
| (PK) | | | | | | | | | | | | | | samples at |
| samples | | | | | | | | | | | | | | these times: |
| | | | | | | | | | | | | | | Visit 2: 1 to 3 |
| | | | | | | | | | | | | | | hours post- |
| | | | | | | | | | | | | | | dose. |
| | | | | | | | | | | | | | | Visits 103 and |
| | | | | | | | | | | | | | | V104: pre-dose |
| | | | | | | | | | | | | | | and 1 to 3 |
| | | | | | | | | | | | | | | hours post- |
| | | | | | | | | | | | | | | dose. |
| | | | | | | | | | | | | | | Visits 105-108: |
| | | | | | | | | | | | | | | pre-dose. |

Stored
Samples

| | | | | | | | | |
|---|---|---|---|---|---|---|---|---|
| Genetics | X | | | | | | | Sample can be |
| sample | | | | | | | | obtained at or |
| | | | | | | | | after the |
| | | | | | | | | specified visit. |
| Exploratory | X | X | X | X | X | X | X | Collect |
| biomarker | | | | | | | | samples pre- |
| samples | | | | | | | | dose. |

Randomization
and Dosing

| | | | | | | | | | | | | | | |
|---|---|---|---|---|---|---|---|---|---|---|---|---|---|---|
| Register visit | X | X | X | X | X | X | X | X | X | X | X | X | X | In the event of |
| with IWRS | | | | | | | | | | | | | | an emergency |
| | | | | | | | | | | | | | | unblinding, do |
| | | | | | | | | | | | | | | not register the |
| | | | | | | | | | | | | | | visit in IWRS. |
| Randomization | | X | | | | | | | | | | | | |
| via IWRS | | | | | | | | | | | | | | |
| Dispense study | | X | | X | X | X | X | X | X | | | | | Participants |
| intervention to | | | | | | | | | | | | | | will self- |
| participant via | | | | | | | | | | | | | | administer |
| IWRS | | | | | | | | | | | | | | study |
| | | | | | | | | | | | | | | intervention at |
| | | | | | | | | | | | | | | home, except |
| | | | | | | | | | | | | | | for the day of |
| | | | | | | | | | | | | | | any study visit. |
| | | | | | | | | | | | | | | Starting at |
| | | | | | | | | | | | | | | Week 14, |
| | | | | | | | | | | | | | | applicable only |
| | | | | | | | | | | | | | | to participants |
| | | | | | | | | | | | | | | who continue |
| | | | | | | | | | | | | | | receiving the |
| | | | | | | | | | | | | | | study |
| | | | | | | | | | | | | | | intervention |
| | | | | | | | | | | | | | | (Section 6.1.1). |
| Observe | | X | X | X | X | X | | X | X | | | | | Participants |
| participant | | | | | | | | | | | | | | will be |
| administer | | | | | | | | | | | | | | instructed to |
| study | | | | | | | | | | | | | | withhold study |
| intervention | | | | | | | | | | | | | | intervention on |
| | | | | | | | | | | | | | | study visit |
| | | | | | | | | | | | | | | days, until |
| | | | | | | | | | | | | | | after pre-dose |
| | | | | | | | | | | | | | | laboratory |
| | | | | | | | | | | | | | | samples have |
| | | | | | | | | | | | | | | been collected. |
| | | | | | | | | | | | | | | Starting at |
| | | | | | | | | | | | | | | Week 16, |
| | | | | | | | | | | | | | | applicable only |
| | | | | | | | | | | | | | | to participants |
| | | | | | | | | | | | | | | who continue |
| | | | | | | | | | | | | | | receiving the |
| | | | | | | | | | | | | | | study |
| | | | | | | | | | | | | | | intervention |
| | | | | | | | | | | | | | | (Section 6.1.1). |
| Participant | | | X | X | X | X | X | X | X | X | X | | | Participant is |
| returns study | | | | | | | | | | | | | | expected to |
| intervention | | | | | | | | | | | | | | bring unused |
| | | | | | | | | | | | | | | intervention |
| | | | | | | | | | | | | | | back to the |
| | | | | | | | | | | | | | | clinic. |
| | | | | | | | | | | | | | | Starting at |
| | | | | | | | | | | | | | | Week 16, |
| | | | | | | | | | | | | | | applicable only |
| | | | | | | | | | | | | | | to participants |
| | | | | | | | | | | | | | | who continue |
| | | | | | | | | | | | | | | receiving the |
| | | | | | | | | | | | | | | study |
| | | | | | | | | | | | | | | intervention |
| | | | | | | | | | | | | | | (Section 6.1.1). |
| Assess study | | | X | X | X | X | X | X | X | X | X | | | Starting at |
| intervention | | | | | | | | | | | | | | Week 16, |
| compliance | | | | | | | | | | | | | | applicable only |
| | | | | | | | | | | | | | | to participants |
| | | | | | | | | | | | | | | who continue |
| | | | | | | | | | | | | | | receiving the |
| | | | | | | | | | | | | | | study |
| | | | | | | | | | | | | | | intervention |
| | | | | | | | | | | | | | | (Section 6.1.1). |

Abbreviations: ACTH = adrenocorticotropic hormone; AESI = adverse event of special interest; anti-HBc = hepatitis B core antibody; CKD-EPI = Chronic Kidney Disease Epidemiology Collaboration; C-SSRS = Columbia Suicide-Severity Rating Scale; DHEA-S = dehydroepiandrosterone sulfate; ECG = electrocardiogram; ED = early discontinuation; FACIT-F = Functional Assessment of Chronic Illness Therapy-Fatigue; HIV = human immunodeficiency virus; ICF = informed consent form; IWRS = interactive web-response system; MRI = magnetic resonance imaging; NRS = numeric rating scale; PRO = patient-reported outcome; RA = rheumatoid arthritis; VAS = visual analog scale; WOCBP = women of childbearing potential.

### Objectives, Endpoints, and Estimands

Unless specified otherwise, the objectives and endpoints in this table apply to both Phase 2a and Phase 2b of this study.

| Objectives | Endpoints |
|---|---|
| Primary | |
| Phase 2a | |
| To evaluate the efficacy of Compound I | Change in DAS28-hsCRP from baseline |
| compared to placebo as measured by changes | to Week 12 |
| in DAS28-hsCRP | |
| Phase 2b | |
| To evaluate the efficacy of Compound I | Proportion of participants achieving |
| compared to placebo in achieving ACR50 | ACR50 at Week 12 |
| Secondary | |
| To evaluate the effect of Compound I | Phase 2a: Proportions of participants |
| compared to placebo for measures of disease | achieving ACR20/50/70 at Week 12 |
| activity | Phase 2b: |
| | Change in DAS28-hsCRP from |
| | baseline to Week 12 |
| | Proportions of participants achieving |
| | ACR20/70 at Week 12 |
| | Changes from baseline to Week 12 for |
| | mean |
| | SDAI |
| | CDAI |
| | Change from baseline to Week 12 ACR |
| | core set values |
| | 68 tender joint counts |
| | 66 swollen joint counts, and |
| | PhGADA_VAS |
| To evaluate the effect of Compound I | Change from baseline to Week 12 for |
| compared to placebo for patient-reported | patient-reported ACR core set values |
| outcome measures | oPaGADA_VAS |
| | Patient's Assessment of Arthritis Pain |
| | (VAS), and |
| | patient's assessment of physical |
| | function using HAQ-DI |
| | Change from baseline to Week 12 for |
| | the duration and severity of morning |
| | joint stiffness |
| | FACIT-F scores |
| | Change from baseline at Week 12 for |
| | SF-36 domains |
| | SF-36 Physical Component |
| | Summary, and |
| | SF-36 Mental Component Summary |
| To assess clinical disease activity following | Proportion of participants achieving LDA |
| treatment with Compound I | or remission at Week 24 for |
| | SDAI, and |
| | CDAI |
| | Proportion of participants achieving |
| | ACR20/50/70 at Week 24 |
| | Change in DAS28-hsCRP from baseline |
| | to Week 24 |
| | Change from baseline at Week 24 for |
| | ACR core set values |
| | 68 tender joint count |
| | 66 swollen joint count, and |
| | PhGADA VAS |
| | Change from baseline at Week 24 for |
| | mean |
| | SDAI, and |
| | CDAI |
| To characterize the pharmacokinetics of | Trough plasma concentrations of |
| Compound I | Compound I |
| Exploratory | |
| To assess patient-reported outcome measures | Change from baseline at Week 24 for |
| following treatment with Compound I | ACR core set values |
| | PaGADA_VAS |
| | Patient's Assessment of Arthritis Pain |
| | (VAS), and |
| | patient's assessment of physical |
| | function using HAQ-DI |
| | Change from baseline at Week 24 for |
| | the duration and severity of morning |
| | joint stiffness |
| | FACIT-F scale scores, and |
| | SF-36 quality of life measures |
| To evaluate the effect of Compound I | Change from baseline at Week 12 in the |
| compared to placebo on MRI measures of | RAMRIS inflammation and damage |
| disease activity | scores |
| To evaluate the durability of effect of | Proportion of participants that achieve |
| Compound I | and maintain LDA or remission at Week |
| | 12 through Week 24, measured by |
| | DAS28-hsCRP |
| | DAS28-ESR |
| | SDAI, and |
| | CDAI |
| | Proportion of participants that achieve |
| | LDA or remission at Week 12 or 24, |
| | measured by |
| | DAS28-hsCRP |
| | DAS28-ESR |
| | SDAI, and |
| | CDAI |
| To evaluate the effect of Compound I | Mean change from baseline to Week 12 in |
| compared to placebo on physical activity as | daily steps count |
| measured by an actigraphy device | daily average movement intensity |
| | average movement intensity in most |
| | active 10-hour window |
| To explore the relationship of Compound I | Percentage of participants that achieve |
| concentrations to clinical endpoints | the clinical endpoint at Weeks 12 or 24 |
| | (for example, ACR20 or ACR50) at |
| | different percentiles of exposure of |
| | Compound I |
| To explore the relationship of Compound I | Change from baseline to Week 12 or 24 |
| concentrations on biomarkers | in peripheral blood biomarkers |

Abbreviations: ACR = American College of Rheumatology; CDAI = Clinical Disease Activity Index; DAS28-ESR = Disease Activity Score-Erythrocyte Sedimentation Rate; DAS28-hsCRP = Disease Activity Score - high-sensitivity C-reactive protein; FACIT-F = Functional Assessment of Chronic Illness Therapy-Fatigue; HAQ-DI = Health Assessment Questionnaire - Disability Index; LDA = low disease activity; MRI = magnetic resonance imaging; PaGADA = Patient's Global Assessment of Disease Activity; PhGADA = Physician's Global Assessment of Disease Activity; RAMRIS = Rheumatoid Arthritis Magnetic Resonance Imaging Score; SDAI = Simplified Disease Activity Index; SF-36 = Short Form-36 version 2 health survey acute form; VAS = visual analog scale.

### Primary Estimands

### Phase 2a

The primary clinical question of interest is:
What is the difference between Compound I and placebo in the target patient population, in mean change from baseline at Week 12 if restrictions pertaining to changing permitted concomitant medications, and taking prohibited medications were not violated, and all participants adhered to the study intervention?

The estimand is described by the following attributes:
Population: Participants with moderately-to-severely active RA (see Section 5.1).
Endpoint: Change from baseline for DAS28-hsCRP
ICEs related to study intervention include the use of prohibited medication, the change in permitted concomitant medications, and early discontinuation from the study or study intervention. A hypothetical estimand strategy will be used; that is, data collected after the first occurrence of ICE will be excluded from an analysis and what the treatment effect would have been if prohibited medication was not used, permitted concomitant medications did not change, and all participants adhered to the treatment will be estimated.
Population-level summary: Difference in mean change from baseline to Week 12 between study intervention conditions.
Rationale for the estimand: The data collected after the treatment discontinuation, the initiation of prohibited medication, or the change in permitted concomitant medications will not present the true efficacy effects.

### Phase 2b

The primary clinical question of interest is:
What is the difference between Compound I and placebo in the target patient population, in achieving a successful response at Week 12 without changing permitted concomitant medications, taking prohibited medications, or discontinuing the study intervention?

The estimand is described by the following attributes:
Population: Participants with moderately-to-severely active RA (see Section 5.1).
Endpoint: ACR50 at Week 12
ICEs related to study intervention include the use of prohibited medication, the change in permitted concomitant medications, and early discontinuation from the study or study intervention. A composite strategy will be used; that is, a participant with ICEs will be considered as a nonresponder because it is assumed the participant was not receiving benefit from the intervention.
Population-level summary: Difference in proportion of participants achieving response at Week 12 between study intervention conditions.
Rationale for the estimand: The data collected after the treatment discontinuation, the initiation of prohibited medication, or the change in permitted concomitant medications will be categorized as treatment failures because it is assumed the participant was not receiving benefit from the intervention.

### Secondary Estimands

Unless specified otherwise, the information in this section applies to both phases of this study.

### Secondary Estimands for Categorical Endpoints

For secondary objectives that have categorical endpoints analyzed up to Week 12, the clinical question of interest is:
What is the difference between Compound I and placebo in the target patient population, in achieving a successful response at Week 12 without changing permitted concomitant medications, taking prohibited medications, or discontinuing the study intervention?

The estimand is described by the following attributes:
Population: Participants with moderately-to-severely active RA (see Section 5.1).
Endpoints: The following secondary endpoints
   ACR20
   ACR50 (for Phase 2a only), and
   ACR70.
ICE will be accounted using the same estimand strategy as for the primary estimand for Phase 2b.
Population-level summary: Difference in proportion of participants achieving response at Week 12 between study intervention conditions.

### Secondary Estimands for Continuous Endpoints

For secondary objectives that have continuous endpoints analyzed up to Week 12, the clinical question of interest is:
What is the difference between Compound I and placebo in the target patient population, in mean change from baseline at Week 12 if restrictions pertaining to changing permitted concomitant medications, and taking prohibited medications were not violated, and all participants adhered to the study intervention?

The estimand is described by the following attributes:
Population: Participants with moderately-to-severely active RA (see Section 5 1).
Endpoint: Change from baseline for
   DAS28-hsCRP (for Phase 2b only)
   SDAI
   CDAI
   ACR component 68 tender joint count
   ACR component 66 swollen joint count
   ACR component PhGADA_VAS
   PaGADA_VAS
   Patient's Assessment of Arthritis Pain (VAS)
   patient's assessment of physical function using HAQ-DI
   the duration and severity of morning joint stiffness
   FACIT-F scores at Week 12
   SF-36 domains
   SF-36 physical component summary, and
   SF-36 mental component summary.
ICE will be accounted using the same estimand strategy as for the primary estimand for Phase 2a.
Population-level summary: Difference in mean change from baseline to Week 12 between intervention conditions.

### Supportive Estimands

Additional details on supportive estimands for primary and secondary objectives will be provided in the SAP.

### Study Design

### Overall Design

This Study is an adaptive Phase 2a/2b multicenter, randomized, double-blind study to evaluate the efficacy and safety of multiple dose levels of Compound I in adult participants with moderately-to-severely active RA.

This table summarizes the key characteristics for the 2 phases of the study.

| Study characteristics | Phase 2a | Phase 2b |
|---|---|---|
| Type of study | Proof of concept | Dose ranging |

| | |
|---|---|
| Participant population | Participants randomly assigned to study intervention will |
| | have a history of failure^{a} to at least 1 csDMARD and either 1 |
| | bDMARD or 1 tsDMARD treatment. |
| Standard-of-care therapy | See the schema and Section 6.1.1. |

Abbreviations: bDMARD = biologic disease-modifying antirheumatic drug; csDMARD = conventional synthetic disease-modifying antirheumatic drug; tsDMARD = targeted synthetic disease-modifying antirheumatic drug.
^{a}History of failure = an inadequate response, intolerance, or loss of response.

### Study Periods

Both Phase 2a and Phase 2b will have the following study periods:
Period I: screening (Visit 1)
Period II: double-blind, placebo-controlled treatment
Period III: treatment
Period IV: post-treatment follow-up (Visits 801 and 802)

See the schema (Section 1.2) and SoA (Section 1.3) for details about the duration of the study periods.

### Screening Period

The study entry criteria will be the same for both Phase 2a and Phase 2b of this study. See Sections 5.1 and 5.2.

### Treatment Periods

This table summarizes the key features of the treatment periods for both phases of the study. For doses of Compound I, see Section 6.1. As stated in the SoA, study intervention will be self-administered by participants at home except for the day of any study visit.

| Treatment period | Phase 2a | Phase 2b |
|---|---|---|
| Period II, double-blind, | At the baseline visit (Visit 2), | At the baseline visit (Visit 2), |
| placebo-controlled treatment | participants who met the | participants who met the |
| | eligibility criteria will be | eligibility criteria will be |
| | randomly assigned, in a ratio | randomly assigned, in a ratio |
| | of 2:1, to the following study | of 2:1:2:2, to the following |
| | interventions: | planned study interventions: |
| | 50 mg QD Compound I, or | 125 mg QD Compound I |
| | placebo. | 50 mg QD Compound I |
| | | 25 mg QD Compound I, or |
| | | placebo. |

| | |
|---|---|
| Period III: treatment | At Week 14, the following participants may begin standard- |
| | of-care therapy at the investigator's discretion: |
| | all placebo participants, and |
| | Compound I participants who have not achieved low |
| | disease activity (CDAI ≤10). These participants will no |
| | longer receive the study intervention. |
| | Participants receiving Compound I who have achieved low |
| | disease activity (CDAI ≤10) will continue their assigned dose |
| | and dosing frequency. |

### Visit Structure

Some study visits will have the same numbering for both Phase 2a and 2b. The visit content may be different for different phases (see the SoA, Section 1.3). In addition, the visit intervals will differ for the 2 p60, hases.

| Phase 2a | Phase 2b |
|---|---|
| Visit 1 (screening) | |
| Visit 2 | |
| (randomization/baseline) | |
| 3 | 103 |
| 4 | 104 |
| 5 | 105 |
| 6 | 106 |
| 7 | 107 |
| 8 | 108 |
| 9 | 109 |
| 10 | 110 |
| 11 | — |
| 12 | — |
| 13 | — |
| 14 | — |

Post-treatment
follow-up:
Visit 801
Visit 802

### Joint and Safety Assessors

This study includes blinded joint and safety assessors (see Sections 6.4 and 8).

### Appropriateness of Study Population

Patients with RA are treated with the oral csDMARD methotrexate (MTX) as the first-line therapy either by itself or in combination with other therapies (Fraenkel et al. 2021; Smolen et al. 2022). If the treatment target is not achieved with the initial csDMARD strategy, treatment modification often involves use of bDMARDs, including TNF inhibitors, or targeted synthetic tsDMARDs, in combination with csDMARDs (Fraenkel et al. 2021; Smolen et al. 2022).

### Choice of Placebo Control and Number of Treatment Groups

The double-blind, placebo-controlled design of this study limits potential bias in investigator assessments and enables a clearer interpretation of the effects of active drug compared to placebo.

The use of multiple active dose levels in this study, as determined by the interim analysis from Phase 2a (Section 9.4), will allow for an evaluation of safety and efficacy across a broad dose range and so provide information to guide dose selection for future studies.

Placebo-controlled trials are justifiable when they are supported by sound methodological consideration and when the use of placebo does not expose research participants to unacceptable risk of harm. The placebo-controlled study design was selected to minimize bias for both investigators and participants. The approximately 3-month randomized, double-blind, placebo-controlled period will allow an objective assessment of the Compound I efficacy and safety in participants with RA. Given the magnitude and variation of placebo responses in RA trials, controlling for the placebo response is necessary to allow a clear interpretation of drug effect. The placebo-controlled study design utilized is supported by the FDA and EMA guidance for Developing Drugs and Biological Products for RA (FDA 2013; EMA 2017).

### Follow-Up Period Duration

The 8-week follow-up duration is considered acceptable to evaluate safety and to explore the durability of biomarker and clinical disease activity changes achieved during the treatment period.

### Primary Endpoints

Phase 2a: Change from Baseline in DAS28-hsCRP as the Primary Endpoint

The primary endpoint of this study is the change from baseline at Week 12 in the DAS28-hsCRP (Sections 3 and 8.1.12) and is a continuous measure that enables evaluation across multiple time points.

### Phase 2b: Proportion of Participants Achieving ACR50 at Week 12

The ACR50 is widely used in RA clinical trials, continues to be an accepted measure to demonstrate reduction in RA disease activity (FDA 2013), and is an appropriate primary outcome measure for an early phase dose-ranging study.

### Independent Joint and Safety Assessors

To prevent potential bias from observed efficacy or laboratory changes, a “dual assessor” approach will be used to evaluate efficacy and safety (see Section 8).

### MRI

MRI allows detailed assessment of the synovial joint. MRI features are frequently used as outcome measures in RA clinical trials. The Outcome Measures in Rheumatoid Arthritis Clinical Trials (OMERACT) RA MRI Scoring system (RAMRIS) outlines semiquantitative scoring of 5 RA pathologies: bone erosions, joint space narrowing, synovitis, tenosynovitis, and bone marrow edema in the wrist and metacarpophalangeal joints.

Both phases of this study will evaluate the effectiveness of Compound I in reducing inflammation in the joints. Enrolling study participants with active synovitis as determined by MRI will support overall evaluation of Compound I in reducing inflammation. Exploratory evaluation of a change in RAMRIS synovitis score at Week 12 will provide an objective measure of reduction in inflammation.

### Justification for Dose

The relationship between dose and efficacy for a RIPK1 inhibitor has not been established for RA. This study will evaluate the effect of multiple Compound I doses on clinical outcomes in participants with moderately-to-severely active RA.

The planned dose for Phase 2a of this study is 50 mg QD, and an additional dose up to 125 mg QD may be evaluated based on interim analyses (Section 9 4). The planned Compound I doses for Phase 2b of this study are 25, 50 and 125 mg.

The doses for Phase 2a and 2b were selected based on factors such as human safety, tolerability, and PK data from Phase 1 SAD and MAD evaluations, in vitro assays, in vivo pharmacological models in rodents, and nonclinical toxicology. In the Phase 1 clinical study, single doses of up to 1000 mg and multiple doses of 180 mg and 500 mg QD were evaluated.

A dose of 25 mg QD in humans is predicted to achieve plasma concentrations that achieve the a suitable level for RIPK1 inhibition based on in vitro assays and in vivo pharmacological models. At the highest dose of 125 mg Compound I in this study, the exposure multiple (ratio of animal to human exposure) is estimated to be 7 for AUC based on the 9-month, NOAEL of 150 mg/kg.

For Phase 2b, the number of dose levels and the dose amount are subject to change based on results from the Phase 2a interim analyses, or any other relevant data that may become available, but the highest dose in Phase 2b will not exceed 125 mg.

### End of Study Definition

The end of the study is defined as the date of the last visit of the last participant in
Phase 2a, if no Phase 2b is initiated, or
Phase 2b, if initiated.

A participant is considered to have completed the study if the participant has completed all periods of the study including the last scheduled procedure shown in the SoA.

### Study Population

Prospective approval of protocol deviations to recruitment and enrollment criteria, also known as protocol waivers or exemptions, is not permitted.

### Inclusion Criteria

Participants are eligible to be included in the study only if all of the following criteria apply:

### Age

[1] Are ≥18 years of age at the time of signing the informed consent.

### Sex and Contraceptive Requirements

[2] Are male or female.
Both women of childbearing potential (WOCBP) and women not of childbearing potential (WNOCBP) may participate in this trial.
Contraceptive use by participants should be consistent with local regulations regarding the methods of contraception for those participating in clinical studies. For the definitions and contraception requirements of this protocol, see Appendix 10 4.

### Weight

[3] Have a BMI within the range of 18 to 40 kg/m² (inclusive).

### Type of Participant and Disease Characteristics

[4] Have a diagnosis of adult onset RA for at least 3 months prior to screening, as defined by the 2010 ACR/EULAR classification criteria (Aletaha et al. 2010).

[5] Have moderately-to-severely active RA, at screening (Visit 1) and randomization (Visit 2/baseline), defined by the presence of
≥6 swollen joints based on 66 joint count, and
≥6 tender joints based on 68 joint count.

### Notes:

The distal interphalangeal joint should be evaluated but not included in the total count to determine eligibility.

If a participant has received corticosteroid treatment per Exclusion Criterion [29], the treated joint should be excluded from the joint count.

[6] Have at least 1 of the following:
positive test results for rheumatoid factor or anti-citrullinated peptide antibodies at screening,
OR
previous radiographs documenting bony erosions in hands or feet consistent with RA.

[7] Have hsCRP >1.2 times ULN per the central laboratory at screening.

[8] Have active synovitis in ≥1 joint in hands or wrists at screening as demonstrated by an MRI synovitis RAMRIS score (Østergaard et al. 2017) of ≥1 determined from central reading of images.

[9] Have up-to-date vaccination status assessment per regional or national guidelines (including ACR or EULAR guidelines; ACR 2022, Furer et al. 2020), specifically

influenza, pneumonia, SARS-COV-2, and herpes zoster (see criterion for timing of vaccinations).
Note: investigators should document the discussion with any potential participant, including decisions regarding vaccination.

Have clinical laboratory test results within normal reference range or results with acceptable deviations that are judged as not clinically significant by the investigator at screening. This table outlines laboratory test results with required ranges for inclusion in this study.

| Test | Result |
|---|---|
| Hematology | |
| ANC | ≥1.5 × 10⁹/L (≥1.5 × 10³/μL or ≥1.5 G/L) |
| Platelet count | ≥100 × 10⁹/L (≥100 × 10³/μL or ≥100 G/L) |
| Hemoglobin | ≥10.0 g/dL |
| Lymphocyte count | >800 cells/μL (>0.80 × 10³/μL or >0.80 G/L) |
| Total leukocyte count | ≥3.0 × 109/L (≥3.0 × 10³/μL or ≥3.0 G/L) |
| Clinical Chemistry | |
| Serum creatinine | Levels ≤2 × ULN |
| ALT and AST | Levels ≤ ULN |
| TBL and ALP | <1.5 × ULN (patients with Gilbert's syndrome |
| | must have serum direct bilirubin <1.5 mg/dL) |

### Prior Therapy

[11] Have had a history of failure (an inadequate response, intolerance, or loss of response) to at least 1 csDMARD, and either 1 bDMARD or 1 tsDMARD treatment. This is defined as signs and symptoms of active disease despite receiving these treatments according to local standard of care:
azathioprine
methotrexate
hydroxychloroquine
chloroquine
leflunomide
sulfasalazine
bDMARDs, or
tsDMARDs.

See Section 6 9.2 for permitted treatments during the study if the dose is stable for ≥28 days prior to the screening MRI.

Note to sites in EU Member States: See Section 10.10.1 1 for country-specific modifications to Inclusion Criterion [11].

### Informed Consent

Are capable of giving signed informed consent as described in Appendix 10.1, Section 10.1.3, which includes compliance with the requirements and restrictions listed in the ICF and in this protocol.

### Exclusion Criteria

Participants are excluded from the study if any of the following criteria apply:

### Medical Conditions

[13] Have Class IV RA according to ACR revised criteria (Hochberg et al. 1992).

[14] Have clinically significant ECG abnormalities including corrected QT interval, Fridericia's correction >450 msec for males and >470 msec for females.

[15] Have a history of additional risk factors for Torsades de Pointes such as, heart failure, hypokalemia, or a family history of long QT syndrome.

[16] Have clinically relevant abnormal blood pressure or heart rate as determined by the investigator.

[17] Have presence of 1 or more significant concurrent medical conditions per investigator judgment, including but not limited to
poorly controlled diabetes or hypertension
chronic kidney disease stage III a or b, IV, or V
symptomatic heart failure according to New York Heart Association class II, III, or IV
myocardial infarction, unstable angina pectoris, stroke, or transient ischemic attack within the past 12 months before randomization
severe chronic pulmonary disease, for example, requiring oxygen therapy
major chronic inflammatory disease or connective tissue disease other than RA, including but not limited to
   systemic lupus erythematosus
   psoriatic arthritis
   axial spondyloarthritis, including ankylosing spondylitis and non-radiographic axial spondyloarthritis
   reactive arthritis
   gout
   scleroderma
   polymyositis
   dermatomyositis
   active fibromyalgia, or
   multiple sclerosis.

[18] Have a history of chronic alcohol abuse, IV drug abuse, cannabis use disorder, or illicit drug abuse within 1 year before screening.
Note: Cannabis use is prohibited during participation in this study, regardless of local laws or if used for medical purposes (Section 6.9.3).
CBD products may be used during the study if they are derived exclusively from hemp. Participants who use hemp-based CBD products must be on a stable dose for at least 10 days prior to randomization, and participants must remain on that stable dose during the study (Section 6.9.2).

[19] Have a C-SSRS ideation within 1 month prior to screening or any suicidal behavior within 3 months prior to screening and either ideation or suicidal behavior during screening prior to randomization.

[20] Have a diagnosis or history of malignant disease within 5 years prior to baseline, with the exceptions of:
basal cell or squamous epithelial carcinomas of the skin that have been resected with no evidence of metastatic disease for 3 years, or
cervical carcinoma in situ, with no evidence of recurrence within the 5 years prior to baseline

[21] Have eGFR of <60 mL/minute from serum creatinine using the CKD-EPI creatinine equation (2021).

[22] Have had any surgical procedure within 12 weeks prior to screening, or any planned surgical procedure scheduled to occur during the study, with the exception of minor surgery requiring local or no anesthesia and without any complications or sequelae.

[23] Have had any of the following types of infection within 3 months of screening or develops any of these infections before the randomization visit:
Serious (requiring hospitalization, and/or IV or equivalent oral antibiotic treatment)
Opportunistic (as defined in Appendix 10.8)
Chronic (duration of symptoms, signs, and/or treatment of 6 weeks or longer)
Recurring (including, but not limited to herpes simplex, herpes zoster, recurring cellulitis, chronic osteomyelitis)
   Note: Herpes zoster is considered active and ongoing until all vesicles are dry and crusted over.
   Exception: If the investigator determines that a participant with recurrent nonserious infections such as cellulitis and uncomplicated orolabial or genital herpes, is not at an increased risk of complications.

[24] Have any of these infections
HIV infection.
current infection with HBV, that is, positive for HBsAg and/or PCR positive for HBV DNA.
current infection with HCV, that is, positive for HCV RNA, or
active TB.

[25] Have or have had LTBI that has not been treated with a complete course of appropriate therapy as defined by the WHO or the United States CDC, unless such treatment is underway, as per Section 8.2 9.

[26] Have a current or recent acute active infection, or fever of 100.5° F. (38° C.) or above, at screening or baseline. For at least 30 days prior to screening, participants must have no symptoms and/or signs of confirmed or suspected infection, and must have completed any appropriate anti-infective treatment.
Note: Participants who have an upper respiratory infection, a vaginal candida infection, or an oral candida infection and who are being treated only symptomatically and not requiring systemic anti-infectives may be considered for enrollment if other study eligibility criteria are met. Enrollment of participants with other uncomplicated local infections should be discussed with the sponsor's designated medical monitor.

[27] Are women who are currently pregnant or breastfeeding, or who intend to become pregnant or to breastfeed at any time during the study or within at least 28 days after receiving the last dose of study intervention.

### Prior/Concomitant Therapy

[28] Have failed more than 3 advanced therapies, which includes bDMARDs and tsDMARDs.

[29] Are currently receiving or have received any of these therapies within 28 days prior to the screening MRI.

| Therapy | Notes |
|---|---|
| Unstable dose defined as any prescription | Includes a planned dose change during the |
| change or change in dose for | study, including initiation or discontinuation |
| methotrexate | of treatment. |
| hydroxychloroquine | |
| chloroquine | |
| sulfasalazine, or | |
| leflunomide. | |
| Oral Janus kinase inhibitor | Examples include tofacitinib or baricitinib. |
| Parenteral corticosteroids | Includes initiation of treatment during the |
| | study. |
| | Note: |
| | A single intra-articular corticosteroid |
| | injection of ≤40 mg triamcinolone, or |
| | equivalent is permitted within 28 days prior to |
| | the screening MRI if no additional injections |
| | are planned during the study. |
| | The treated joint should be excluded from any |
| | joint-specific evaluations during the study. |
| Chronic opioid drug at an unstable dose. | Includes planned dose increases or a new |
| | prescription during the study. |
| Cyclophosphamide | |
| Azathioprine | |
| Cyclosporine | |
| Gold | |
| Mycophenolate mofetil | |
| Prosorba ® column apheresis, or | |
| Tacrolimus. | |

[30] Have received these treatments prior to screening MRI or plan on receiving any of these biologic immunosuppressive treatments during the study

| Treatment within | |
|---|---|
| 8 weeks prior to | Treatment within 12 months prior to |
| screening MRI | screening MRI |
| etanercept | B-cell-depleting agents, such as rituximab |
| adalimumab | other cell-depleting biologics, such as anti- |
| infliximab | CD3 antibody |
| certolizumab pegol | |
| golimumab | |
| anakinra | |
| abatacept | |
| tocilizumab | |
| sarilumab | |

Note:
Other biologic agents may be allowed after discussion with the sponsor.

[31] Did not have a primary response (that is, response within first 12 weeks of treatment) to treatment with most recent TNF-α antagonist, per investigator assessment.

[32] Are using medications, supplements, or dietary substances that are strong CYP3A4 inhibitors or inducers within 14 days prior to baseline or plan to use these medications during the study (see Section 6.9.3).

[33] Plan to receive treatment with medications that are sensitive CYP3A substrates or P-gp substrates with a narrow therapeutic index (see Section 6.9 3).

[34] Have received live vaccine(s), including live attenuated vaccines, within 4 weeks prior to screening or intend to receive during the study and is within 5 half-lives after the last dose of intervention.

### Exceptions:

Non-live or inactivated vaccine if received at least 2 weeks before baseline or after the last study visit
Inactivated influenza and pneumococcal vaccines, SARS-COV-2 vaccines and non-live herpes zoster vaccines authorized by local regulatory bodies can be given at any time during the study.

[35] Have received a BCG vaccination or treatment within less than 4 weeks before randomization, or intend to receive BCG vaccination or treatment during the study, or within at least 5 half-lives after receiving the last dose of study intervention.

### Prior/Concurrent Clinical Study Experience

[36] Were previously enrolled in a clinical study investigating RIPK1 (Compound I), including Phase 2a of this study, or any other molecule targeting RIPK1 for the treatment of autoimmune or auto-inflammatory conditions.

[37] Have participated, within the last 30 days, in a clinical trial involving an investigational study intervention. If the previous study intervention has a long half-life, 3 months or 5 half-lives (whichever is longer) should have passed before screening.

[38] Have previously completed or withdrawn from this study.

[39] Are currently enrolled in any other clinical trial involving an investigational product or any other type of medical research judged not to be scientifically or medically compatible with this study.

### Other Exclusion Criteria

Have contraindications to MRI; for example
claustrophobia, pacemakers, aneurysm clips, and intraocular metallic fragments; or
IV gadolinium-based contrast agent, including but not limited to moderate or severe renal insufficiency or prior allergic reaction to gadolinium-containing contrast media.

[41] Have experienced hypersensitivity to the active substance or to any of the excipients of Compound I.

[42] Have donated more than a single unit of blood within 4 weeks before randomization or intent to donate blood during the study.

[43] Are investigator site personnel directly affiliated with this study and/or their immediate families. Immediate family is defined as a spouse, parent, child, or sibling, whether biological or legally adopted.

[44] Are Lilly employees or employees of third-party organizations involved with the study that require exclusion of their employees.

[45] Are unsuitable for inclusion in the study, in the opinion of the investigator or sponsor, for any reason that may compromise the participant's safety or confound data interpretation.

### Study Intervention(s) and Concomitant Therapy

Study intervention is defined as any investigational intervention(s), marketed product(s), placebo, or medical device(s) intended to be administered to/used by a study participant according to the study protocol.

### Study Intervention(s) Administered

As stated in the SoA, study intervention will be self-administered by participants at home except for the day of any study visit. On visit days the participant can take the study inventions after blood samples are drawn as specified in the SoA.

These tables list the interventions used in different phases of this clinical study.

Phase 2a of This Study

| Intervention Name | Compound I^{a} | Placebo |
|---|---|---|
| Dose Level | 50 mg | Not |
| | | applicable |
| Frequency of Administration | QD | QD |
| Route of Administration | Oral | Oral |
| Authorized as Defined by EU | Not authorized | Not |
| Clinical Trial Regulation | | authorized |

For standard-of-care therapy used during Phase 2a, see Section 6.1.1.
^{a}A higher dose level cohort, not to exceed a maximum of 125 mg, may be added during Phase 2a based on the results of the interim analyses and recommendations of the sponsor's IAC (Section 9.4).

Phase 2b of This Study

| Intervention Name | Compound I^{a} | Placebo |
|---|---|---|
| Planned Dose Levels | 125 mg | Not |
| | 50 mg | applicable |
| | 25 mg | |
| Frequency of Administration | QD | QD |
| Route of Administration | Oral | Oral |
| Authorized as Defined by EU | Not authorized | Not |
| Clinical Trial Regulation | | authorized |

For standard-of-care therapy used during Phase 2b, see Section 6.1.1.
^{a}The final doses of Compound I in Phase 2b will be determined based on the interim analyses from Phase 2a of this study and recommendations of the sponsor's IAC (Section 9.4).

### Packaging and Labeling

Study interventions will be supplied by the sponsor or its designee in accordance with current Good Manufacturing Practice. Study interventions will be labeled as appropriate for country requirements.

### Standard-of-Care Therapy

All placebo participants may begin SOC therapy at Week 14 at the investigator's discretion. The SOC therapy includes all the csDMARDs and b/tsDMARDs.

Participants who were randomly assigned to Compound I at baseline and did not achieve LDA (CDAI≤10) at Week 14 will no longer receive the study intervention and may receive SOC therapy at the investigator's discretion.

Name(s) and dosage regimen(s) must be recorded for the SOC therapy.

### Intra-Articular Joint Injections and Bursal Injections

These may be given at doses and intervals at the investigator's discretion. If a participant receives intra-articular joint injections, the treated joints should be recorded as “not evaluable” on subsequent joint-specific assessments for the remainder of the study.

### Assignment to Study Intervention

Assignment to treatment groups within this protocol will be determined by a computer-generated random sequence using an IWRS. For randomization ratios, see Section 4.1.

Participants will be stratified at baseline by
geographic region:
   North America
   Latin America
   Europe, and
   rest of the world.
history of failure (intolerance to medication, inadequate response, or loss of response) to bDMARDs or tsDMARDs, total number (1, >1), and
SJC66 increase from screening to baseline ≥20% versus <20%.

### Blinding

Some of the treatment periods for this study are double-blind. Blinding will be maintained throughout the conduct of the study, as described in the separate Blinding and Unblinding Plan.

### Blinded Joint and Safety Assessors

To minimize bias due to observed efficacy or laboratory changes, a “dual assessor” approach will be used to evaluate efficacy and safety. See Section 8.

### Emergency Unblinding

The IWRS will be programmed with blind-breaking instructions. In case of an emergency, the investigator has the sole responsibility for determining if unblinding of a participant's intervention assignment is warranted. Participant safety must always be the first consideration in making such a determination.

If a participant's intervention assignment is unblinded, the sponsor must be notified immediately within 24 hours of this occurrence. The date and reason that the blind was broken must be recorded.

Discontinuation from the Study Intervention in Case of Unblinding

If an investigator, site personnel performing assessments, or participant is unblinded, the participant must be discontinued from the study intervention (Section 7.1.2).

### Study Intervention Compliance

Participant compliance with study intervention will be assessed at each visit by counting returned tablets.

A participant will be considered significantly noncompliant if they miss more than 20% of the prescribed doses of study intervention during the study, unless the participant's study intervention is withheld by the investigator for safety reasons. Similarly, a participant will be considered significantly noncompliant if they are judged by the investigator to have taken 20% more than the prescribed amount of medication during the study.

Participants will be counseled by study staff on the importance of taking the study intervention as prescribed, as appropriate.

A record of the number of tablets dispensed to and taken by each participant must be maintained and reconciled with study intervention and compliance records. Intervention start and stop dates, including dates for intervention interruptions will also be recorded in the CRF.

### Dose Modification

This protocol does not allow dose adjustments.

Continued Access to Study Intervention after the End of the Study

Study intervention will not be available to participants after completion of the study unless required per local regulations.

### Prior and Concomitant Therapy

### General Considerations

Participants should consult the investigator or other appropriate study personnel at the site before taking any new medications or supplements during the study.

Avoid prescribing additional medications during the study unless required to treat an AE or for the treatment of an ongoing medical condition. Investigators should follow local guidelines for the management of lipid disorders.

As stated in Section 7.1.2, if the need for other concomitant medications arises, discontinuation of the participant from study intervention or the study will be at the discretion of the investigator in consultation with sponsor or designee.

Clinical drug-drug interaction studies have not yet been conducted with Compound I. However, based upon in vitro studies, Compound I may inhibit drugs that are metabolized by CYP2C8, CYP2C9, CYP2D6, and CYP3A, and may induce CYP3A5. Compound I may also inhibit drugs that are substrates of P-gp, BCRP, OATP1B3, OCT1, and OATP2B1 transporters (for additional information, see the IB).

Information regarding prohibited concomitant medications due to possible drug-drug interaction is provided in Section 6.9.3.

Guidance if prescribed the medications below:

Investigators should cautiously consider concomitant use of the following BCRP-sensitive substrates while participants are receiving study intervention: coumestrol, daidzein, genistein, prazosin, and rosuvastatin.

### Concomitant Therapy Data Collection

Any medication or vaccine, including over-the-counter or prescription medicines, vitamins, and/or herbal supplements, that the participant is receiving at the time of enrollment or receives during the study must be recorded along with
name of medication, vaccine, or therapy
reason for use
route of administration, and
dates of administration including start and end dates.

Study personnel should collect additional dosing information, including dose and frequency, for the following:
Phase 2a and Phase 2b: permitted RA therapies

The medical monitor should be contacted if there are any questions regarding concomitant or prior therapy.

### Permitted Concomitant Medications

Unless specified otherwise, this table outlines the allowed concomitant therapies during the study if the dose is stable for ≥28 days prior to the screening MRI. These permitted concomitant medications should remain stable unless specified otherwise in the table.

| Permitted concomitant therapy | Comments |
|---|---|
| Methotrexate (MTX) | Allowed doses (should be according to the local |
| | standard of care if local doses are standard) |
| | parenteral MTX up to 20 mg/week OR |
| | oral MTX up to 25 mg/week. |
| | Participants on MTX should receive a folic acid |
| | supplement according to local standard of care. |
| Hydroxychloroquine | Doses up to 400 mg daily. |
| Chloroquine | Doses up to 250 mg daily. |
| Leflunomide | Doses up to 20 mg daily. |
| Oral sulfasalazine | Doses up to 3000 mg daily. |
| Corticosteroids | Oral prednisone doses of up to 12.5 mg daily or |
| | other equivalent corticosteroid dose. |
| | Topical, intranasal, intraocular, and inhaled |
| | corticosteroids are permitted. |
| Nonsteroidal anti-inflammatory drugs | The participant must be on a stable dose for at |
| (NSAIDs) | least 7 days before planned randomization. |
| | Increases in dose or introduction of new NSAIDs |
| | are not allowed during the study. |
| Analgesics | Dose reductions or termination of analgesics are |
| | allowed at any time. |
| Folic acid | Local standard of care should be followed for |
| | concomitant administration of folic acid. |
| Cannabidiol (CBD) products, if derived | Participants who use hemp-based CBD products |
| exclusively from hemp | must be on a stable dose for at least 10 days prior |
| | to randomization, and participants must remain on |
| | that stable dose during the study. |
| Vaccinations | Prior use: see Inclusion Criterion [9] and |
| | exceptions for Exclusion Criterion [34]. |
| | During the study: Non-live seasonal vaccinations |
| | and/or emergency vaccinations, such as rabies or |
| | tetanus vaccinations, are allowed. |

### Prohibited Concomitant Medications

This table outlines the prohibited concomitant medications during the study. Unless specified otherwise, this table applies to both Phase 2a and Phase 2b of this study.

If a prohibited treatment listed here is required, the study intervention should be permanently discontinued (Section 7.1.2).

| Prohibited concomitant therapy | Comments |
|---|---|
| RA-specific treatments | See Section 5.2, Exclusion Criteria |
| | [29] and [30]. |
| | Systemic corticosteroids (other than |
| | those permitted in this study; Section |
| | 6.9.2), including intra-muscular or |
| | intra-articular corticosteroids, are not |
| | allowed during the study. |
| Other concomitant therapy and vaccinations | |
| Due to DDI risks, concomitant medications or herbal | Prohibited within 14 days prior to |
| supplements prohibited in this study include, but are | baseline and during the study. |
| not limited to the following drugs: | |
| Strong CYP3A4 inhibitors | |
| Atazanavir, boceprevir, ceritinib, | |
| clarithromycin, cobicistat, conivaptan, | |
| idelalisib, itraconazole, josamycin, | |
| ketoconazole, lonafarnib, mifepristone, | |
| nefazodone, nelfinavir, posaconazole, | |
| ribociclib, telithromycin, tucatinib, | |
| voriconazole | |
| Ritonavir (by itself, or in combination with | |
| danoprevir, elvitegravir, fosamprenavir, | |
| indinavir, lopinavir, paritaprevir, saquinavir, | |
| tipranavir) | |
| Strong CYP3A4 inducers | |
| Aminoglutethimide, apalutamide, | |
| carbamazepine, enzalutamide, fosphenytoin, | |
| ivosidenib, lumacaftor, mitotane, | |
| phenobarbital, phenytoin, rifabutin, rifampin | |
| (rifampicin), rifapentine, St. John's Wort | |
| Sensitive CYP3A4 substrates with narrow | |
| therapeutic index | |
| Alfentanil, atorvastatin, lovastatin, | |
| midazolam, quetiapine, quinidine, sildenafil, | |
| simvastatin, sirolimus, tacrolimus, triazolam | |
| Transporter P-gp substrates with narrow | |
| therapeutic index | |
| Colchicine, cyclosporine, dabigatran | |
| etexilate, digoxin, everolimus, pimozide, | |
| quinine | |
| Cannabis | Cannabis use is prohibited during |
| | participation in this study, regardless |
| | of local laws or if used for medical |
| | purposes. |
| Vaccinations | Prior use: see Exclusion Criterion |
| | [34]. |
| | During the study: Live vaccinations, |
| | including live herpes zoster |
| | vaccination, are not allowed any time |
| | during the study. |

Abbreviation: DDI = drug-drug interaction.

### Discontinuation of Study Intervention and Participant Discontinuation/Withdrawal

This section describes reasons for a participant's
temporary or permanent discontinuation of study intervention (Section 7.1), or
discontinuation (withdrawal) from the study (Section 7.2).

Discontinuation of specific sites or of the study as a whole is handled as part of Appendix 10.1. In addition, Appendix 10.1 lists the criteria related to pausing of enrollment to the study.

Unless specified otherwise, the discontinuation criteria apply both to Phase 2a and Phase 2b of this study.

### Discontinuation of Study Intervention

### Temporary Discontinuation of Study Intervention

### Infection-Related Criteria for Temporary Discontinuation of Study Intervention

| If a participant develops . . . | Then follow this guidance . . . |
|---|---|
| serious or opportunistic infections, as defined | Withhold study intervention until resolution |
| in the exclusion criteria (Section 5.2) | of all acute clinical signs and symptoms, and |
| | completion of all appropriate anti-infective |
| | treatment (except for LTBI, noted below). |
| | The investigator should consult with the |
| | sponsor-designated medical monitor to |
| | determine when it is appropriate to restart |
| | study intervention. |
| Any acute infection or illness | At the discretion of the investigator and |
| | sponsor or its designee, withhold intervention |
| | until resolution of all acute clinical signs and |
| | symptoms, and completion of all appropriate |
| | anti-infective treatment. |
| Positive HBV DNA results | Contact the sponsor-designated medical |
| or | monitor. |
| results detecting HBV DNA, but the HBV | Repeat HBV DNA testing as soon as |
| DNA is below the level of quantification | possible. |
| | If HBV DNA is confirmed as positive, |
| | then permanently discontinue |
| | intervention (Section 7.1.2). |
| LTBI and the participant is a candidate for | Withhold study intervention for at least |
| LTBI treatment | the first 4 weeks of LTBI treatment. |
| | If there is no evidence of hepatotoxicity |
| | (ALT/AST must remain ≤2 times ULN) |
| | or other treatment intolerance after |
| | receiving at least 4 weeks of appropriate |
| | LTBI therapy, per WHO or CDC |
| | guidelines, then restart study intervention. |
| | The participant must complete |
| | appropriate LTBI therapy to remain |
| | eligible to receive study intervention. |

### Liver Test Abnormality

See Section 7.1.3.

### QTc Stopping Criteria

See Section 7.1.4.

### Abnormal Hematology Laboratory Values

See Section 7.1.5.

### Permanent Discontinuation of Study Intervention

When necessary, a participant may be permanently discontinued from study intervention. If so, the participant will discontinue the study intervention (treatment) and should remain in the study and follow procedures for all remaining study visits, as shown in the SoA.

Possible reasons for permanent discontinuation of study intervention include, but are not limited to, the following:
Participant decision: The participant asks to stop receiving study intervention.
Investigator opinion: In the opinion of the investigator, the participant should permanently discontinue the study intervention for safety reasons.
Pregnancy: The participant becomes pregnant (Sections 8.2.6 and 8.3.2).
Malignancy: The participant develops a malignancy (except for successfully treated basal or squamous cell skin carcinoma).
HIV: The participant develops HIV infection.
Active TB or untreated LTBI: The participant develops active TB or has untreated LTBI (Section 8.2.9).
HBV or HCV: The participant tests positive for HBV DNA or HCV RNA (see Sections 8.2 10 and 8.2.11 for details).
   Note: Prior to discontinuation of any immunomodulatory and/or immunosuppressive therapy, including study intervention, the participant is to be referred to, evaluated, and managed by a specialist physician with expertise in evaluation and management of viral hepatitis. The timing of discontinuation from study intervention relative to the initiation of any antiviral treatment for hepatitis is to be based on the recommendation of the consulting specialist physician, in conjunction with the investigator, and aligned with medical guidelines and standard of care.

### Suicidal Ideation and Behavior: The Participant

answered “yes” to Question 4 or Question 5 on the “Suicidal Ideation” portion of the C-SSRS, or
answered “yes” to any of the suicide-related behaviors on the Suicidal Behavior portion of the C-SSRS.

A psychiatrist or appropriately trained professional may assist in the decision to discontinue the participant.
Liver test abnormality: Section 7.1.3 describes temporary interruption of study drug based on liver test abnormalities. If, after temporary interruption of study drug, liver test results fail to return to baseline and a self-limited non-drug etiology is not identified, study drug is to be permanently discontinued.
QTc stopping criteria: See Section 7 1.4.
Abnormal hematology laboratory values: See Section 7.1.5.
Use of prohibited concomitant medications: The participant requires treatment with prohibited medications specified in Section 6.9.3. The permanent discontinuation from the study intervention should occur before introduction of a prohibited medication.
Noncompliance: The investigator decides the participant is noncompliant with study drug administration or any other study procedure.
Unblinding: If an investigator, blinded site personnel, blinded designees who are performing assessments, or the participant is unblinded to the participant's intervention assignment because of an emergency unblinding as described in Section 6.4, the participant must be permanently discontinued from study drug. In cases where there are ethical reasons for the participant to continue in the study and continue to receive study drug, the investigator must obtain specific approval from the sponsor's designated medical monitor for the participant to continue.

### Liver Chemistry Stopping Criteria

### Interrupting Study Drug Based on Elevated Liver Tests

The study drug should be interrupted and close hepatic monitoring initiated (see Section 8.2.8) if 1 or more of these conditions occur:

| Elevation | Exception |
|---|---|
| ALT or AST >8× ULN | |
| ALT or AST >5× ULN for more than 2 weeks | |
| ALT or AST >3× ULN and either TBL >2× | For participants with Gilbert's syndrome: |
| ULN or INR >1.5 | If baseline direct bilirubin is >0.5 mg/dL, then |
| | doubling of direct bilirubin should be used for |
| | drug interruption decisions rather than TBL |
| | >2× ULN. |
| ALT or AST >3× ULN with the appearance of | |
| fatigue, nausea, vomiting, right upper quadrant | |
| pain or tenderness, fever, rash, and/or | |
| eosinophilia (>5%) | |
| ALP >3× ULN, when the source of increased | |
| ALP is the liver | |
| ALP >2.5× ULN and TBL >2× ULN | For participants with Gilbert's syndrome: |
| | If baseline direct bilirubin is >0.5 mg/dL, then |
| | doubling of direct bilirubin should be used for |
| | drug interruption decisions rather than TBL |
| | >2× ULN. |
| ALP >2.5× ULN with the appearance of | |
| fatigue, nausea, vomiting, right upper quadrant | |
| pain or tenderness, fever, rash, and/or | |
| eosinophilia (>5%) | |

Source: FDA 2009 and other consensus guidelines, with minor modifications

Interrupting Study Drug Based on Elevated Liver Tests in Participants with Abnormal Baseline Liver Tests

In study participants with abnormal baseline liver tests (ALT, AST, ALP ≥1.5×ULN), the study drug should be interrupted if 1 or more of these conditions occur:

| Elevation | Exception |
|---|---|
| ALT or AST >4× baseline | |
| ALT or AST >3× baseline for more than 2 weeks | |
| ALT or AST >2× baseline and either TBL >2× ULN | For participants with Gilbert's |
| or INR >1.5 | syndrome: |
| | If baseline direct bilirubin is >0.5 |
| | mg/Dl, then doubling of direct |
| | bilirubin should be used for drug |
| | interruption decisions rather than |
| | TBL >2× ULN. |
| ALT or AST >2× baseline with the appearance of | |
| fatigue, nausea, vomiting, right upper quadrant pain | |
| or tenderness, fever, rash, and/or eosinophilia (>5%) | |
| ALP >2.5× baseline, when the source of increased | |
| ALP is the liver | |
| ALP >2× baseline and TBL >2x ULN | For participants with Gilbert's |
| | syndrome: |
| | If baseline direct bilirubin is >0.5 |
| | mg/Dl, then doubling of direct |
| | bilirubin should be used for drug |
| | interruption decisions rather than |
| | TBL >2× ULN. |
| ALP >2× baseline with the appearance of fatigue, | |
| nausea, vomiting, right upper quadrant pain or | |
| tenderness, fever, rash, and/or eosinophilia (>5%) | |

Source: FDA 2009 and other consensus guidelines, with minor modifications

Resuming or Permanently Discontinuing Study Drug after Elevated Liver Tests

Resumption of the study drug can be considered only in consultation with the Lilly-designated medical monitor and only if the liver test results return to baseline and if a self-limited non-study-drug etiology is identified. Otherwise, the study drug should be discontinued.

### QTc Stopping Criteria

If a clinically significant finding is identified (including, but not limited to changes from baseline in QT interval corrected using Fridericia's formula [QTcF]) after enrollment, the investigator or qualified designee will determine if the participant can continue on the study intervention and if any change in participant management is needed. This review of the ECG printed at the time of collection must be documented. Any new clinically relevant finding should be reported as an AE.

### Hematology Laboratory Criteria

The study intervention should be interrupted or discontinued if 1 or more of these conditions occur:

| | Resume Study Intervention after Approval |
|---|---|
| Hold Study Intervention if the Following | from Sponsor (or its Designee) if the |
| Laboratory Test Results Occur: | Following Laboratory Results Occur: |
| WBC count <2000 cells/μL | WBC count ≥2500 cells/μL |
| ANC <1000 cells/μL | ANC >1500 cells/μL |
| Lymphocyte count <500 cells/μL | Lymphocyte count ≥750 cells/μL |
| Platelet count <75,000/μL | Platelet count ≥100,000/μL |
| Hemoglobin <8 g/dL | Hemoglobin ≥10 g/dL |

Abbreviations: ANC = absolute neutrophil count; WBC = white blood cell.

### Study Assessments and Procedures

Study procedures and their timing are summarized in the SoA.

Immediate safety concerns should be discussed with the sponsor immediately upon occurrence or awareness to determine if the participant should continue or discontinue study intervention.

Adherence to the study design requirements, including those specified in the SoA, is essential and required for study conduct.

All screening evaluations must be completed and reviewed to confirm that potential participants meet all eligibility criteria. The investigator will maintain a screening log to record details of all participants screened and to confirm eligibility or record reasons for screening failure, as applicable.

### Joint and Safety Assessors

As stated in Section 6.4, to prevent potential bias from observed efficacy or laboratory changes, a “dual assessor” approach will be used to evaluate efficacy and safety.

The same assessor should perform the joint assessments of a participant, whenever possible, throughout the study to minimize interobserver variation.

A back-up independent Joint Assessor should be identified.

It is the responsibility of the principal investigator to ensure that all assessors are qualified according to the protocol specifications and trained to perform joint assessments and that all training is documented. If the independent assessor is not available, the pre-identified back-up assessor should perform such assessments.

If the principal investigator takes the role of Joint Assessor, they may not access or discuss with the participant the patient-reported assessments, PhGADA_VAS, and safety assessments. These assessments must be delegated to a qualified sub-investigator and documented on the study delegation log. The principal investigator still has primary responsibility for oversight of all participant safety throughout the trial.

### Joint Assessor

### Qualifications

The Joint Assessor, or designee, should be a rheumatologist, nurse, physician's assistant, or physician, and a skilled Joint Assessor. Other qualified personnel could be considered but must first receive sponsor approval.

### Responsibilities

The Joint Assessor is responsible for completing the joint counts.

The Joint Assessor must not access or discuss with the participant the patient-reported assessments, PhGADA_VAS, and safety assessments.

### Access to Study Forms

Site personnel assigned to the Joint Assessor role will have access to complete the Joint Evaluation and the Joint Evaluation Attestation forms.

### Scribe

Site personnel assigned to the study coordinator role will have access to complete the Joint Evaluation form (acting as a scribe). If a scribe is utilized, the Joint Assessor must still perform the joint evaluation on the participant, must review the form entries, and must complete the Joint Evaluation Attestation.

### Safety Assessor

### Qualifications

The Safety Assessor, or designee, should be a medical professional, defined as
medical doctor (MD)
doctor of osteopathic medicine (DO)
physician's assistant
nurse practitioner (NP), or
registered nurse (RN).

Other qualified individuals may be considered based upon regionally defined medical roles in consultation with the sponsor.

### Responsibilities

The Safety Assessor will have access to both safety and efficacy data and will be responsible for completing the PhGADA_VAS.

The Safety Assessor will have access to source documents, laboratory results, and CRFs, and will be responsible for making treatment decisions based on a participant's clinical response and laboratory parameters.

### Efficacy Assessments

See Section 3 for distinction between
the primary, secondary, and exploratory measures of efficacy and quality of life, and
endpoints specific to Phase 2a or Phase 2b.

Unless specified otherwise, assessments listed in this section apply to both Phase 2a and Phase 2b of this study.

### Order of Assessments

The patient- and clinician-reported assessments should be completed in the order specified in the SoA.

### Additional Calculations and Assessments

In addition to the specific assessments listed in the SoA, efficacy measurements also include calculations and assessments by
ACR response
DAS28-hsCRP
DAS28-ESR
SDAI
CDAI, and
MRI.

### Patient's Global Assessment of Arthritis Pain VAS

The Patient's Global Assessment of Arthritis Pain VAS is a single-item, patient-reported outcome instrument used to assess the current severity of patient's pain in relation to their rheumatoid arthritis.

The Pain VAS is a continuous scale comprised of a horizontal or vertical line 0- to 100 mm in length, anchored by 2 verbal descriptors at the ends
0=no pain, and
100=worst possible pain.

The participant is asked to place a line perpendicular to the VAS line at the point that represents their pain intensity. Higher scores represent greater pain intensity.

### The Pain VAS

takes less than 1 minute to complete
is used as a component of ACR, and
should be administered before any clinical assessments.

### Health Assessment Questionnaire-Disability Index (HAQ-DI)

The HAQ-DI is a patient-reported outcomes questionnaire that is commonly used in rheumatoid arthritis to measure disease-associated disability (assessment of physical function). It consists of 20 questions referring to 8 domains of functioning:
dressing/grooming—2 items
eating—3 items
hygiene—3 items
grip—3 items
arising—2 items
walking—2 items
reach—2 items, and
daily activities—3 items.

Participants assess their degree of difficulty when performing the above activities over the past week on a 4-item ordinal scale ranging from 0, without any difficulty, to 3, unable to do. In addition, there are 4 questions asking the participants if assistance from another person, aids, or devices are usually required for any of the activities mentioned above (Fries et al. 1980, 1982; Fries 1983; Ramey et al. 1996).

For each domain, the score given to that domain is the worst score within the domain, for example, if the score for one question is 1 and another 2, then the score for the domain is 2.

In addition, if an aide or device is used or if help is required from another individual, then the minimum score for that domain is 2. If the domain score is already 2 or more, then no modification is made.

The 8 scores of the 8 domains are summed and divided by 8. The result is the disability index or functional disability index. Higher scores indicate more limitations in physical function.

The HAQ-DI can be completed in approximately 5 minutes.

The validity, reliability, and sensitivity to change of the HAQ-DI have been established in numerous observational and clinical studies (Fries et al. 1980, 1982; Wells et al. 1993; Bruce and Fries 2003).

The HAQ-DI should be administered before any clinical assessments.

### Patient's Global Assessment of Disease Activity (RA) (PaGADA_VAS)

The PaGADA_VAS is a single-item, patient-reported outcome instrument that asks the participant how they feel their RA is today.

The PaGADA_VAS is a continuous scale comprised of a horizontal or vertical line 0 to 100 mm in length, anchored by 2 verbal descriptors at the ends:
0=very well, and
100=very poor.

The participant is asked to place a line perpendicular to the VAS line at the point that represents their RA activity. Higher scores represent a higher level of disease activity (Nikiphorou et al. 2016).

### The PaGADA_VAS

is used as a component of ACR, and
should be administered before any clinical assessments.

### Short Form-36-Item Health Survey Acute Form (SF-36v2 Acute)

The SF-36v2 acute is a subjective, generic, health-related quality of life instrument that is patient-reported and consists of 36 questions covering 8 health domains:
physical functioning
bodily pain
role limitations due to physical problems
role limitations due to emotional problems
general health perceptions
mental health
social function, and
vitality.

The SF-36 can be scored into the 8 health domains named above and 2 overall summary scores, the physical component summary and the mental component summary (McHorney et al. 1993; Ware et al. 2007).

The SF-36 version 2 acute version uses the recall period of “the past week” (Ware and Sherbourne 1992; Maruish 2011).

The participant's responses for each domain are asked using Likert scales that vary in length, with 3 to 6 response options per item. Each domain is scored by summing the individual items and transforming the scores into a 0 to 100 scale, with higher scores indicating better health status or functioning. The 2 summary scores are based on the scores from the 8 SF-36v2 Acute domains.

### Morning Joint Stiffness Duration PRO

The Duration of Joint Stiffness PRO is a single-item, participant-administered scale designed to capture information on the self-reported length of time that a participant's joint stiffness lasted each day.

Participants report the duration of time by entering the number of hours and minutes their joint stiffness lasted today.

### Morning Joint Stiffness Severity NRS

The Joint Stiffness Severity NRS is a single-item, participant-administered, 11-point horizontal scale that captures the severity of morning joint stiffness using a scale from 0 to 10, where
0=no joint stiffness, and
10=joint stiffness as bad as you can imagine.

Overall severity of a participant's joint stiffness is indicated by selecting the number that best describes the worst level of joint stiffness on the day of visit.

### Functional Assessment of Chronic Illness Therapy-Fatigue (FACIT-F)

The FACIT-F scale (Cella 1997) is a 13-item questionnaire that measures severity of fatigue and its impact upon daily activities and functioning over the past 7 days.

The FACIT-F uses a 5-point Likert scale for scoring. Total scores range from 0 to 52, with higher scores indicating less fatigue.

### Clinician-Administered Tender/Swollen Joint Count (TSJC) (68/66)

The clinician-administered TSJC (68/66) will be performed at visits according to the SoA.

The same assessor should perform the joint assessments of a participant, whenever possible, throughout the study to minimize interobserver variation. See Section 8 for descriptions of the Joint Assessor, Safety Assessor, and scribe.

### Tender Joint Counts

The number of tender joints will be determined by examination of 68 joints, 34 joints on each side of the participant's body.

The 68 joints to be assessed and classified as tender or not tender include

| | |
|---|---|
| 2 temporomandibular joints | 2 sternoclavicular joints |
| 2 acromioclavicular joints | 2 shoulder joints |
| 2 elbow joints | 2 wrist joints |
| 2 hip joints | 2 knee joints |
| 2 ankle joints | 2 tarsal joints, and |
| hands | feet |
| 10 metacarpophalangeal joints | 10 metatarsophalangeal joints |
| 2 interphalangeal joints of the | 2 first interphalangeal joints, |
| thumb | and |
| 8 proximal interphalangeal | 8 proximal interphalangeal |
| joints, and | joints. |
| 8 distal interphalangeal joints. | |

The investigator will identify any joints to be excluded from evaluation at each visit. Replaced, synovectomized, ankylosed, or arthrodesed joints should be marked as non-evaluable.

Any joint that has had an intra-articular corticosteroid injection within 4 weeks prior to baseline should be excluded from evaluation during the study. The locations or a listing of these previous procedures should be documented in the participant's source documents or CRF.

The joint count will be assessed by scoring aspects of tenderness on pressure and passive movement of the particular joint. The participant will be asked for pain sensations on these manipulations and watched for spontaneous pain reactions. Any positive response on pressure, movement or both will be translated into a single tender-versus nontender dichotomy.

### Swollen Joint Counts

The number of swollen joints will be determined by examination of 66 joints, 33 joints on each side of the participant's body.

The 66 joints to be assessed and classified as swollen or not swollen are the same as those for the tender joint count except for the 2 hip joints.

Swelling is defined as palpable fluctuating synovitis of the joint.

The same method for marking as non-evaluable the replaced, synovectomized, ankylosed, or arthrodesed, and recently injected joints will apply.

### Physician Global Assessment of Disease Activity VAS (PhGADA_VAS)

The PhGADA_VAS is a single-item, clinician-reported outcome instrument that asks the physician to assess the participant's current disease activity, on a horizontal VAS of 0 to 100 mm:
0=no disease activity, and
100=extremely active disease.

The Safety Assessor will be responsible for completing the PhGADA_VAS after completion of the participant-reported assessments and the clinician-administered TSJC.

To ensure consistent PhGADA_VAS throughout the study, the instrument should be evaluated by the same physician at all study visits.

### Actigraphy

See Appendix 10.9.

### American College of Rheumatology (ACR) Criteria ACR20, ACR50, and ACR70

The ACR20, ACR50, and ACR70 are defined as at least 20%, 50%, and 70% improvement in the ACR core set values.

The percentage improvement in the ACR scores is determined by an improvement of at least 20%, 50%, or 70% in the number of TJC (0-68) and SJC (0-66) and an improvement of at least 20%, 50%, or 70% in at least 3 of these 5 assessments:
1. Patient's Assessment of Arthritis Pain VAS
2. PaGADA_VAS
3. PhGADA_VAS
4. HAQ-DI, or
5. Acute phase reactant as measured by hsCRP.

### Disease Activity Score-High-Sensitivity C-Reactive Protein (DAS28-hsCRP)

The DAS28-hsCRP measures disease activity in 28 joints using a composite numeric score of the following variables (Vander Cruyssen et al. 2005)
number of swollen joints (0 to 28)
number of tender joints (0 to 28)
hsCRP, and
PaGADA_VAS.

The 28 joints examined and assessed as tender or not tender for TJC and as swollen or not swollen for SJC include 14 joints on each side of the participant's body (Smolen et al. 1995):
2 shoulders
2 elbows
2 wrists
10 metacarpophalangeal joints
2 interphalangeal joints of the thumb
8 proximal interphalangeal joints, and
2 knees.

The DAS28-hsCRP remission is defined as DAS28-hsCRP <2.6.

The DAS28-hsCRP LDA is defined as DAS28-hsCRP≤3.2.

### Disease Activity Score—Erythrocyte Sedimentation Rate (DAS28-ESR)

The DAS28-ESR measures disease activity in 28 joints using a composite numeric score of the following variables (Vander Cruyssen et al. 2005):
TJC
SJC
ESR, and
PaGADA_VAS.

For the description of the 28 joints, see Section 8.1.12.

The DAS28-ESR remission is defined as DAS28-ESR <2.6.

The DAS28-ESR LDA is defined as DAS28-ESR≤3.2.

### Simplified Disease Activity Index (SDAI)

The SDAI is a tool for measurement of disease activity in RA that integrates measures of physical examination, acute phase response, patient self-assessment, and evaluator assessment (Aletaha and Smolen 2005). The SDAI is calculated by adding together scores from the following assessments:
number of swollen joints (0 to 28)
number of tender joints (0 to 28)
hsCRP in mg/dL (0.1 to 10.0)
PaGADA_VAS (0 to 100 mm), and
PhGADA VAS (0 to 100 mm).

For the description of the 28 joints, see Section 8.1.12.

Disease remission according to ACR/EULAR index-based definition of remission is defined as an SDAI score of ≤3.3 (Felson et al. 2011).

LDA is defined as an SDAI score of ≤11.

### Clinical Disease Activity Index (CDAI)

The CDAI is similar to the SDAI, but it allows for immediate scoring because it does not use a laboratory result (Aletaha and Smolen 2005). The CDAI is calculated by adding together scores from the following assessments
number of swollen joints (0 to 28)
number of tender joints (0 to 28)
PaGADA_VAS (0 to 100 mm), and
PhGADA VAS (0 to 100 mm).

For the description of the 28 joints, see Section 8 1.12.

Remission is defined as a CDAI score of ≤2.8 (Felson et al. 2011).

LDA is defined as a CDAI score of ≤10.

### MRI Imaging and Synovitis Score

MRI scans will be taken of the hand and wrist to determine the presence of synovitis, and the OMERACT RAMRIS system will determine the synovitis scores (østergaard et al. 2017).

Two assessors at a central vendor will read and score the screening and Week 12 scans in pairs for each participant. The assessors will be blinded to the participant's treatment and the temporal order of the scans. A detailed charter from the central reading laboratory will outline the MRI procedures, including image acquisition, image analysis, and data transfer.

### Safety Assessments

Planned time points for all safety assessments are provided in the SoA (Section 1.3).

### Physical Examinations

The physical examination will be either complete or symptom directed.

Investigators should pay special attention to clinical signs related to previous serious illnesses.

### Complete Physical Examination

A complete physical examination will include assessments of these areas and body systems
Peripheral lymph nodes
Thyroid palpation
Cardiovascular
Respiratory
Gastrointestinal, and
Neurologic.

### The Complete Physical Examination

excludes pelvic, rectal, and breast examinations, unless clinically indicated, and
includes TB assessments, as applicable (see “TB assessments” below and see Section 8.2 9).

A complete physical examination may be repeated at the investigator's discretion at any time a participant presents with physical complaints.

Symptom-Directed Physical Assessments after Screening

These assessments are performed based on participant status and standard of care.

### TB Assessments

At screening and approximately every 3 months thereafter, the specified physical evaluation, whether complete or symptom-directed, will include a documented assessment of TB risk factors and symptoms of signs of active TB, including an assessment of peripheral lymph nodes (see Section 8 2.9).

### Height and Weight

Height and weight will be measured and recorded.

### Vital Signs

Vital signs include body temperature, blood pressure, and pulse rate. Additional vital signs may be measured at the discretion of the investigator.

### Timing of Collection of Vital Signs

### Measure Vital Signs

after the participant has been sitting for at least 5 minutes, and
before obtaining an ECG tracing or collection of blood samples for laboratory testing.

### Electrocardiograms

### ECG Collection

For each participant, 12-lead digital ECGs in triplicate will be collected according to the SoA. ECGs must be recorded before collecting any blood samples. Participants must be supine for approximately 5 to 10 minutes before ECG collection and remain supine but awake during ECG collection.

ECGs (triplicate) will be obtained at approximately 1-minute intervals. ECGs may be obtained at additional times, when deemed clinically necessary.

ECG interpretation

ECGs will be interpreted by a qualified physician (the investigator or qualified designee) at the site as soon after the time of ECG collection as possible, and ideally while the participant is still present, to determine whether the participant meets entry criteria at the relevant visit(s) and for immediate participant management, should any clinically relevant findings be identified.

Participant Assessment after ECGs

If a clinically significant quantitative or qualitative change from baseline is identified after enrollment, the investigator will assess the participant for symptoms (for example, palpitations, near syncope, syncope) to determine whether the participant can continue in the study (Section 7.1.4). The investigator or qualified designee is responsible for determining if any change in participant management is needed and must document his/her review of one of the replicate ECGs printed at the time of collection

### Responsibilities of the Central ECG Laboratory

Digital ECGs will be electronically transmitted to a central ECG laboratory designated by the sponsor. A cardiologist at the central ECG laboratory will then conduct a full overread on 1 of the replicate ECGs, including all intervals. A report based on data from this overread will be issued to the investigative site. For each set of replicates, the RR and QT intervals and heart rate will be determined on the ECGs that were not fully overread. These data are not routinely reported back to the investigative site. All data from the overreads will be placed in the sponsor's database for analytical and study report purposes. Any clinically significant finding that was not present on the fully overread single ECG, but was present in the other replicate ECGs, will be reported to the investigator and to the sponsor.

If there are differences in ECG interpretation between the investigator (or qualified designee) and the cardiologist at the central ECG laboratory, the investigator's (or qualified designee's) interpretation will be used for study entry and immediate participant management. Interpretations from the cardiologist at the central ECG laboratory will be used for data analysis and report writing purposes.

The investigator (or qualified designee) must document his/her review of the final overread ECG report issued by the central ECG laboratory, and any alert reports.

### Chest Imaging

A high-quality, locally performed chest x-ray (posterior-anterior view and, if needed, a lateral view), interpreted and reported by a radiologist or pulmonologist, will be obtained as specified in the SoA.

For each participant, the chest x-ray films, images, or a radiology report must be available to the investigator for review before the participant is randomly assigned to a treatment in this study.

### Conditions for Using a Previous Chest X-Ray at Screening

Participants do not need to have a chest x-ray at screening if, in the opinion of the investigator, both of these 2 conditions are met:
the chest x-ray was performed within 3 months before the initial screening, and
documentation of the chest x-ray, read by a qualified radiologist or pulmonologist, is sufficient for TB evaluation according to local standard of care.

Note: In some jurisdictions, the interval between x-rays must be greater than 3 months. If so, a chest x-ray performed within 6 months before Visit 1 can be used.

### Alternatives to Chest X-Ray

In consultation with the sponsor's medical monitor, results of a chest CT scan or other imaging study similar to a chest x-ray, if performed within the same time window, may be used instead of a chest x-ray for the TB evaluation.

### Hepatic Safety Monitoring

### Close Hepatic Monitoring

Laboratory tests (Appendix 10.6), including ALT, AST, ALP, TBL, D. Bil, GGT, and CK, should be repeated within 48 to 72 hours to confirm the abnormality and to determine if it is increasing or decreasing, if 1 or more of these conditions occur:

| If a participant with baseline | |
|---|---|
| results of . . . | develops the following elevations: |
| ALT or AST <1.5× ULN | ALT or AST ≥3× ULN |
| ALP <1.5× ULN | ALP >2× ULN |
| TBL <1.5× ULN | TBL >2× ULN (except for patients |
| | with Gilbert's syndrome) |
| ALT or AST ≥1.5× ULN | ALT or AST >2× baseline |
| ALP >1.5× ULN | ALP >2× baseline |
| TBL ≥1.5× ULN | TBL ≥1.5× baseline (except for patients |
| | with Gilbert's syndrome) |

If the abnormality persists or worsens, clinical and laboratory monitoring, and evaluation for possible causes of abnormal liver tests should be initiated by the investigator in consultation with the Lilly-designated medical monitor. At a minimum, this evaluation should include physical examination and a thorough medical history, including symptoms, recent illnesses (for example, heart failure, systemic infection, hypotension, or seizures), recent travel, history of concomitant medications (including over-the-counter), herbal and dietary supplements, and history of alcohol drinking and other substance abuse.

Initially, monitoring of symptoms and hepatic biochemical tests should be done at a frequency of 1 to 3 times weekly, based on the participant's clinical condition and hepatic biochemical tests. Subsequently, the frequency of monitoring may be lowered to once every 1 to 2 weeks, if the participant's clinical condition and lab results stabilize. Monitoring of ALT, AST, ALP, and TBL should continue until levels normalize or return to approximate baseline levels.

### Comprehensive Hepatic Evaluation

A comprehensive evaluation should be performed to search for possible causes of liver injury if 1 or more of these conditions occur:

| If a participant with baseline | |
|---|---|
| [symbol] | develops the following elevations: |
| ALT or AST <1.5 × ULN | ALT or AST ≥3 × ULN with hepatic signs/symptoms^{a}, or |
| | ALT or AST ≥5 × ULN |
| ALP <1.5 × ULN | ALP ≥3 × ULN |
| TBL <1.5 × ULN | TBL ≥2 × ULN (except for patients with Gilbert's syndrome) |
| ALT or AST ≥1.5 × ULN | ALT or AST ≥2 × baseline with hepatic signs/symptoms^{a}, or |
| | ALT or AST ≥3 × baseline |
| ALP ≥1.5 × ULN | ALP ≥2 × baseline |
| TBL ≥1.5 × ULN | TBL ≥2 × baseline (except for patients with Gilbert's |
| | syndrome) |

^{a}Hepatic signs or symptoms are severe fatigue, nausea, vomiting, right upper quadrant abdominal pain, fever, rash, and/or eosinophilia >5%. [symbol]
indicates data missing or illegible when filed

At a minimum, this evaluation should include physical examination and a thorough medical history, as outlined above, as well as tests for PT-INR; tests for viral hepatitis A, B, C, or E; tests for autoimmune hepatitis; and an abdominal imaging study (for example, ultrasound or CT scan).

Based on the patient's history and initial results, further testing should be considered in consultation with the Lilly-designated medical monitor, including tests for HDV, CMV, EBV, acetaminophen levels, acetaminophen protein adducts, urine toxicology screen, Wilson's disease, blood alcohol levels, urinary ethyl glucuronide, and blood phosphatidylethanol. Based on the circumstances and the investigator's assessment of the participant's clinical condition, the investigator should consider referring the participant for a hepatologist or gastroenterologist consultation, magnetic resonance cholangiopancreatography, endoscopic retrograde cholangiopancreatography, cardiac echocardiogram, or a liver biopsy.

Additional Hepatic Data Collection (Hepatic Safety CRF) in Study Participants Who have Abnormal Liver Tests During the Study

Additional hepatic safety data collection in hepatic safety CRFs should be performed in study participants who meet 1 or more of the following 5 conditions:
1. Elevation of serum ALT to ≥5×ULN on 2 or more consecutive blood tests (if baseline ALT <1.5×ULN).
   In participants with baseline ALT ≥1.5×ULN, the threshold is ALT ≥3× baseline on 2 or more consecutive tests
2. Elevated TBL to ≥2×ULN (if baseline TBL <1.5×ULN) (except for cases of known Gilbert's syndrome).
   In participants with baseline TBL >1.5×ULN, the threshold should be TBL ≥2× baseline
3. Elevation of serum ALP to ≥2×ULN on 2 or more consecutive blood tests (if baseline ALP <1.5×ULN)
   In participants with baseline ALP ≥1.5×ULN, the threshold is ALP ≥2× baseline on 2 or more consecutive blood tests
4. Hepatic event considered to be an SAE
5. Discontinuation of study drug due to a hepatic event
   Note: the interval between the 2 consecutive blood tests should be at least 2 days.

### Tuberculosis Testing and Monitoring

### Screening

During screening, all participants are to be assessed for risk factors, symptoms, and

signs of TB with all of the following:
Thorough history to determine the lifetime risk factors for TB infection, for TB progression, and for symptoms and/or signs of active TB, and
Signs of previous or active TB by means of
   Thorough physical examination for signs of active TB, including measurement of body temperature (Section 8.2.2) and assessment of peripheral lymph nodes (Section 8 2.1), and
   A high-quality chest x-ray (posterior-anterior view, including a lateral view if needed) interpreted and reported by a radiologist or pulmonologist (Section 8.2.4).

All participants with no history of LTBI or active TB, and no history of positive Mantoux TST using PPD or positive M tuberculosis IGRA must have 1 of the following tests:
PPD TST, or
IGRA for M tuberculosis.

For details about these tests, see Appendix 10.7.

### Diagnosed LTBI

Participants diagnosed with LTBI are excluded (Section 5.2) unless they are candidates for LTBI treatment, are treated for LTBI, and the following criteria are met:
After receiving at least 4 weeks of appropriate LTBI therapy (as per WHO or the United States CDC guidelines), there is no evidence of hepatotoxicity (ALT/AST must remain ≤2 times ULN) or other treatment intolerance. In this case, the participant may be rescreened (Section 5 4) and is not excluded due to LTBI.
The participant must continue and complete appropriate LTBI therapy to remain eligible to continue to receive study intervention (Section 7.1.1).

### Monitoring During the Study

For all participants, monitoring for TB is to be continuous throughout the study. At a minimum, each participant is to have the following documented at least every 3 months:
Thorough history to determine any risk factors for TB infection and for TB progression, and symptoms or signs of active TB, and
Thorough physical examination for signs of active TB, including measurement of body temperature and assessment of peripheral lymph nodes (Sections 8.2.1 and 8.2.2).

### Hepatitis B Testing and Monitoring

As specified in the SoA, initial testing for HBV infection includes HBsAg and anti-HBc.
If HbsAg is positive, the participant is excluded.
If HbsAg is negative and anti-HBc is negative, the participant is not excluded.
If HbsAg is negative and anti-HBc is positive, further testing for HBV DNA is required.
   If the screening HBV DNA is positive, the participant is excluded.
   If the screening HBV DNA is negative, the participant is not excluded. Repeat testing for HBV DNA is required at least every 3 months during the study.
      Management of Enrolled Participants with Detectable HBV DNA During the Study

If HBV DNA is detected, the study intervention will be temporarily withheld or permanently discontinued, as described in Sections 7.1.1 and 7.1.2, and the participant should receive appropriate follow-up medical care from a hepatologist or other specialty physician with expertise in evaluation and management of viral hepatitis.

### Hepatitis C Testing

As specified in the SoA (Section 1.3), initial testing for HCV infection includes testing for anti-HCV.
If anti-HCV is positive, a test for circulating HCV RNA is required.
If HCV RNA test is negative, the participant is not excluded.
If HCV RNA test is positive, the participant is excluded (see Section 5.2).

Participants who have had HCV infection and have been successfully treated, defined as a sustained virologic response (HCV RNA by PCR negative for at least 24 weeks following treatment completion) are not excluded on the basis of HCV as long as HCV RNA test is negative at screening.

If HCV RNA is detected during the study, the study intervention will be discontinued, and the participant should receive appropriate follow-up medical care from a hepatologist or other specialty physician with expertise in evaluation and management of viral hepatitis (Section 7.1.2).

### Adverse Events, Serious Adverse Events, and Product Complaints

The definitions of the following events can be found in Appendix 10 3:
AEs
SAEs, and
Product complaints (PCs)

These events will be reported by the participant (or, when appropriate, by a caregiver, surrogate, or the participant's legally authorized representative).

The investigator and any qualified designees are responsible for detecting, documenting, and recording events that meet these definitions and remain responsible for following up events that are serious, considered related to the study intervention or study procedures, or that caused the participant to discontinue the study intervention or the study (see Section 7).

Care will be taken not to introduce bias when detecting events. Open-ended and non-leading verbal questioning of the participant is the preferred method to inquire about event occurrences.

After the initial report, the investigator is required to proactively follow each participant at subsequent visits/contacts. All SAEs and Aes of special interest (as defined in Section 8.3.3) will be followed until resolution, stabilization, the event is otherwise explained, or the participant is lost to follow-up (as defined in Section 7.3). For product complaints, the investigator is responsible for ensuring that follow-up includes any supplemental investigations as indicated to elucidate the nature and/or causality. Further information on follow-up procedures is provided in Appendix 10.3.

### Timing and Mechanism for Collecting Events

This table describes the timing, deadlines, and mechanism for collecting events.

| | | | | | |
|---|---|---|---|---|---|
| | | | Timing for | | |
| | | | Reporting to | | Back-up |
| | Collection | Collection | Sponsor or | Mechanism | Method of |
| Event | Start | Stop | Designee | for Reporting | Reporting |

Adverse Event

| | | | | | |
|---|---|---|---|---|---|
| AE | Signing of | Participation | As soon as | AE CRF | N/A |
| | the ICF | in study has | possible upon | | |
| | | ended | site awareness | | |

Serious Adverse Event

| | | | | | |
|---|---|---|---|---|---|
| SAE and | Signing of | Start of | Within 24 hours | SAE CRF | SAE |
| SAE updates - | the ICF | intervention | of awareness | | paper |
| prior to | | | | | form |
| start of study | | | | | |
| intervention | | | | | |
| and deemed | | | | | |
| reasonably | | | | | |
| possibly | | | | | |
| related to | | | | | |
| study | | | | | |
| procedures | | | | | |
| SAE and | Start of | Participation | Within 24 hours | SAE CRF | SAE |
| SAE updates - | intervention | in study has | of awareness | | paper |
| after start | | ended | | | form |
| of study | | | | | |
| intervention | | | | | |
| SAE^{a} - after | After | N/A | Promptly | SAE paper | N/A |
| participant's | participant's | | | form | |
| study | study | | | | |
| participation | participation | | | | |
| has ended | has ended | | | | |
| and the | | | | | |
| investigator | | | | | |
| becomes | | | | | |
| aware | | | | | |

Pregnancy

| | | | | | |
|---|---|---|---|---|---|
| Pregnancy in | After the | At least 28 | Within 24 hours | Pregnancy | Pregnancy |
| female | start of | days after | (see Section | paper form | paper |
| participants | study | the last dose | 8.3.2) | | form |
| and female | intervention | | | | |
| partners of | | | | | |
| male | | | | | |
| participants | | | | | |

Product Complaints

| | | | | | |
|---|---|---|---|---|---|
| PC | Start of | End of study | Within 24 hours | Product | N/A |
| associated | study | intervention | of awareness | Complaint | |
| with an SAE | intervention | | | form | |
| or might | | | | | |
| have led to | | | | | |
| an SAE | | | | | |
| PC not | Start of | End of study | Within 1 | Product | N/A |
| associated | study | intervention | business day of | Complaint | |
| with an SAE | intervention | | awareness | form | |
| Updated PC | — | — | As soon as | Originally | N/A |
| information | | | possible upon | completed | |
| | | | site awareness | Product | |
| | | | | Complaint | |
| | | | | form with all | |
| | | | | changes signed | |
| | | | | and dated by | |
| | | | | the investigator | |
| PC (if | Participation | N/A | Promptly | Product | |
| investigator | in study has | | | Complaint | |
| becomes | ended | | | form | |
| aware) | | | | | |

^{a}SAEs should not be reported unless the investigator deems them to be possibly related to study treatment or study participation.

### Adverse Events of Special Interest

AESIs for this study include
malignancies, and
serious infections, including opportunistic infections (Section 8.3.3.1) and tuberculosis (Section 8 2.9 and Appendix 10 7).

If these AESIs are reported, sites will be prompted to collect additional details and data.

No adjudication will be needed for these AESIs.

### Infections, Including Serious Infections and Opportunistic Infections

Completion of the Infection CRF page is required for each infection reported as an AE or SAE. The sponsor will identify infections considered to be opportunistic based on the article by Winthrop et al. 2015 (Appendix 10.8).

### Pharmacokinetics

Venous blood samples will be collected for measurement of plasma concentrations of Compound I as specified in the SoA.

A maximum of 3 samples may be collected at additional time points during the study if warranted and agreed upon between the investigator and the sponsor.

Instructions for the collection and handling of biological samples will be provided by the sponsor. The actual date and time (24-hour clock time) of each PK sample will be recorded, as well as the date and time of study intervention administration prior to the PK sample collection.

Samples will be used to evaluate the PK of Compound I. Samples collected for analyses may also be used to evaluate safety or efficacy aspects related to concerns arising during or after the study.

Intervention concentration information that may unblind the study will not be reported to investigative sites or blinded personnel.

### Bioanalysis

Samples will be analyzed at a laboratory approved by the sponsor and stored at a facility designated by the sponsor. Concentrations of Compound I will be assayed using a validated bioanalytical method. Analyses of samples collected from placebo-treated participants are not planned.

Sample retention is described in Appendix 10.1, Section 10.1.12. Remaining samples used for PK may be pooled and used for exploratory metabolism or bioanalytical method experiments as deemed appropriate.

### Pharmacodynamics

Samples for assessment of exploratory PD markers, including hsCRP, ESR, and calprotectin, will be collected at the times specified in the SoA (Section 1 3). Sample retention is described in Appendix 10.1, Section 10.1.12.

### Biomarkers

Blood samples will be collected for exploratory biomarker research (Appendix 10.2).

The visits and times for collecting biomarker samples are specified in the SoA (Section 1.3).

Samples will be stored and analysis may be performed for research purposes on the drug target, disease process, pathways associated with disease state, mechanism of action of Compound I, or research methods or in validating diagnostic tools or assay(s) related to rheumatoid arthritis.

Biomarker research is performed to address questions of relevance to drug disposition, target engagement, PD, mechanism of action, variability of participant's response (including safety), and clinical outcome. Sample collection is incorporated into clinical studies to enable examination of these questions through measurement of biomolecules, including DNA, RNA, proteins, lipids, and other cellular elements.

All samples will be coded with the participant number. These samples and any data generated can be linked back to the participant only by the investigative site personnel.

Samples will be retained at a facility selected by the sponsor or its designee. The maximum duration of retention is described in Appendix 10.1, Section 10.1.12

### Statistical Considerations

The statistical analysis plan will be finalized prior to unblinding, and it will include a more technical and detailed description of the statistical analyses described in this section. This section is a summary of the planned statistical analyses of the most important endpoints, including primary and key secondary endpoints.

### Statistical Hypotheses

### Phase 2a

The primary objective is to demonstrate that Compound I is superior to placebo in mean change from baseline in DAS28-hsCRP at Week 12. Thus, the null hypothesis to be tested in relation to the primary estimand is
There is no difference between Compound I and placebo in participants with moderately-to-severely active RA, as measured by mean change in DAS28-hsCRP from baseline to Week 12.

### Phase 2b

The primary objective is to demonstrate that Compound I is superior to placebo in achieving ACR50 at Week 12. Thus, the null hypothesis to be tested in relation to the primary estimand is
There is no difference between Compound I and placebo in achieving ACR50 in participants with moderately-to-severely active RA, as measured by the proportion of study participants achieving ACR50 at Week 12.

### Multiplicity Adjustment

Adjustment for multiple comparisons will not be employed in the analysis for this study.

### Analyses Sets

The following populations are defined for the study:

| Participant Analysis Set | Description |
|---|---|
| Modified intent to treat | All participants randomly assigned to study intervention and |
| (mITT) | who take at least 1 dose of study intervention. Participants will |
| | be analyzed according to the intervention to which they were |
| | assigned. |
| Safety | All participants randomly assigned to study intervention and |
| | who take at least 1 dose of study intervention. Participants will |
| | be analyzed according to the intervention they actually received |
| | within each study period. |
| Pharmacokinetic (PK) | All participants randomly assigned to study intervention and |
| | who take at least 1 dose of study intervention and have PK data |
| | available. |

Efficacy analyses will be conducted on the mITT population, unless otherwise specified, and will combine efficacy data collected in Phase 2a and Phase 2b, as appropriate. Separate efficacy analyses by study phases may be provided on the mITT population if deemed necessary. Safety summaries will be provided on the safety population by study phases and by treatment periods. More detailed definitions of populations will be provided in the SAP.

### Statistical Analyses

### General Considerations

Statistical analysis of this study will be the responsibility of the sponsor or its designee. A detailed SAP describing the statistical methodologies will be developed by the sponsor or its designee.

Handling of missing, unused, and spurious data is addressed prospectively in the overall statistical methods described in the protocol and in the SAP, where appropriate. Adjustments to the planned analyses will be described in the final CSR.

Efficacy and safety data will be analyzed and summarized by phases and treatment periods if appropriate. For details about the phases and periods of the study, see Section 4.1.

Efficacy analyses will be conducted on the mITT population, unless otherwise specified. Safety analyses will be conducted on the safety population.

For Phase 2b efficacy analysis in Study Period II, Phase 2a efficacy data collected in Study Period II will be combined, as appropriate, to assist with the Phase 2b analysis. More details will be provided in the SAP.

Baseline for each treatment period is defined as the last nonmissing assessment on or prior to the date of the first study intervention. Any assessment collected after the first dosing is defined as postbaseline for the treatment period. Change from baseline will be calculated as the visit value of interest minus the baseline value. If a baseline value or the value at the visit is missing for a particular variable, then the change from baseline is defined as missing.

Summary statistics for continuous variables may include mean, standard deviation, median, and minimum and maximum values.

Categorical variables will be presented as counts and percentages. Variables will be analyzed in the original scale on which they are measured, unless otherwise specified.

The parametric approach will be employed by default for statistical analysis except when nonparametric analysis, such as by a rank-based method, is more fitting.

Additional exploratory analyses of the data will be conducted as deemed appropriate.

Dichotomous responder endpoints, including the primary endpoint, will be analyzed using a logistic regression model with treatment group, baseline disease activity, and stratification factors as covariates. The odds ratio and p-value based on odds ratio will be reported. The 95% CIs of mean difference will also be reported without being adjusted by covariates. Missing data will be imputed using the nonresponder imputation method.

Treatment comparisons of continuous efficacy endpoints with multiple postbaseline timepoints will be analyzed using MMRM analysis. The MMRM model will include
treatment
stratification factors
baseline value
visit and treatment-by-visit interaction in the model as fixed factors, and
participant as a random factor.

The covariance structure to model the within-participant errors will be unstructured. If the unstructured covariance matrix results in a lack of convergence, the heterogeneous Toeplitz covariance structure, followed by the heterogeneous autoregressive covariance structure, will be used.

The Kenward-Roger method will be used to estimate the denominator degrees of freedom. Type III sums of squares for the LS means will be used for the statistical comparison. The 95% CI will also be reported. Missing data will be handled with the missing at random assumption. No additional imputation methods will be applied to the MMRM analysis.

Where appropriate, treatment comparisons of continuous efficacy endpoints will be analyzed using ANCOVA modeled with treatment group, stratification factors, and baseline value as covariates. Type III sums of squares for the LS means will be used for statistical comparison between treatment groups. The LS mean difference, standard error, p-value, and 95% CI, unless otherwise specified, will also be reported. Missing data imputation method for the ANCOVA model will be the last observation carried forward.

For Phase 2b study analysis, dose response modeling will be performed on the primary objective to assist in dose selection decisions. More details will be provided in the SAP.

Any change to the data analysis methods or imputation methods described in the protocol will require an amendment only if it changes a principal feature of the protocol. Any other change to the data analysis methods described in the protocol, and the justification for making the change, will be described in the SAP and the CSR. Additional exploratory analyses of the data may be conducted as deemed appropriate.

### Primary Endpoint/Estimand Analyses

### Phase 2a

The primary endpoint is the change in DAS28-hsCRP from baseline to Week 12 for the Compound I treatment group compared to placebo; it will be analyzed using the MMRM model. The model will include treatment, stratification factors, baseline value, visit, and treatment-by-visit interaction in the model as fixed factors, and participant as a random factor. See Section 9.3 1 for details on the methods.

A hypothetical estimand strategy is proposed for intercurrent events, including discontinuing study intervention, taking prohibited medications, and changing in permitted concomitant therapy. A likelihood-based method under missing at random assumption will be used to handle the missing data.

### Phase 2b

The primary endpoint is the proportion of participants achieving ACR50 at Week 12 for the Compound I treatment group compared to placebo; it will be analyzed using logistic regression adjusted by stratification factors and baseline disease activity. Baseline DAS28-hsCRP score will be used as the baseline variable. See Section 9.3.1 for details on the methods to be used to test the differences between each active treatment arm and placebo.

Relevant data collected in Phase 2a will be combined with Phase 2b data for the analysis.

A composite strategy is proposed for other ICEs, including discontinuing study intervention, taking prohibited medications, and changes in permitted concomitant therapy. Participants will be considered nonresponders for visits after those ICEs occur. The nonresponder imputation will be used for missing data.

### Secondary Endpoint Analyses

The secondary efficacy and health outcome endpoints at Week 12 will be analyzed using the statistical analysis methods described in Section 9.3.1.

For the secondary efficacy endpoints at Week 24, summary statistics will be provided as described in Section 9.3.1.

Additional details will be provided in the SAP.

### Pharmacokinetics/Pharmacodynamics

Compound I plasma concentrations will be illustrated graphically and summarized descriptively. If warranted and based on availability of data, the exposure-response relationship of plasma Compound I concentrations to efficacy endpoints and/or safety endpoints may be explored. A model-based approach may be implemented to estimate PK or PD parameters.

### Exploratory Analyses

Exploratory analyses may be further described in the SAP that will be finalized before database lock.

### Safety Analyses

Safety analyses will be assessed by evaluating exposure, AEs, laboratory analytes, vital signs, and adverse events of special interests.

Duration of exposure to therapy during the treatment periods will be calculated for each participant and summarized by treatment group.

The AEs will be coded according to the MedDRA and summarized by system organ class, preferred term, severity, and relationship to the study intervention. All AEs, including pre-existing conditions, will be listed by participant, visit, preferred term, treatment group, severity, and relationship to the treatment.

A TEAE is defined as an event that first occurred or worsened in severity after baseline, with baseline defined as all pre-existing conditions recorded at Visit 1 and any AEs recorded before the first dose of study intervention (that is, during the interval between Visits 1 and 2, and recorded with the time of onset before the first dose of study intervention). The treatment period will be used as the postbaseline period for the analysis. For events that are gender specific, the denominator and computation of the percentage will include only participants from the given gender.

The number and percentage of participants who reported TEAEs, TEAEs by maximum severity, deaths, SAEs, TEAEs related to study intervention, discontinuations from the treatment due to an AE, and AESIs will be summarized. TEAEs (all, by maximum severity), SAEs including deaths, and AEs that lead to treatment discontinuation will be summarized and analyzed by MedDRA system organ class and preferred term.

Treatment-related TEAEs (that is, TEAEs related to study intervention) are defined as events that are indicated by the investigator on the CRF to be related to treatment.

AESIs or special safety topics will be identified by a standardized MedDRA query or a sponsor-defined MedDRA-preferred term listing.

Follow-up emergent AEs, SAEs including deaths, and AEs that lead to study discontinuation will be summarized. All AEs, including pre-existing conditions, will be listed by participant, visit, preferred term, treatment group, severity, and relationship to the treatment.

### Other Analyses

### Subgroup Analyses

Summary of subgroups will be provided. Subgroup analyses may be conducted for the primary endpoints (Phase 2a: change from baseline in DAS28-hsCRP; Phase 2b: response rate in ACR50 at Week 12). Subgroups that may be evaluated include previous RA therapy use, gender, race, geographic region, and disease duration.

Detailed description of the summaries and/or statistical analyses will be provided in the SAP.

### Interim Analyses

Analyses for the primary database locks for Phase 2a and Phase 2b will be conducted as described in Section 9.3, when all participants in each phase have completed the Week 12 visit or have discontinued from study intervention.

### Potential Prespecified Interim Analyses

Any of the predefined interim analyses may be conducted at the discretion of the sponsor.

Phase 2a: The first and second interim analyses prior to the analysis of the primary database lock may be conducted when approximately 30% to 50% and 50% to 70% of participants have completed Week 12 or have discontinued from study intervention, respectively. The purpose of the interim analyses will be to support planning activities associated with Compound I clinical development program.

A higher dose level cohort, not to exceed a maximum of 125 mg, may be added during Phase 2a based on the results of the interim analyses and recommendation of the sponsor's IAC. Compound I treatment doses for Phase 2b may be based on the interim analysis results and recommendations of the sponsor's IAC (Section 10.1.5).

The interim analyses for Phase 2a may assess safety, PK, and/or efficacy measures. No adjustment of type I error will be performed.

Phase 2b: The first and second interim analyses prior to the analysis of the primary database lock may be conducted when approximately 30% to 50% and 50% to 70% of participants have completed Week 12 or have discontinued from study intervention, respectively. The purpose of the interim analyses will be to support planning activities associated with Compound I clinical development program. The interim analyses for Phase 2b may assess safety, PK, and/or efficacy measures. No adjustment of type I error will be performed.

Relevant Phase 2a data will be used for interim analyses in Phase 2b.

Other interim analyses may be conducted at the discretion of the sponsor. All interim analyses will be used to support planning activities associated with the clinical development program.

### Assessment of Unblinded Interim Data

Assessment of unblinded interim data will be conducted by an IAC with a limited number of prespecified team members who do not have direct site contact or data entry or validation responsibilities (see Section 10.1.5). Only the IAC will be authorized to evaluate unblinded interim efficacy and safety analyses. Study sites will receive information about interim results only if they need to know for the safety of their participants.

Prior to the interim or final database lock, a limited number of preidentified individuals may gain access to the unblinded data to initiate the final population PK/PD model development processes for interim or final analyses.

To minimize bias, the SAP and PK/PD analysis plan will be finalized and approved before any unblinding.

Unblinding details will be specified in a separate unblinding document. Information that may unblind the study during the analyses will not be reported to study sites or to the blinded study team until the prespecified milestone for unblinding of study results. Study sites will receive information about interim results only if they need to know for the safety of their participants.

### Sample Size Determination

### Phase 2a

Approximately 100 participants will be randomly assigned to Compound I 50 mg and placebo in a ratio of 2:1. All randomly assigned participants in the mITT population will be considered evaluable.

The power calculations for Phase 2a portion of the study assume the following:
At Week 12, the Compound I 50 mg group will have a sample size of approximately 67 participants, and the placebo group will have a sample size of approximately 33 participants.
It is assumed that the DAS28-hsCRP changes from baseline for Compound I 50 mg and placebo group are −1.80 and −0.85, respectively, with standard deviation equal to 1.25.
The power is calculated using 2-sided test with an error rate of α=0.05.

Given these assumptions, the power to reject the null hypothesis is 89%.

Additional cohort in Phase 2a

For additional cohort, approximately up to 100 participants may be randomly assigned to a higher dose level cohort of Compound I, not to exceed a maximum of 125 mg, and placebo in a ratio of 2:1. All randomly assigned participants in the mITT population will be considered evaluable. Participants assigned to placebo group in the previous cohort will be combined.

The power calculations for additional cohort in Phase 2a portion of the study assume the following:
At the end of study, 50, 75, and 100 patients will be additionally randomized.
A sample size of approximately 33 participants assigned to placebo group in the previous cohort will be pooled for the calculation.
The DAS28-hsCRP changes from baseline for Compound I higher dose level and placebo group are −1.80 and −0.85, respectively, with standard deviation equal to 1.25.
The power is calculated using 2-sided test with an error rate of α=0.05.

The powers to reject the null hypotheses for the treatment groups with additional sample sizes equal to 50, 75, 100 are 84%, 94%, and 97%, respectively.

### Phase 2b

Approximately 280 participants will be randomly assigned to 1 of the following 4 treatment groups in a 2:1:2:2 ratio:
125 mg QD Compound I
50 mg QD Compound I
25 mg QD Compound I, or
placebo.

All randomly assigned participants in the mITT population will be considered evaluable.

The power calculations for Phase 2b study assume the following:
At Week 12, the 125 mg dose of Compound I, the 25 mg dose of Compound I, and placebo groups will each have a sample size of approximately 80 participants, and the 50 mg dose of Compound I will have a sample size of approximately 40 participants.
For simplicity in the power calculation, it is assumed that all the 3 Compound I treatment groups will have the same treatment effect.
It is assumed that the true placebo response rate in ACR50 at Week 12 is approximately 10% and the true treatment response in ACR50 at Week 12 is approximately 32%.
The power is calculated using pairwise 2-sided test with an error rate of α=0.05.

Given these assumptions, the power to reject the null hypothesis for the Compound I treatment groups with sample sizes equal to 40 and 80 compared to a placebo group with sample size of 80 is 82% and 94%, respectively.

Abbreviations and Definitions

| Term | Definition |
|---|---|
| abuse | use of a study intervention for recreational purposes or |
| | to maintain an addiction or dependence |
| ACR | American College of Rheumatology |
| ACTH | adrenocorticotropic hormone |
| AE | adverse event |
| AESI | adverse event of special interest |
| ALP | alkaline phosphatase |
| ALT | alanine aminotransferase |
| ANC | absolute neutrophil count |
| ANCOVA | analysis of covariance |
| anti-HBC | hepatitis B core antibody |
| APTT | activated partial thromboplastin time |
| AST | aspartate aminotransferase |
| AUC | area under the plasma concentration - time curve |
| authorized IMP | Applicable to the EU only: a medicinal product authorized in |
| | accordance with Regulation (EC) No 726/2004 or in any Member State |
| | concerned in accordance with Directive 2001/83/EC, irrespective of |
| | changes to the labeling of the medicinal product, which is used as an |
| | investigational medicinal product |
| BCG | Bacillus Calmette-Guerin |
| BCRP | breast cancer resistance protein |
| bDMARD | biologic disease-modifying antirheumatic drug |
| blinding/masking | A single-blind study is one in which the investigator and/or the |
| | investigator's staff are aware of the treatment but the participant is not, |
| | or vice versa, or when the sponsor is aware of the treatment but the |
| | investigator and/the investigator's staff and the participant are not. |
| | A double-blind study is one in which neither the participant nor any of |
| | the investigator or sponsor staff who are involved in the treatment or |
| | clinical evaluation of the subjects are aware of the treatment received. |
| BMI | body mass index |
| C-SSRS | Columbia Suicide-Severity Rating Scale |
| CBD | cannabidiol |
| CDAI | Clinical Disease Activity Index |
| CDC | [US] Centers for Disease Control and Prevention |
| CFR | Code of Federal Regulations |
| CI | confidence interval |
| CK | creatinine kinase |
| CKD-EPI | Chronic Kidney Disease Epidemiology Collaboration |
| CMV | cytomegalovirus |
| COA | clinical outcome assessments |
| Companion | An in vitro diagnostic device (assay or test) that provides information |
| diagnostic | that is essential for the safe and effective use of a corresponding |
| | therapeutic product |
| complaint | A complaint is any written, electronic, or oral communication that |
| | alleges deficiencies related to the identity, quality, purity, durability, |
| | reliability, safety or effectiveness, or performance of a drug or drug |
| | delivery system. |
| compliance | Adherence to all study-related, good clinical practice (GCP), and |
| | applicable regulatory requirements |
| CONSORT | Consolidated Standards of Reporting Trials |
| CRF | case report form; a printed, optical, or electronic document designed to |
| | record all of the protocol-required information to be reported to the |
| | sponsor for each trial participant |
| csDMARD | conventional synthetic disease-modifying antirheumatic drug |
| CSR | clinical study report |
| CT | computed tomography |
| CXR | chest x-ray |
| CYP | cytochrome P450 |
| D. Bil | direct bilirubin |
| DAS28-hsCRP | Disease Activity Score - high-sensitivity C-reactive protein |
| DAS28-ESR | Disease Activity Score-Erythrocyte Sedimentation Rate |
| DDI | drug-drug interaction |
| DHEA-S | dehydroepiandrosterone sulfate |
| Device | equivalent to product complaint |
| deficiencies | |
| DMARD | disease-modifying antirheumatic drug |
| DO | doctor of osteopathic medicine |
| EBV | Epstein-Barr virus |
| eCOA | electronic clinical outcome assessments |
| ECG | electrocardiogram |
| EDC | electronic data capture |
| eGFR | estimated glomerular filtration rate |
| enroll | The act of assigning a participant to a treatment. Participants who are |
| | enrolled in the study are those who have been assigned to a treatment. |
| enter | Participants entered into a study are those who sign the informed |
| | consent form directly or through their legally acceptable |
| | representatives. |
| ESR | erythrocyte sedimentation rate |
| EULAR | European League Against Rheumatism |
| FACIT-F | Functional Assessment of Chronic Illness Therapy-Fatigue |
| FSH | follicle-stimulating hormone |
| GCP | good clinical practice |
| GGT | gamma-glutamyltransferase |
| HAQ-DI | Health Assessment Questionnaire - Disability Index |
| HBsAg | hepatitis B surface antigen |
| HBV | hepatitis B virus |
| HCV | hepatitis C virus |
| HDV | hepatitis D virus |
| HIV | human immunodeficiency virus |
| hsCRP | high-sensitivity C-reactive protein |
| IAC | Internal Assessment Committee |
| IB | Investigator's Brochure |
| ICE | intercurrent event |
| ICF | informed consent form |
| ICH | International Council for Harmonisation |
| IEC | Independent Ethics Committees |
| IGRA | interferon gamma release assay |
| IMP | Investigational Medicinal Product (see also “investigational product”) |
| | A medicinal product that is being tested or used as a reference, |
| | including as a placebo, in a clinical trial. |
| informed consent | A process by which a participant voluntarily confirms their willingness |
| | to participate in a particular study, after having been informed of all |
| | aspects of the study that are relevant to the participant's decision to |
| | participate. Informed consent is documented by means of a written, |
| | signed and dated informed consent form. |
| INR | international normalized ratio |
| interim analysis | An interim analysis is an analysis of clinical study data, separated into |
| | treatment groups, that is conducted before the final reporting database |
| | is created/locked. |
| investigational | A pharmaceutical form of an active ingredient or placebo being tested |
| product | or used as a reference in a clinical trial, including products already on |
| | the market when used or assembled (formulated or packaged) in a way |
| | different from the authorized form, or marketed products used for an |
| | unauthorized indication, or marketed products used to gain further |
| | information about the authorized form. See also “IMP.” |
| IRB | Institutional Review Boards |
| ITT | intention to treat: The principle that asserts that the effect of a treatment |
| | policy can be best assessed by evaluating on the basis of the intention |
| | to treat a participant (that is, the planned treatment regimen) rather than |
| | the actual treatment given. It has the consequence that participant |
| | allocated to a treatment group should be followed up, assessed, and |
| | analyzed as members of that group irrespective of their compliance to |
| | the planned course of treatment. |
| IV | intravenous |
| IWRS | interactive web-response system |
| LDA | low disease activity |
| LS | least squares |
| LTBI | latent tuberculosis infection |
| MAD | multiple ascending dose |
| MD | medical doctor |
| medication error | Errors in the prescribing, dispensing, or administration of a study |
| | intervention, regardless of whether or not the medication is |
| | administered to the participant or the error leads to an AE. Medication |
| | error generally involves a failure to uphold 1 or more of the 5 “rights” |
| | of medication use: the right participant, the right drug, the right dose, |
| | right route, at the right time. |
| | In addition to the core 5 rights, the following may also represent |
| | medication errors: |
| | dose omission associated with an AE or a product complaint |
| | dispensing or use of expired medication |
| | use of medication past the recommended in-use date |
| | dispensing or use of an improperly stored medication |
| | use of an adulterated dosage form or administration technique |
| | inconsistent with the medication's labeling (for example, Summary |
| | of Product Characteristics, IB, local label, protocol), or |
| | shared use of cartridges, prefilled pens, or both. |
| misuse | use of a study intervention for self-treatment that either is inconsistent |
| | with the prescribed dosing regimen, indication, or both, or is obtained |
| | without a prescription |
| mITT | modified intent to treat |
| MMRM | mixed-effects for repeated measures |
| MRI | magnetic resonance imaging |
| MTX | methotrexate |
| NOAEL | nonhuman primate study with a no-observed-adverse-effect-level |
| NP | nurse practitioner |
| NRS | numeric rating scale |
| NSAIDs | nonsteroidal anti-inflammatory drugs |
| OATP | organic anion transporting polypeptide |
| OCT1 | organic cation transporter 1 |
| P-gp | P-glycoprotein |
| PaGADA | Patient's Global Assessment of Disease Activity |
| participant | Equivalent to CDISC term “subject”: an individual who participates in |
| | a clinical trial, either as recipient of an investigational medicinal |
| | product or as a control |
| PC | product complaint |
| PCR | polymerase chain reaction |
| PD | pharmacodynamics |
| PhGADA | Physician's Global Assessment of Disease Activity |
| PK/PD | pharmacokinetics/pharmacodynamics |
| PPD | purified protein derivative |
| PPS | per-protocol set: The set of data generated by the subset of participant |
| | who sufficiently complied with the protocol to ensure that these data |
| | would be likely to exhibit the effects of treatment, according to the |
| | underlying scientific model. |
| PRO/ePRO | patient-reported outcomes/electronic patient-reported outcomes |
| PT | prothrombin time |
| QD | once daily |
| QTc | corrected QT interval |
| QTL | quality tolerance limits |
| RA | rheumatoid arthritis |
| RAMRIS | Rheumatoid Arthritis Magnetic Resonance Imaging Score |
| RIPK1 | receptor-interacting protein kinase 1 |
| RN | registered nurse |
| RNA | ribonucleic acid |
| SAD | single ascending dose |
| SAE | serious adverse event |
| SAP | statistical analysis plan |
| screen | the act of determining if an individual meets minimum requirements to |
| | become part of a pool of potential candidates for participation in a |
| | clinical study |
| SDAI | Simplified Disease Activity Index |
| SERMs | selective estrogen receptor modulators |
| SF-36 | Short Form-36 version 2 health survey acute form |
| SJC | swollen joint count |
| SoA | Schedule of Activities |
| SOC | standard of care |
| SUSAR | suspected unexpected serious adverse reaction |
| T3 | triiodothyronine |
| T4 | thyroxine |
| TB | tuberculosis |
| TBL | total bilirubin level |
| TBNK | T cells, B cells, and natural killer cells |
| TEAE | treatment-emergent adverse event: An untoward medical occurrence |
| | that emerges during a defined treatment period, having been absent |
| | pretreatment, or worsens relative to the pretreatment state, and does not |
| | necessarily have to have a causal relationship with this treatment. |
| TJC | tender joint count |
| TNF | tumor necrosis factor |
| TSH | thyroid stimulating hormone |
| TST | tuberculin skin test |
| tsDMARD | Targeted synthetic DMARDs include, but are not limited to, Janus |
| | kinas (JAK) inhibitors, tofacitinib and baricitinib. |
| ULN | upper limit of normal |
| VAS | visual analog scale |
| WHO | World Health Organization |
| WNOCBP | women not of childbearing potential |
| WOCBP | women of childbearing potential |

### Example 5

### Abbreviated Protocol:

A Phase 2, Randomized, Double-Blind, Placebo-Controlled Study to Investigate the Efficacy and Safety of Compound I in Adults with Moderate-to-Severe Plaque Psoriasis

### Brief Title:

A Study to Investigate the Efficacy and Safety of Compound I Administered Orally Compared with Placebo in Adult Participants Aged 18 to 75 with Moderate-to-Severe Plaque Psoriasis

### Rationale:

This study aims to investigate the impact of Compound I on clinical outcomes in participants with moderate-to-severe plaque psoriasis. This is the first Phase 2 study and data from this study will inform decisions for the clinical development of Compound I.

### Objectives, Endpoints, and Estimands:

| Objectives | Endpoints |
|---|---|
| Primary | |
| To evaluate if Compound I | Proportion of participants who |
| is superior to placebo for | achieve 75% or more resolution of |
| the treatment of moderate- | psoriasis (PASI 75) at Week 12 |
| to-severe plaque psoriasis | |
| in adults | |
| Secondary | |
| Compare the clinical | Proportion of participants who |
| response of Compound I | achieve 90% or more resolution of |
| to placebo | psoriasis (PASI 90) at Week 12 |
| | Proportion of participants who |
| | achieve complete resolution of |
| | psoriasis (PASI 100) at Week 12 |
| | Mean change from baseline at |
| | Week 12 in reduction of body |
| | surface area (BSA) with psoriasis |
| | Proportion of participants achieving |
| | sPGA 0 or sPGA 0/1 (clear or |
| | almost clear) at Week 12 |
| | PSSI mean change from baseline to |
| | Week 12 for participants with |
| | baseline scalp measures |
| Characterize the | Plasma concentrations of |
| pharmacokinetics | Compound I |
| (PK) of Compound I | |
| Compare patient-reported | Mean change from baseline at |
| outcomes from participants | Week 12 for quality-of-life |
| that received Compound I | measures |
| to those that received | DLQI |
| placebo | PatGA, and |
| | PSS |

Abbreviations: DLQI = Dermatology Life Quality Index; PASI = Psoriasis Area and Severity Index; PASI 75 = 75% improvement in PASI from baseline; PASI 90 = 90% improvement in PASI from baseline; PASI 100 = 100% improvement in PASI from baseline; PatGA = Patient's Global Assessment of Psoriasis; PSS = Psoriasis Symptoms Scale; sPGA = Static Physician's Global Assessment.

### Estimand

The primary clinical question of interest is
What is the difference in PASI 75 after 12 weeks of intervention between Compound I and placebo in adult participants with moderate-to-severe plaque psoriasis if participants who discontinue study intervention due to lack of efficacy or adverse events, or use prohibited medication are considered as non-responders?

### Brief Summary

### Screening

Interested participants will sign the appropriate informed consent document(s) prior to completion of any procedures.

The investigator will review symptoms, risk factors, and other non-invasive inclusion and exclusion criteria prior to any invasive procedures. If the participant is eligible after this review, then the site will perform the invasive procedures to confirm eligibility.

### Double-Blind Treatment and Assessment Period

This is the general sequence of events during the treatment and assessment period:
Complete baseline procedures and sample collection
Participants are randomized to an intervention group
Participants receive study intervention, and
Complete all efficacy assessments, safety monitoring, and post-dosing sample collection.

### Post-Treatment Follow-Up

Post-treatment follow-up assessments will be conducted at 4 weeks after the last dose to assess clinical status and for adverse events (AEs).

### Number of Participants:

Approximately 125 participants will be screened to achieve approximately 105 who are randomly assigned to study intervention.

### Intervention Groups and Duration:

There are 3 intervention groups: Compound I 50 mg, Compound I 125 mg, and placebo.

Dosing is once daily (QD).

Data Monitoring Committee: Yes

An internal assessment committee will review the interim efficacy and safety data in an unblinded fashion.

### Schedule of Activities (SoA)

| | | Double-Blind Treatment | | | | | | | | | Follow- | |
|---|---|---|---|---|---|---|---|---|---|---|---|---|
| Study | Screening | Period | | | | | | | | | up | Notes |
| Visits | 1 | 2 | 3 | 4 | 5 | 6 | 7 | 8 | ED | 997 | 801 | ED = early |
| | | | | | | | | | | | | discontinuation |
| | | | | | | | | | | | | 997 = unscheduled |
| | | | | | | | | | | | | visit |
| Weeks from | −4 | — | 2 | 4 | 6 | 8 | 10 | 12 | — | — | 16 | |
| randomization | | | | | | | | | | | | |
| Study day | ≤28 days | 1 | 15 | 29 | 43 | 57 | 71 | 85 | — | — | | |
| | from V2 | | | | | | | | | | | |
| Visit interval | | — | ±2 | ±2 | ±2 | ±2 | ±2 | ±2 | — | — | ±7* | Visit 1 procedures |
| tolerance (days) | | | | | | | | | | | | may be conducted |
| | | | | | | | | | | | | over more than 1 |
| | | | | | | | | | | | | day if all activities |
| | | | | | | | | | | | | are completed |
| | | | | | | | | | | | | within the |
| | | | | | | | | | | | | allowable visit |
| | | | | | | | | | | | | tolerance. |
| | | | | | | | | | | | | *V801 occurs |
| | | | | | | | | | | | | either 4 weeks after |
| | | | | | | | | | | | | ED or Visit 8. |

Procedures

| | | | | | | | | | | | |
|---|---|---|---|---|---|---|---|---|---|---|---|
| Informed consent | X | | | | | | | | | | The informed |
| | | | | | | | | | | | consent form (ICF) |
| | | | | | | | | | | | must be signed |
| | | | | | | | | | | | before any |
| | | | | | | | | | | | protocol-specific |
| | | | | | | | | | | | tests/procedures are |
| | | | | | | | | | | | performed. |
| Inclusion and | X | X | | | | | | | | | |
| exclusion criteria, | | | | | | | | | | | |
| review and | | | | | | | | | | | |
| confirm | | | | | | | | | | | |
| Demographics | X | | | | | | | | | | Includes ethnicity |
| | | | | | | | | | | | (United States |
| | | | | | | | | | | | only), year of birth, |
| | | | | | | | | | | | gender, and race. |
| Preexisting | X | | | | | | | | | | |
| conditions and | | | | | | | | | | | |
| medical history, | | | | | | | | | | | |
| including | | | | | | | | | | | |
| relevant surgical | | | | | | | | | | | |
| history | | | | | | | | | | | |
| Prespecified | | | | | | | | | | | Includes psoriasis |
| medical history | | | | | | | | | | | diagnosis, onset, |
| (indication and | | | | | | | | | | | and last clinically |
| history of | | | | | | | | | | | significant flare; |
| interest) | | | | | | | | | | | comorbidities such |
| | | | | | | | | | | | as diabetes, |
| | | | | | | | | | | | coronary artery |
| | | | | | | | | | | | disease, stroke, |
| | | | | | | | | | | | IBD, and psoriatic |
| | | | | | | | | | | | arthritis. |
| Prior treatments | X | | | | | | | | | | All prior treatments |
| for indication | | | | | | | | | | | for psoriasis. |
| Substance use | | X | | | | | | | | | |
| (alcohol, | | | | | | | | | | | |
| caffeine, tobacco) | | | | | | | | | | | |
| Concomitant | X | X | X | X | X | X | X | X | X | X | |
| medications | | | | | | | | | | | |
| Adverse events | X | X | X | X | X | X | X | X | X | X | Any events that |
| (AEs) | | | | | | | | | | | occur after signing |
| | | | | | | | | | | | the informed |
| | | | | | | | | | | | consent are |
| | | | | | | | | | | | considered AEs as |
| | | | | | | | | | | | defined in Section |
| | | | | | | | | | | | 8.3.1. Additional |
| | | | | | | | | | | | data will be |
| | | | | | | | | | | | collected for |
| | | | | | | | | | | | infection-related |
| | | | | | | | | | | | AEs (Section |
| | | | | | | | | | | | 8.3.3). |

Physical
Evaluations

| Height | X | | | | | | | | | | | Participant should |
|---|---|---|---|---|---|---|---|---|---|---|---|---|
| | | | | | | | | | | | | remove shoes. |
| Weight | X | X | | | | | | X | X | | X | |
| Vital signs | X | X | X | X | X | X | X | X | X | X | X | Includes blood |
| | | | | | | | | | | | | pressure, body |
| | | | | | | | | | | | | temperature, and |
| | | | | | | | | | | | | pulse rate. |
| | | | | | | | | | | | | Vital signs should |
| | | | | | | | | | | | | be measured after |
| | | | | | | | | | | | | participant has been |
| | | | | | | | | | | | | sitting at least 5 |
| | | | | | | | | | | | | minutes, before |
| | | | | | | | | | | | | obtaining an ECG |
| | | | | | | | | | | | | tracing, and before |
| | | | | | | | | | | | | collection of blood |
| | | | | | | | | | | | | samples for |
| | | | | | | | | | | | | laboratory testing. |
| Physical | X | | | | | | | | | | | See Section 8.2.2. |
| examination | | | | | | | | | | | | This physical |
| | | | | | | | | | | | | examination |
| | | | | | | | | | | | | includes assessment |
| | | | | | | | | | | | | of tuberculosis |
| | | | | | | | | | | | | (TB) risk factors |
| | | | | | | | | | | | | and symptoms or |
| | | | | | | | | | | | | signs of TB, |
| | | | | | | | | | | | | including an |
| | | | | | | | | | | | | assessment of |
| | | | | | | | | | | | | peripheral lymph |
| | | | | | | | | | | | | nodes. |
| | | | | | | | | | | | | The complete |
| | | | | | | | | | | | | physical |
| | | | | | | | | | | | | examination |
| | | | | | | | | | | | | excludes pelvic, |
| | | | | | | | | | | | | rectal, and breast |
| | | | | | | | | | | | | examinations, |
| | | | | | | | | | | | | unless clinically |
| | | | | | | | | | | | | indicated. |
| Symptom- | | X | X | X | X | X | X | X | X | X | X | See Section 8.2.2. |
| directed physical | | | | | | | | | | | | The symptom- |
| examination | | | | | | | | | | | | directed physical |
| | | | | | | | | | | | | examination |
| | | | | | | | | | | | | includes assessment |
| | | | | | | | | | | | | of tuberculosis |
| | | | | | | | | | | | | (TB) risk factors |
| | | | | | | | | | | | | and symptoms or |
| | | | | | | | | | | | | signs of TB, |
| | | | | | | | | | | | | including an |
| | | | | | | | | | | | | assessment of |
| | | | | | | | | | | | | peripheral lymph |
| | | | | | | | | | | | | nodes. See Section |
| | | | | | | | | | | | | 8.2.8. |
| TB monitoring | | | | | | | | X | | | | See Sections 8.2.2 |
| and thyroid | | | | | | | | | | | | and 8.2.8. |
| palpation | | | | | | | | | | | | |
| 12-lead ECG | X | X | | | | | | X | X | | | Locally performed. |
| | | | | | | | | | | | | ECGs should be |
| | | | | | | | | | | | | recorded prior to |
| | | | | | | | | | | | | collection of blood |
| | | | | | | | | | | | | samples for any |
| | | | | | | | | | | | | laboratory testing. |
| | | | | | | | | | | | | ECGs may be |
| | | | | | | | | | | | | obtained at |
| | | | | | | | | | | | | additional times, |
| | | | | | | | | | | | | when deemed |
| | | | | | | | | | | | | clinically |
| | | | | | | | | | | | | necessary. See |
| | | | | | | | | | | | | Section 8.2.3. |
| Chest X-ray | X | | | | | | | | | | | See Section 8.2.4. |
| posterior-anterior | | | | | | | | | | | | Not done at |
| and lateral view | | | | | | | | | | | | screening if |
| | | | | | | | | | | | | done within |
| | | | | | | | | | | | | 3 months |
| | | | | | | | | | | | | before |
| | | | | | | | | | | | | screening, |
| | | | | | | | | | | | | and |
| | | | | | | | | | | | | documentation |
| | | | | | | | | | | | | of the |
| | | | | | | | | | | | | previous |
| | | | | | | | | | | | | chest X-ray is |
| | | | | | | | | | | | | available. |
| | | | | | | | | | | | | Lateral view may |
| | | | | | | | | | | | | also be taken. |
| | | | | | | | | | | | | Locally performed. |
| | | | | | | | | | | | | Interpreted and |
| | | | | | | | | | | | | reported by |
| | | | | | | | | | | | | radiologist or |
| | | | | | | | | | | | | pulmonologist. |
| | | | | | | | | | | | | All of these |
| | | | | | | | | | | | | assessments need to |
| Patient- | | | | | | | | | | | | be collected before |
| Reported | | | | | | | | | | | | the clinician- |
| Outcomes | | | | | | | | | | | | administered |
| (Electronic) | | | | | | | | | | | | assessments. |
| DLQI | | X | | X | | X | | X | X | | X | Dermatology Life |
| | | | | | | | | | | | | Quality Index |
| PatGA | | X | | X | | X | | X | X | | X | Patient's Global |
| | | | | | | | | | | | | Assessment of |
| | | | | | | | | | | | | Psoriasis |
| PSS | | X | | X | | X | | X | X | | X | Psoriasis Symptoms |
| | | | | | | | | | | | | Scale |
| QIDS | | X | | X | | X | | X | | | X | Quick Inventory of |
| | | | | | | | | | | | | Depressive |
| | | | | | | | | | | | | Symptomatology |

Clinician-Administered Assessments (Electronic)

| | | | | | | | | | | | |
|---|---|---|---|---|---|---|---|---|---|---|---|
| PASI | X | X | X | X | X | X | X | X | X | X | Psoriasis Area and |
| | | | | | | | | | | | Severity Index |
| Body surface | X | X | X | X | X | X | X | X | X | X | Percent |
| area | | | | | | | | | | | involvement of |
| | | | | | | | | | | | psoriasis on each |
| | | | | | | | | | | | participant's body |
| | | | | | | | | | | | surface |
| sPGA | X | X | X | X | X | X | X | X | X | X | Static Physician's |
| | | | | | | | | | | | Global Assessment |
| PSSI | | X | | | | | | X | X | | Psoriasis Scalp |
| | | | | | | | | | | | Severity Index. |
| | | | | | | | | | | | Only for |
| | | | | | | | | | | | participants with |
| | | | | | | | | | | | scalp involvement |
| | | | | | | | | | | | at baseline. |

Clinician-Administered Assessments (Paper)

| | | | | | | | | | | | | |
|---|---|---|---|---|---|---|---|---|---|---|---|---|
| C-SSRS | X | | | | | | | | | | | |
| Screening | | | | | | | | | | | | |
| C-SSRS Since | | X | X | X | X | X | X | X | X | X | X | Adapted for the |
| Last Visit | | | | | | | | | | | | assessment of |
| | | | | | | | | | | | | ideation and |
| | | | | | | | | | | | | behavior categories |
| | | | | | | | | | | | | only. See |
| | | | | | | | | | | | | Section 8.2.10.1 for |
| | | | | | | | | | | | | details. |
| Fitzpatrick Skin | | X | | | | | | | | | | See Section 8.2.11. |
| Type | | | | | | | | | | | | |

Laboratory Tests and Sample Collection

| | | | | | | | | | | | | |
|---|---|---|---|---|---|---|---|---|---|---|---|---|
| Hematology | X | X | X | X | X | X | X | X | X | X | X | |
| Clinical | X | X | X | X | X | X | X | X | X | X | X | |
| chemistry | | | | | | | | | | | | |
| Troponin | | X | | | | | | | X | | X | |
| Urinalysis | X | | X | X | X | X | X | X | X | X | X | |
| Serum pregnancy | X | | | | | | | | | | | |
| Urine pregnancy | | X | | | | | | | X | | X | Locally performed. |
| Follicle- | | | | | | | | | | | | Optional, perform |
| stimulating | | | | | | | | | | | | only to confirm |
| hormone (FSH) | | | | | | | | | | | | postmenopausal |
| | | | | | | | | | | | | status. |
| eGFR (CKD- | X | X | X | X | X | X | X | X | X | | X | Calculated by Lilly- |
| EPI) | | | | | | | | | | | | designated |
| | | | | | | | | | | | | laboratory. |
| TB test | X | | | | | | | X | | | | See Section 8.2.8. |
| Human | X | | | | | | | | | | | |
| immunodeficiency | | | | | | | | | | | | |
| virus (HIV) | | | | | | | | | | | | |
| screening tests | | | | | | | | | | | | |
| Hepatitis C virus | X | | | | | | | | | | | If HCV antibody |
| (HCV) screening | | | | | | | | | | | | test is positive, it |
| tests | | | | | | | | | | | | must be followed |
| | | | | | | | | | | | | by an HCV RNA |
| | | | | | | | | | | | | test. |
| | | | | | | | | | | | | See Section 8.2.7. |
| Hepatitis B virus | X | | | | | | | | | | | Includes testing for |
| (HBV) screening | | | | | | | | | | | | HBsAg and anti- |
| tests | | | | | | | | | | | | HBc. |
| | | | | | | | | | | | | See Section 8.2.7. |
| HBV DNA | X | | | | | | | X | | | | Only for |
| | | | | | | | | | | | | participants who |
| | | | | | | | | | | | | are positive for |
| | | | | | | | | | | | | anti-HBc at |
| | | | | | | | | | | | | screening. See |
| | | | | | | | | | | | | Section 8.2.7. |
| C-reactive | | X | | X | | X | | X | X | | X | |
| protein, high- | | | | | | | | | | | | |
| sensitivity | | | | | | | | | | | | |
| (hsCRP) | | | | | | | | | | | | |
| IL-19 | | X | X | X | | X | | X | X | | X | For each sample |
| | | | | | | | | | | | | collected, record |
| | | | | | | | | | | | | time and date of |
| | | | | | | | | | | | | previous dose |
| | | | | | | | | | | | | administration. |
| Pharmacokinetic | | X | X | X | | X | | X | X | | | For each PK sample |
| (PK) samples | | | | | | | | | | | | collected, record |
| | | | | | | | | | | | | time and date of |
| | | | | | | | | | | | | previous dose |
| | | | | | | | | | | | | administration. |
| | | | | | | | | | | | | Collect PK samples |
| | | | | | | | | | | | | at these times: |
| | | | | | | | | | | | | Visit 2: 0.5 to 2 |
| | | | | | | | | | | | | hours post-dose |
| | | | | | | | | | | | | Visits 3 and 4: |
| | | | | | | | | | | | | Predose and 0.5 to |
| | | | | | | | | | | | | 2 hours post-dose |
| | | | | | | | | | | | | Visit 6: Predose |
| | | | | | | | | | | | | Visit 8: Predose |
| | | | | | | | | | | | | and immediately |
| | | | | | | | | | | | | prior to leaving site. |

Stored Samples

| | | | | | | | |
|---|---|---|---|---|---|---|---|
| Skin punch | X | | X | | X | | Visit 2: 1 biopsy |
| | | | | | | | each of lesional and |
| | | | | | | | non-lesional skin. |
| | | | | | | | Visits 4 and 8: 1 |
| | | | | | | | biopsy of lesional |
| | | | | | | | skin. |
| Genetics sample | X | | | | | | Sample can be |
| | | | | | | | obtained at or after |
| | | | | | | | Visit 2. |
| Exploratory | X | X | X | X | X | X | Visit 2: collect |
| biomarker | | | | | | | sample before |
| samples | | | | | | | dosing. |

Randomization and Dosing

| | | | | | | | | | | | |
|---|---|---|---|---|---|---|---|---|---|---|---|
| Register visit in | X | X | X | X | X | X | X | X | | X | |
| IWRS | | | | | | | | | | | |
| Randomization | | X | | | | | | | | | |
| Dispense study | | X | X | X | X | X | X | | | | |
| intervention to | | | | | | | | | | | |
| participant via | | | | | | | | | | | |
| IWRS | | | | | | | | | | | |
| Observe | | X | X | X | X | X | X | X | | | |
| participant | | | | | | | | | | | |
| administer study | | | | | | | | | | | |
| intervention at | | | | | | | | | | | |
| clinic | | | | | | | | | | | |
| Participant | | | X | X | X | X | X | X | X | | Participant is |
| returns unused | | | | | | | | | | | expected to bring |
| study | | | | | | | | | | | unused intervention |
| intervention | | | | | | | | | | | back to the clinic. |
| Assess study | | | X | X | X | X | X | X | X | | |
| intervention | | | | | | | | | | | |
| compliance | | | | | | | | | | | |

Abbreviations: anti-HBc = hepatitis B core antibody; C-SSRS = Columbia Suicide-Severity Rating Scale; CKD-EPI = Chronic Kidney Disease Epidemiology Collaboration [equation]; ECG = electrocardiogram; eGFR = estimated glomerular filtration rate; HBsAg = hepatitis B surface antigen; IL-19 = interleukin-19; IWRS = interactive web-response system.

### Objectives, Endpoints, and Estimands

| Objectives | Endpoints |
|---|---|
| Primary | |
| To evaluate if Compound I is superior to | Proportion of participants who achieve |
| placebo for the treatment of moderate-to- | 75% or more resolution of psoriasis |
| severe plaque psoriasis in adults | (PASI 75) at Week 12 |
| Secondary | |
| Compare the clinical response of | Proportion of participants who achieve |
| Compound I to placebo | 90% or more resolution of psoriasis |
| | (PASI 90) at Week 12 |
| | Proportion of participants who achieve |
| | complete resolution of psoriasis (PASI |
| | 100) at Week 12 |
| | Mean change from baseline at Week 12 |
| | in reduction of body surface area (BSA) |
| | with psoriasis |
| | Proportion of participants achieving |
| | sPGA 0 or sPGA 0/1 (clear or almost |
| | clear) at Week 12 |
| | PSSI mean change from baseline to |
| | Week 12 for participants with baseline |
| | scalp measures |
| Characterize the pharmacokinetics (PK) | Plasma concentrations of Compound I |
| of Compound I | |
| Compare patient-reported outcomes from | Mean change from baseline at Week 12 |
| participants that received Compound I to | for quality-of-life measures |
| those that received placebo | DLQI |
| | PatGA, and |
| | PSS |
| Exploratory | |
| To explore the effect of Compound I on | Clinical endpoints, IL-19, select |
| clinical endpoints or biomarkers | biomarkers |
| Compare the clinical response and | Clinical response and patient-reported |
| patient-reported outcomes of Compound | outcomes at timepoints collected through |
| I to placebo | Week 12 |
| To assess the relationship between | Plasma concentrations of Compound I, |
| Compound I exposure and clinical | PASI 75, IL-19, safety assessments |
| endpoints or biomarkers | |

Abbreviations: DLQI = Dermatology Life Quality Index; IL-19 = interleukin-19; PASI = Psoriasis Area and Severity Index; PASI 75 = 75% improvement in PASI from baseline; PASI 90 = 90% improvement in PASI from baseline; PASI 100 = 100% improvement in PASI from baseline; PatGA = Patient's Global Assessment of Psoriasis; PSS = Psoriasis Symptoms Scale; PSSI = Psoriasis Scalp Severity Index; sPGA = Static Physician's Global Assessment.

### Estimands

### Primary Clinical Question of Interest

The primary clinical question of interest is
What is the difference in PASI 75 after 12 weeks of intervention between Compound I and placebo in adult participants with moderate-to-severe plaque psoriasis if participants who discontinue study intervention due to lack of efficacy or adverse events, or use prohibited medication are considered as non-responders?

### Rationale for the Estimand

This estimand assumes that if a participant discontinued treatment due to an AE or lack of efficacy, the burden of treatment outweighed its benefits. It also assumes that if a participant violated the concomitant medication rules, the participant was not receiving sufficient benefit from treatment. Therefore, being a responder, or showing clinical improvement, requires a positive response as well as completing treatment and not violating concomitant medication rules.

### Estimand Attributes

This table describes the estimand attributes.

| | | | Intercurrent |
|---|---|---|---|
| | | | Events Related to |
| Objective | Endpoint | Estimand Strategy | Treatment |
| Primary | Dichotomous: PASI 75 | The intercurrent events | Early |
| Efficacy | | related to study | discontinuation |
| Secondary | Dichotomous: | intervention will be | of treatment |
| Efficacy | PASI 90 | handled through the | due to lack of |
| | PASI 100 | endpoint definition | efficacy |
| | sPGA(0) | (composite variable | Early |
| | sPGA(0/1) | strategy). Participants who | discontinuation |
| | Continuous: | meet these definitions will | of treatment |
| | PASI percent change from | be considered non- | due to adverse |
| | baseline | responders, or having no | events |
| | BSA mean change from | change from baseline for | Prohibited |
| | baseline | continuous endpoints. | medication use |
| | PSSI change from baseline | All remaining intercurrent | before Week 12 |
| Secondary | Continuous: | events will be handled | |
| PRO | DLQI mean change from | using a treatment policy | |
| | baseline | strategy. | |
| | PSS mean change from | | |
| | baseline | | |
| | PatGA mean change from | | |
| | baseline | | |

Abbreviations: DLQI = Dermatology Life Quality Index; PASI = Psoriasis Area and Severity Index; PASI 75 = 75% improvement in PASI from baseline; PASI 90 = 90% improvement in PASI from baseline; PASI 100 = 100% improvement in PASI from baseline; PatGA = Patient's Global Assessment of Psoriasis; PSS = Psoriasis Symptoms Scale; PSSI = Psoriasis Scalp Severity Index; sPGA = Static Physician's Global Assessment.

### Study Design

### Overall Design

This is a Phase 2, randomized, double-blind, placebo-controlled study to investigate the effectiveness and safety of Compound I in adults with moderate-to-severe plaque psoriasis.

This is a 12-week study with a follow-up visit 4 weeks after the last dose of intervention.

There are 3 intervention groups: Compound I 50 mg, Compound I 125 mg, and placebo.

Dosing is QD.

Additional participants may be added at the discretion of the sponsor to evaluate different dose levels and treatment durations to further study the safety, efficacy and other parameters outlined in the SoA. If these changes occur, the protocol will be amended and submitted to regulatory agencies and IRBs. Modifications to the study will be reviewed with the study sites prior to implementation.

### Design Outline

### Screening

Interested participants will sign the appropriate informed consent document(s) prior to completion of any procedures.

The investigator will review symptoms, risk factors, and other non-invasive inclusion and exclusion criteria prior to any invasive procedures. If the participant is eligible after this review, then the site will perform the invasive procedures to confirm eligibility.

### Double-Blind Treatment and Assessment Period

This is the general sequence of events during the treatment and assessment period:
Complete baseline procedures and sample collection
Participants are randomized to an intervention group
Participants receive study intervention, and
Complete all efficacy assessments, safety monitoring, and post-dosing sample collection.

### Post-Treatment Follow-Up

Post-treatment follow-up assessments will be conducted at 4 weeks after the last dose to assess clinical status and for AEs.

### Scientific Rationale for Study Design

### Primary Endpoint

PASI 75 is commonly used as a primary endpoint in clinical trials of moderate-to-severe plaque psoriasis.

### Overall Design

The 12-week duration of the treatment period is based on the 3-month toxicology results. It is also a reasonable timeframe to observe efficacy for the treatment of psoriasis.

The follow up at 4 weeks after the last dose is designed to capture any additional safety signals and assess time to potential relapse.

Placebo is chosen as the control treatment to assess whether any observed effects are treatment related or simply reflect the study conditions. The double-blind, randomized, placebo-controlled design minimizes bias on safety and tolerability assessments, and allows a more robust comparison among Compound I doses and placebo.

### End of Study Definition

The end of the study is defined as the date of the last scheduled procedure shown in the SoA for the last participant in the study globally.

A participant is considered to have completed the study if the participant has completed all periods of the study including the last scheduled procedure shown in the SoA.

### Study Population

Prospective approval of protocol deviations to recruitment and enrollment criteria, also known as protocol waivers or exemptions, is not permitted.

### Inclusion Criteria

Participants are eligible to be included in the study only if all of the following criteria apply:

### Age

1. Participant must be 18 to 75 years of age inclusive, at the time of signing the informed consent.

### Sex and Contraceptive Requirements

2. Are men who agree to use highly effective/effective methods of contraception or women not of childbearing potential.
Contraceptive use by participants should be consistent with local regulations regarding the methods of contraception for those participating in clinical studies.
Definitions and contraception requirements for participants in this study are provided in Section 10.4.

### Type of Participant and Disease Characteristics

3. Participants who have moderate-to-severe chronic plaque psoriasis for at least 6 months prior to baseline based on investigator-confirmed diagnosis of chronic psoriasis vulgaris, with these criteria
a. Plaque psoriasis involving ≥10% body surface area (BSA) and absolute Psoriasis Area and Severity Index (PASI) score ≥12 in affected skin at screening Visit 1 and randomization/baseline Visit 2.
b. Static Physician's Global Assessment (sPGA) score of ≥3 at Visit 1 and Visit 2.

### Study Procedures

4. Are willing and able to undergo punch biopsies.

5. Have venous access sufficient to allow for blood sampling.

6. Are able to swallow oral medication.

7. Are reliable and willing to make themselves available for the duration of the study and are willing to follow study procedures.

### Weight

8. Have a body mass index (BMI) within the range of 18 to 40 kg/m² (inclusive).

### Informed Consent

9. Capable of giving signed informed consent as described in Section 10.1, which includes compliance with the requirements and restrictions listed in the informed consent form (ICF) and in this protocol.

### Laboratory Test Results

10. Have clinical laboratory test results within normal reference range or results with acceptable deviations that are judged as not clinically significant by the investigator. This table outlines laboratory test results with required ranges for inclusion in this study.

| Test | Result |
|---|---|
| Hematology | |
| Absolute neutrophil | ≥1.5 × 10⁹/L(≥1.5 × 10³/μL or ≥1.5 GI/L) |
| count | |
| Platelet count | ≥lower limit of normal |
| Hemoglobin level | ≥10.0 g/dL |
| Lymphocyte count | >800 cells/μL (>0.80 × 10³/μL or >0.80 GI/L) |
| Total leukocyte count | ≥3.0 × 10/L (≥3.0 × 10³/μL or ≥3.0 GI/L) |
| Clinical Chemistry | |
| Serum creatinine, ALT, | levels ≤2 × upper limit of normal (ULN) |
| and AST | |
| TBL and ALP | <1.5 × ULN, participants with Gilbert's syndrome must have |
| | serum direct bilirubin <1.5 mg/dL |

### Exclusion Criteria

Participants are excluded from the study if any of the following criteria apply:

### Medical Conditions

11. Did not have a primary response (respond within first 12 weeks of treatment) to treatment with most recent TNF-α antagonist, per investigator assessment.

12. Have a clinically significant flare of psoriasis during the 12 weeks before baseline.

13. Have any other skin conditions, excluding plaque psoriasis, that would affect interpretation of data, including, but not limited to, scleroderma, eczema, drug-induced psoriasis, guttate psoriasis, pustular psoriasis, parapsoriasis, or cutaneous as judged by the investigator.

14. Diagnosis of immune-mediated conditions that are commonly associated with psoriasis for which a participant requires current systemic (oral, subcutaneous, or intravenous) immunosuppressant treatment (including corticosteroids and biologics).

15. Manifestations of other autoimmune diseases, such as systemic lupus erythematosus.

### Infections and Infectious Disease

16. Have a current or recent acute, active infection.
From 30 days prior to screening until the randomization/baseline visit, participants must have no significant symptoms including fever of 100.5° F. (38° C.) or above, or
From 60 days prior to screening until the randomization/baseline visit, participants must have no signs of confirmed or suspected infection, and must have completed any appropriate anti-infective treatment.

17. Had any of these types of infections within 3 months prior to screening and up to randomization/baseline visit
Serious: requiring hospitalization, or intravenous or equivalent oral antibiotic treatment
Opportunistic: as defined in Winthrop et al. 2015 (see Section 10.8)
Chronic: duration of symptoms, signs or treatment of 6 weeks or long
Recurring: including, but not limited to herpes simplex, herpes zoster, recurring cellulitis, chronic osteomyelitis
   Herpes zoster is considered active and ongoing until all vesicles are dry and crusted over
   Participants with only recurrent, mild and uncomplicated orolabial herpes, or genital herpes, or both, may be discussed with the sponsor's designated medical monitor and may be considered for enrollment if other eligibility criteria are met.

18. Have human immunodeficiency virus (HIV) infection.

19. Have a current infection with hepatitis B virus (HBV) that is, positive for hepatitis B surface antigen (HBsAg) and/or PCR positive for HBV DNA (see Section 8.2 7).

20. Have a current infection with hepatitis C virus (HCV) that is, positive for HCV RNA (see Section 8.2 7).

21. Have or have had active tuberculosis (TB) (see Section 8.2.8).

22. Have or have had latent TB infection (LTBI) that has not been treated with a complete course of appropriate therapy as defined by the World Health Organization (WHO) or the United States Centers for Disease Control and Prevention (CDC), unless such treatment is completed (see Section 8.2.8).

### Other Medical Conditions

23. Have clinically significant ECG abnormalities including corrected QT interval, Fridericia's correction >450 msec for males and >470 msec for females.

24. Have a history of additional risk factors for Torsades de Pointes such as, heart failure, hypokalemia, or a family history of long QT syndrome.

25. Have clinically relevant abnormal blood pressure (BP) or heart rate (HR) as determined by the investigator.

26. Have an unstable or uncontrolled illness, including but not limited to a cerebro-cardiovascular, respiratory, hepatic, renal, gastrointestinal, endocrine, hematologic, or neurologic disease, congestive heart failure, multiple sclerosis, or abnormal laboratory values at screening, that in the opinion of the investigator would potentially affect participant safety within the study or interfere with the interpretation of data.

27. Have a diagnosis or history of malignant disease within 5 years prior to baseline

### Exceptions

a. Basal cell or squamous epithelial carcinomas of the skin that have been resected with no evidence of metastatic disease for 3 years.
b. Cervical carcinoma in situ, with no evidence of recurrence within 5 years prior to baseline.

28. Have a history of or current significant psychiatric disorders.

29. Are actively suicidal and deemed a significant risk for suicide in the judgment of the investigator.

30. Have answered “yes” to either Question 4 or Question 5 on the “Suicidal Ideation” portion of the Columbia Suicide-Severity Rating Scale (C-SSRS) or
have answered “yes” to any of the suicide-related behaviors on the “suicidal behavior” portion of the C-SSRS, and
the ideation or behavior occurred within the past month.

31. Have a history of major surgery within 12 weeks prior to screening or will require major surgery during the study.

32. Have a history of significant allergies to lidocaine or its derivatives used during skin biopsy.

### Vaccines

33. Have received live vaccine(s), including live attenuated vaccines, within 4 weeks prior to screening or intend to receive during the study and is within 5 half-lives after the last dose of intervention.

### Exceptions

a. Non-live or inactivated vaccine if received at least 2 weeks before baseline or after the last study visit
b. Inactivated influenza and pneumococcal vaccines, and
c. SARS-COV-2 vaccines authorized by local regulatory bodies.

34. Have received a Bacillus Calmette-Guerin vaccine or treatment within 4 weeks prior to screening or intend to during the study and within less than 5 half-lives after the last dose of intervention.

### Prior/Concomitant Therapy

35. Have received any investigational intervention within 4 weeks or 5 half-lives prior to screening, whichever is longer.

36. Have a history of any non-psoriatic disease that required treatment with oral or parenteral corticosteroids for more than 2 weeks within 24 weeks prior to screening.

37. Have received biologic treatments for immune conditions, such as monoclonal antibodies, including marketed drugs, within 12 weeks or 5 half-lives prior to baseline, whichever is longer.

38. Have received systemic nonbiologic treatment for immune conditions within 4 weeks prior to baseline (see Section 10 7.2).

39. Have received topical psoriasis treatment within 14 days prior to baseline (see Section 10.7.2).

40. Are unable or unwilling to avoid excessive sun exposure or use of tanning booths for at least 4 weeks prior to baseline and during the study.

41. Currently using or plan to use medications that prolong the QT/QTc interval, except if they are used to treat depression.

42. Are using medications that are strong CYP3A4 inhibitors or inducers within 14 days prior to baseline or plan to use these medications during the study (see Section 10.7.2).

43. Plan to receive treatment with medications that are sensitive CYP3A substrates or P-gp substrates with a narrow therapeutic index (see Section 10.7.2).

### Prior/Concurrent Clinical Study Experience

44. Are currently enrolled in any other clinical study involving an investigational product or any other type of medical research judged not to be scientifically or medically compatible with this study.

### Other Exclusions

45. Have a history of chronic alcohol abuse, IV drug abuse, or other illicit drug abuse within 1 year prior to screening.

46. Have donated blood of more than 500 mL within 4 weeks prior to screening.

47. Have received blood products within 6 months prior to screening.

48. Are Lilly employees or are employees of any third party involved in the study who require exclusion of their employees.

49. Are investigator site personnel directly affiliated with this study and/or their immediate families. Immediate family is defined as a spouse, parent, child, or sibling, whether biological or legally adopted.

50. Are unsuitable for inclusion in this study in the opinion of the investigator.

### Study Intervention(s) and Concomitant Therapy

Study intervention is defined as any investigational intervention(s), marketed product(s), placebo, or medical device(s) intended to be administered to/used by a study participant according to the study protocol.

### Study Intervention(s) Administered

Participants will take the tablets orally, QD, approximately every 24 hours, with or without food.

| Treatment Name | Compound I | Compound I | | Placebo |
|---|---|---|---|---|
| Dose Form | tablet | tablet | | tablet |
| Unit Dose | 25 mg | 25 | mg | placebo |
| Strength(s) | | | | |
| Dosage Level(s) | 50 mg | 125 | mg | — |

| | | | |
|---|---|---|---|
| Route of | oral | oral | oral |
| Administration | | | |
| Use | experimental | experimental | placebo |

| | |
|---|---|
| Sourcing | Study intervention will be provided |
| | centrally by the sponsor or its designee. |
| Labeling | Study intervention will be labeled as |
| | required per country requirement. |

### Measures to Minimize Bias: Randomization and Blinding

### Randomization

All participants will be centrally assigned to randomized study intervention using an interactive web-response system (IWRS). Before the study is initiated, the log in information and directions for the IWRS will be provided to each site.

Participants will be stratified according to prior treatment with biologics for treatment of psoriasis.

Study intervention will be dispensed at the study visits summarized in SoA. Each participant will be dispensed the same number of tablets to maintain the blind. Returned study intervention should not be re-dispensed to the participants.

### Emergency Unblinding

The IWRS will be programmed with blind-breaking instructions. In case of an emergency, the investigator has the sole responsibility for determining if unblinding of a participant's intervention assignment is warranted. Participant safety must always be the first consideration in making such a determination.

If a participant's intervention assignment is unblinded, the sponsor must be notified immediately within 24 hours of this occurrence. The date and reason that the blind was broken must be recorded.

Discontinuation from the Study in Case of Unblinding

If an investigator, site personnel performing assessments, or participant is unblinded, the participant must be discontinued from the study (Section 7.2). In cases where there are ethical reasons to have the participant remain in the study, the investigator must obtain specific approval from a sponsor or designee for the participant to continue in the study.

### Study Intervention Compliance

Participant compliance with study intervention will be assessed at each visit by counting returned tablets.

A participant will be considered significantly noncompliant if they miss more than 20% of the prescribed doses of study intervention during the study, unless the participant's study intervention is withheld by the investigator for safety reasons. Similarly, a participant will be considered significantly noncompliant if they are judged by the investigator to have intentionally or repeatedly taken 20% more than the prescribed amount of medication during the study.

Participants will be counseled by study staff on the importance of taking the study intervention as prescribed, as appropriate.

A record of the number of tablets dispensed to and taken by each participant must be maintained and reconciled with study intervention and compliance records. Intervention start and stop dates, including dates for intervention delays will also be recorded in the case report form (CRF).

### Dose Modification

This protocol does not allow dose adjustments.

Continued Access to Study Intervention after the End of the Study

Study intervention will not be available to participants after completion of the study.

### Concomitant Therapy

See Section 10.7 for lists of medications that are permitted or prohibited in this study.

Any medication or vaccine, including over-the-counter or prescription medicines, vitamins, and/or herbal supplements, that the participant is receiving at the time of enrollment or receives during the study must be recorded along with
Reason for use
Dates of administration including start and end dates, and
Dosage information including dose and frequency.

All participants should maintain their usual medication regimens for concomitant conditions or diseases throughout the study, unless those medications are specifically excluded in the protocol.

Participants taking concomitant medications should be on stable dosages at the time of baseline and should remain at stable dosages throughout the study, unless changes need to be made because of AEs.

Participants should consult with authorized site personnel before taking any new medications or supplements during the study. Authorized site personnel should consult the sponsor's medical monitor if there are any questions about concomitant therapies during the study.

Preliminary in vitro data suggests that Compound I is a weak inducer of CYP3A. Investigators should be aware of participants' concomitant use of CYP3A substrates.

### Discontinuation of Study Intervention and Participant Discontinuation/Withdrawal

Discontinuation of specific sites or of the study are handled in Section 10.19.

### Discontinuation of Study Intervention

Study intervention may be permanently discontinued or temporarily withheld during the study.

### Liver Chemistry Stopping Criteria

The study intervention should be interrupted or discontinued if 1 or more of these conditions occur.

| Elevation | Exception |
|---|---|
| ALT or AST >8 × ULN | |
| ALT or AST >5 × ULN for more than 2 weeks | |
| ALT or AST >3 × ULN and either TBL >2 × ULN or | In participants with Gilbert's |
| INR >1.5 | syndrome, doubling of direct |
| | bilirubin should be used for |
| | intervention interruption or |
| | discontinuation decisions rather |
| | than TBL >2 × ULN. |
| ALT or AST >3 × ULN with the appearance of | |
| fatigue, nausea, vomiting, right upper quadrant pain | |
| or tenderness, fever, rash, and/or eosinophilia (>5%) | |
| ALP >3 × ULN, when the source of increased ALP is | |
| the liver | |
| ALP >2.5 × ULN and TBL >2 × ULN | In participants with Gilbert's |
| | syndrome, doubling of direct |
| | bilirubin should be used for |
| | intervention interruption or |
| | discontinuation decisions rather |
| | than TBL >2 × ULN. |
| ALP >2.5 × ULN with the appearance of fatigue, | |
| nausea, vomiting, right upper quadrant pain or | |
| tenderness, fever, rash, and/or eosinophilia (>5%) | |

Resumption of the study intervention can be considered only in consultation with the Lilly-designated medical monitor and only if the liver test results return to baseline and if a self-limited, non-intervention etiology is identified.

### Criteria for Temporary Discontinuation (Withholding) of Study Intervention

Temporary withholding of study intervention is required if the participant meets any of the criteria described in this table.

| Criteria for temporary discontinuation of | |
|---|---|
| study intervention | Next steps |
| Infection Criteria | |
| Serious or opportunistic infections, as | Withhold until resolution of all acute clinical |
| defined in Exclusion Criteria | signs and symptoms, and completion of all |
| | appropriate anti-infective treatment. If |
| | participant is diagnosed with LTBI, see |
| | Section 7.1.3. |
| HBV DNA results that are reported as | Contact sponsor's designated medical |
| positive, or as detecting HBV DNA, but | monitor. |
| HBV DNA is below the level of | Repeat HBV DNA testing as soon as is |
| quantification | feasible. |
| | If HBV DNA is confirmed as positive, |
| | then intervention should be discontinued. |
| Any acute infection or illness | At the discretion of the investigator and |
| | sponsor or its designee, withhold intervention |
| | until resolution of all acute clinical signs and |
| | symptoms, and completion of all appropriate |
| | anti-infective treatment. |
| Hematology Laboratory Criteria | |
| Platelet count <50.0 × 10⁹/L | Contact sponsor's medical monitor |
| WBC <2.0 × 10⁹/L (leukopenia) | designee. |
| Absolute neutrophil count <1.0 × 10⁹/L | Discuss timing of repeat hematology |
| (neutropenia) | laboratory panel and term for withholding |
| Lymphocyte count <0.5 × 10⁹/L | intervention. |
| (lymphopenia) | |

### Criteria for Permanent Discontinuation of Study Intervention

In rare instances, it may be necessary for a participant to permanently discontinue study intervention. If study intervention is permanently discontinued, the participant will remain in the study and follow procedures outlined in the SoA.

### Infections

A participant should be permanently discontinued from study intervention if
the participant develops active TB or HIV infection during the study
the participant is diagnosed with LTBI (regardless if participant qualifies for treatment), or
the participant becomes HBV DNA or HCV RNA positive as described in Section 8.2.7.

Prior to discontinuation of study intervention, the participant is to be referred to, evaluated, and managed by a specialist physician with expertise in evaluation and management of viral hepatitis. The timing of discontinuation from study intervention relative to the initiation of any antiviral treatment for hepatitis is to be based on the recommendation of the consulting specialist physician, in conjunction with the investigator, and aligned with medical guidelines and standard of care.

### Study Assessments and Procedures

Study procedures and their timing are summarized in the SoA.

Immediate safety concerns should be discussed with the sponsor immediately upon occurrence or awareness to determine if the participant should continue or discontinue study intervention.

Adherence to the study design requirements, including those specified in the SoA, is essential and required for study conduct.

All screening evaluations must be completed and reviewed to confirm that potential participants meet all eligibility criteria. The investigator will maintain a screening log to record details of all participants screened and to confirm eligibility or record reasons for screening failure, as applicable.

### Efficacy Assessments

### Primary Efficacy Assessment

The PASI is an investigator-administered, multi-item scale used to measure the severity of psoriasis (EMA 2004).

The PASI is based on the area of coverage and severity of plaque characteristics.

Area of coverage is the extent of body surface involvement in 4 anatomical regions
head and neck
trunk
upper extremities, and
lower extremities.

Plaque characteristics include
the severity of desquamation (scaling)
erythema (redness), and
plaque infiltration (thickness) in each region.

The assessment yields an overall score of 0 for no psoriasis to 72 for the most severe disease (Fredriksson and Pettersson 1978).

The primary efficacy endpoint, PASI 75, represents at least a 75% decrease (improvement) from the baseline PASI score. A PASI 75 response is considered clinically meaningful.

### Secondary Efficacy Assessments

### PASI

Secondary efficacy endpoints, PASI 90 and PASI 100, represent a 90% and 100% improvement from the baseline PASI score.

### Body Surface Area

The percent BSA is an investigator-administered scale used to evaluate the percent involvement of psoriasis on each participant's body surface. It is assessed on a continuous scale from 0% (no involvement) to 100% (full involvement), where 1% corresponds to the size of the participant's hand (including the palm, fingers, and thumb) (National Psoriasis Foundation 2016).

### Static Physician's Global Assessment

The sPGA is an investigator-administered, multi-item scale used in adults. It determines the participant's psoriasis lesions, overall, at a given time point.

Overall lesions are graded for plaque elevation, scaling, and erythema on a range of clear (0), almost clear (1), mild (2), moderate (3), and severe (4) (Cappelleri et al. 2013).

### Psoriasis Scalp Severity Index

The Psoriasis Scalp Severity Index (PSSI) is an investigator-administered, multi-item scale used in adults. It measures the affected scalp area and the severity of clinical symptoms.

The PSSI is a composite score derived from the sum of scores for erythema, induration, and desquamation multiplied by a score for the extent of scalp area involved (range, 0 to 72). The higher scores indicate worse severity (Thaçi et al. 2015).

### Patient-Reported Outcomes

### DLQI

The Dermatology Life Quality Index (DLQI) is a simple, patient-reported, 10-item, validated, quality of life questionnaire. It covers 6 domains
symptoms and feelings
daily activities
leisure
work and school
personal relationships, and
treatment.

The recall period of this scale is over the last week. Response categories include
not at all (score 0)
a little (score 1)
a lot (score 2), and
very much (score 3).

The unanswered (or “not relevant”) responses are scored as 0. Scores range from 0 to 30, with higher scores indicating greater impairment of quality of life.

A DLQI total score of 0 to 1 is considered as having no effect on a participant's health-related quality of life (Hongbo et al. 2005), and a 5-point change from baseline is considered as the minimal clinically important difference threshold (Khilji et al. 2002; Basra et al. 2015).

### Patient's Global Assessment of Psoriasis

The Patient's Global Assessment of Psoriasis (PatGA) is a patient-reported, single-item scale. Patient global assessments allow for an overall evaluation of disease severity or global impact of the disease from the patient's perspective (Perez-Chada et al. 2020). Participants are asked to rank the severity of their psoriasis “today” by selecting a number on a 0 to 5 numeric rating scale, with 0 indicating clear/no psoriasis and 5 indicating severe psoriasis.

### Psoriasis Symptoms Scale

The Psoriasis Symptoms Scale (PSS) is a patient-reported, 8-item scale. It is based on the assessment of
4 symptoms: itch, pain, stinging, and burning
3 signs: redness, scaling, and cracking, and
1 item on the discomfort related to symptoms/signs (Armstrong et al. 2020).

Respondents are asked to answer the questions based on their psoriasis symptoms in the past 24 hours.

Symptoms domain scores range from 0 (no symptoms) to 40 (worst imaginable symptoms).

Signs domain scores range from 0 (no signs) to 30 (worst imaginable signs).

Each of the 8 individual items is scored from 0 to 10, where 0 indicates no symptom/sign and 10 indicates worst imaginable symptom/sign.

The overall severity for each individual symptom or sign ranges from 0 to 10.

### Safety Assessments

Planned time points for all safety assessments are provided in the SoA.

### Vital Signs

Blood pressure, body temperature, and pulse rate will be measured when specified in the SoA and as clinically indicated. Additional vital signs may be measured during study visits if warranted, as determined by the investigator.

Vital signs should be measured after participant has been sitting at least 5 minutes, before obtaining an ECG tracing, and before collection of blood samples for laboratory testing.

### Physical Examinations

### Physical Examination at Screening

The complete physical examination will include assessments of these areas and body systems
Skin
   Head
   Abdomen
   Extremities
Ears, eyes, nose, throat
Lymph nodes
Thyroid palpation
Cardiovascular
Respiratory
Gastrointestinal, and
Neurologic.

Height and weight will also be measured and recorded.

The complete physical examination includes assessment of TB risk factors and symptoms or signs of TB, including an assessment of peripheral lymph nodes (Section 8.2.8).

The complete physical examination excludes pelvic, rectal, and breast examinations, unless clinically indicated.

Symptom-Directed Physical Examination after Screening

These examinations should also include an assessment of TB risk factors and symptoms or signs of TB, including an assessment of peripheral lymph nodes (Section 8.2.8).

### Electrocardiograms

For each participant, a single 12-lead digital ECG will be collected according to the SoA. The ECG should be recorded before collecting any blood. Participants must be supine for approximately 5 to 10 minutes before ECG collection and remain supine but awake during ECG collection.

Electrocardiograms may be obtained at additional times, when deemed clinically necessary. Collection of additional ECGs at a particular time point is allowed to ensure high quality records.

Electrocardiograms will be interpreted by a qualified physician (the investigator or qualified designee) at the study site as soon after the time of ECG collection as possible, and ideally while the participant is still present, to determine whether the participant meets the study entry criteria and for immediate participant management, should any clinically relevant findings be identified.

If a clinically significant finding is identified (including, but not limited to, changes in QT/QTc interval from baseline) after enrollment, the investigator in conjunction with the sponsor will determine if the participant can continue in the study and if any change in participant management is needed.

### Chest Radiography

A posterior-anterior chest X-ray (CXR), interpreted and reported by a radiologist or pulmonologist, will be obtained at screening, as specified in the SoA.

A lateral CXR can also be obtained if, in the opinion of the investigator, a lateral view is indicated.

Participants do not need to have a CXR at screening if, based on the judgment of the investigator, both of the following 2 conditions are met:
the CXR was performed within 3 months before initial screening, and
documentation of the CXR, read by a qualified radiologist or pulmonologist, is sufficient for TB evaluation according to local standard of care.

For each participant, the CXR films, images, or a radiology report must be available to the investigator for review before the participant is randomized. Certain findings from the CXR may be consistent with a condition that excludes a participant from the study (see Section 5.2).

Note: Results of a chest CT scan or other imaging similar to a CXR may be substituted in place of the CXR as described above, in consultation with the sponsor's medical monitor.

### Hepatic Monitoring

### Close Hepatic Monitoring

Laboratory tests (Section 10.2), including ALT, AST, ALP, TBL, direct bilirubin, GGT, and CK, should be repeated within 48 to 72 hours to confirm the abnormality and to determine if it is increasing or decreasing, if one or more of these conditions occur:

| If a participant with baseline | |
|---|---|
| results of . . . | develops the following elevations: |
| ALT or AST <1.5 × ULN | ALT or AST ≥3 × ULN |
| ALP <1.5 × ULN | ALP ≥2 × ULN |
| TBL <1.5 × ULN | TBL ≥2 × ULN (except for participants |
| | with Gilbert's syndrome) |
| ALT or AST ≥1.5 × ULN | ALT or AST ≥2 × baseline |
| ALP ≥1.5 × ULN | ALP ≥2 × baseline |
| TBL ≥1.5 × ULN | TBL ≥1.5 × baseline (except for |
| | participants with Gilbert's syndrome) |

If the abnormality persists or worsens, clinical and laboratory monitoring, and evaluation for possible causes of abnormal liver tests should be initiated by the investigator in consultation with the Lilly-designated medical monitor. At a minimum, this evaluation should include physical examination and a thorough medical history, including symptoms, recent illnesses (for example, heart failure, systemic infection, hypotension, or seizures), recent travel, history of concomitant medications (including over-the-counter), herbal and dietary supplements, history of alcohol drinking and other substance abuse.

Initially, monitoring of symptoms and hepatic biochemical tests should be done at a frequency of 1 to 3 times weekly, based on the participant's clinical condition and hepatic biochemical tests. Subsequently, the frequency of monitoring may be lowered to once every 1 to 2 weeks, if the participant's clinical condition and lab results stabilize. Monitoring of ALT, AST, ALP, and TBL should continue until levels normalize or return to approximate baseline levels.

### Comprehensive Hepatic Evaluation

A comprehensive evaluation should be performed to search for possible causes of liver injury if one or more of these conditions occur:

| If a participant with baseline | |
|---|---|
| results of . . . | develops the following elevations: |
| ALT or AST <1.5 × ULN | ALT or AST ≥3 × ULN with hepatic signs/symptom [symbol] , or |
| | ALT or AST ≥5 × ULN |
| ALP <1.5 × ULN | ALP ≥3 × ULN |
| TBL <1.5 × ULN | TBL ≥2 × ULN (except for participants with Gilbert's |
| | syndrome) |
| ALT or AST ≥1.5 × ULN | ALT or AST ≥2 × baseline with hepatic signs/symptoms [symbol] , or |
| | ALT or AST ≥3 × baseline |
| ALP ≥1.5 × ULN | ALP ≥2 × baseline |
| TBL ≥1.5 × ULN | TBL ≥2 × baseline (except for participants with Gilbert's |
| | syndrome) |

[symbol]

Hepatic signs/symptoms are severe fatigue, nausea, vomiting, right upper quadrant abdominal pain, fever, rash, and/or eosinophilia >5%.

At a minimum, this evaluation should include physical examination and a thorough medical history, as outlined above, as well as tests for PT-INR; tests for viral hepatitis A, B, C, or E; tests for autoimmune hepatitis; and an abdominal imaging study (for example, ultrasound or CT scan).

Based on the participant's history and initial results, further testing should be considered in consultation with the Lilly-designated medical monitor, including tests for hepatitis D virus (HDV), cytomegalovirus (CMV), Epstein-Barr virus (EBV), acetaminophen levels, acetaminophen protein adducts, urine toxicology screen, Wilson's disease, blood alcohol levels, urinary ethyl glucuronide, and blood phosphatidylethanol.

Based on the circumstances and the investigator's assessment of the participant's clinical condition, the investigator should consider referring the participant for a hepatologist or gastroenterologist consultation, magnetic resonance cholangiopancreatography (MRCP), endoscopic retrograde cholangiopancreatography (ERCP), cardiac echocardiogram, or a liver biopsy.

### Additional Hepatic Data Collection (Hepatic Safety CRF) in Study Participants Who Have Abnormal Liver Tests During the Study

Additional hepatic safety data collection in hepatic safety CRFs should be performed in study participants who meet 1 or more of the following 5 conditions:

| | |
|---|---|
| If a participant with baseline | |
| results of . . . | develops the following elevations ... |

1. Elevated serum ALT

| | |
|---|---|
| ALT <1.5 × ULN | ALT to ≥5 × ULN on 2 or more consecutive blood tests |
| ALT ≥1.5 × ULN | ALT ≥3 × baseline on 2 or more consecutive blood tests |

2. Elevated TBL

| | |
|---|---|
| TBL <1.5 × ULN | TBL ≥2 × ULN (except for participants with Gilbert's |
| | syndrome) |
| TBL ≥1.5 × ULN | TBL ≥2 × baseline |

3. Elevated ALP

| | |
|---|---|
| ALP <1.5 × ULN | ALP ≥2 × ULN on 2 or more consecutive blood tests |
| ALP ≥1.5 × ULN | ALP to ≥2 × baseline on 2 or more consecutive blood tests |

4. Hepatic event considered to be a serious adverse event (SAE), or
5. Discontinuation of study intervention due to a hepatic event.
Note:
The interval between the 2 consecutive blood tests should be at least 2 days.

### Hepatitis Testing and Monitoring

### Hepatitis B Virus

Initial testing for HBV infection includes HbsAg and antibody to hepatitis B core antigen (anti-HBc).

| If . . . | Then . . . |
|---|---|
| HbsAg is positive, | the participant is excluded. |
| HbsAg is negative and anti-HBc is negative, | the participant is not excluded. |
| HbsAg is negative and anti-HBc is positive, | further testing for HBV DNA is required. |
| After further screening HBV DNA is positive, | the participant is excluded. |
| After further screening HBV DNA is negative, | the participant is not excluded. |
| | Repeat testing for HBV DNA is required at |
| | least every 3 months during the study, with |
| | temporary withholding or permanent |
| | discontinuation of study intervention if HBV |
| | DNA is positive, as described in Sections |
| | 7.1.2 and 7.1.3. |

### Hepatitis C Virus

Initial testing for HCV infection includes testing for antibodies to HCV (anti-HCV).

| If . . . | Then . . . |
|---|---|
| anti-HCV is positive, | a test for circulating HCV RNA is |
| | required. |
| HCV RNA test is negative, | the participant is not excluded. |
| HCV RNA test is positive, | the participant is excluded. |
| A participant has had an HCV | they are not excluded on the |
| infection and was successfully | basis of HCV as long as HCV |
| treated, defined as a sustained | RNA test is negative at |
| virologic response (HCV RNA by | screening. |
| PCR negative for at least 24 | |
| weeks following treatment | |
| completion) | |

### Tuberculosis Testing and Monitoring

All participants will be assessed for risk factors, symptoms, and signs of TB at screening. Assessments will include
Thorough history to determine the lifetime risk factors for TB infection, for TB progression, and for symptoms and/or signs of active TB, and
Signs of previous or active TB by means of a
   thorough physical examination for signs of active TB, including measurement of body temperature and assessment of peripheral lymph nodes, and
   posterior-anterior chest X-ray (PA CXR) interpreted and reported by radiologist or pulmonologist.

All participants with no history of LTBI or active TB, and no history of positive Mantoux tuberculin skin test (TST) using purified protein derivative (PPD) or positive M tuberculosis interferon gamma release assay (IGRA) must have either a PPD TST or IGRA test.

### Diagnosed LTBI

Participants diagnosed with LTBI are excluded (Section 5.2) unless they have completed appropriate LTBI treatment, per World Health Organization and/or US Centers for Disease Control and Prevention guidelines.

### TB Monitoring

For all participants, monitoring for TB is to be continuous throughout the study. At a minimum, each participant should have the following documented approximately every 3 months:
Thorough history to determine any risk factor for TB infection and for TB progression, symptoms or signs of active TB, and
Thorough physical examination for signs of active TB, including measurement of body temperature and assessment of peripheral lymph nodes.

This table provides details about the TB tests.

| TB test | How to perform the | How to interpret the | |
|---|---|---|---|
| type | test | test | How to retest |
| purified | 1. Inject 0.1 mL of | An induration of 5 | Two-step |
| protein | tuberculin PPD into the | or more mm is considered | testing (that is, repeat |
| derivative | inner surface of the | positive in persons with: | TST 1-3 weeks after |
| tuberculin | forearm. | HIV infection | the first TST) is |
| skin test | The injection | a recent contact | recommended for |
| (PPD TST) | should be made with a | with TB disease | certain participant |
| | tuberculin syringe. | fibrotic changes | groups, including |
| | The TST is an | on chest radiograph | participants: |
| | intradermal injection. | consistent with prior TB | receiving |
| | When placed correctly, | organ transplants | immunosuppressant |
| | the injection should | immunosuppression | treatment |
| | produce a pale elevation | for other reasons (for | with a history |
| | of the skin (a wheal) 6 | example, taking the | of temporally remote |
| | to 10 mm in diameter. | equivalent of >15 mg/day | increased risk of TB |
| | 2. Measure | of prednisone for 1 month | infection |
| | induration at site of | or longer, TNF-α | for whom the |
| | intradermal injection | antagonists) | first test is negative, as |
| | from 48 to 72 hours | An induration of | per local public health |
| | after intradermal | 10 or more mm is | and/or professional |
| | injection. | considered positive in all | medical society |
| | Test must be | other potential clinical | recommendations. |
| | read during this window | trial participants. | |
| | of time. | | |
| | The reaction | | |
| | should be measured in | | |
| | millimeters of | | |
| | induration (palpable, | | |
| | raised, hardened area, or | | |
| | swelling). The reader | | |
| | should not measure | | |
| | erythema (redness). The | | |
| | diameter of the | | |
| | indurated area should be | | |
| | measured across the | | |
| | forearm (perpendicular | | |
| | to the long axis). | | |
| Interferon | Note: Ensure that | Not applicable. | The investigator may |
| gamma | specimen handling, | | discuss retesting with |
| release | transport, timing, and | | the sponsor's |
| assay | laboratory procedures | | designated medical |
| (IGRA) for | meet all requirements | | monitor if: |
| M tuberculosis | per package insert. | | the investigator |
| | | | suspects a false |
| | | | positive IGRA result in |
| | | | a participant with no |
| | | | increased risk of TB |
| | | | infection during |
| | | | lifetime, and |
| | | | there is no |
| | | | evidence of prior or |
| | | | current TB on physical |
| | | | examination and/or on |
| | | | CXR interpreted by |
| | | | radiologist and/or |
| | | | pulmonologist |
| | | | (investigator |
| | | | assessment by history |
| | | | and physical |
| | | | examination. |

Skin Assessment with Fitzpatrick Skin Type Scale

The Fitzpatrick Skin Type is an investigator-administered skin classification scale that classifies the typical response of different types of skin to ultraviolet light. Types range from Type I (very fair; scores 0-6) to Type VI (very dark; scores 35-36) (Fitzpatrick 1988).

### Adverse Events, Serious Adverse Events, and Product Complaints

The definitions of the following events can be found in Section 10.3:
Adverse events (Aes)
Serious adverse events (SAEs), and
Product complaints (PCs).

These events will be reported by the participant (or, when appropriate, by a caregiver, surrogate, or the participant's legally authorized representative).

The investigator and any qualified designees are responsible for detecting, documenting, and recording events that meet these definitions and remain responsible for following up events that are serious, considered related to the study intervention or study procedures, or that caused the participant to discontinue the study intervention or the study.

Care will be taken not to introduce bias when detecting events. Open-ended and non-leading verbal questioning of the participant is the preferred method to inquire about event occurrences.

After the initial report, the investigator is required to proactively follow each participant at subsequent visits/contacts. All SAEs will be followed until resolution, stabilization, the event is otherwise explained, or the participant is lost to follow-up (as defined in Section 7.3). For PCs, the investigator is responsible for ensuring that follow-up includes any supplemental investigations as indicated to elucidate the nature and/or causality. Further information on follow-up procedures is provided in Section 10.3.

### Timing and Mechanism for Collecting Events

This table describes the timing, deadlines, and mechanism for collecting events.

| | | | | | |
|---|---|---|---|---|---|
| | | | Timing for Reporting | | Back-Up |
| | Collection | Collection | to Sponsor or | Mechanism for | Method of |
| Event | Start | Stop | Designee | Reporting | Reporting |

Adverse Event

| | | | | | |
|---|---|---|---|---|---|
| AE | Signing of | Participation | As soon as possible | AE eCRF | N/A |
| | the informed | in study has | upon site awareness | | |
| | consent form | ended | | | |
| | (ICF) | | | | |

Serious Adverse Event

| | | | | | |
|---|---|---|---|---|---|
| SAE and SAE | Signing of | Start of | Within 24 hours of | SAE eCRF | SAE paper |
| updates - prior | the ICF | intervention | awareness | | form |
| to start of study | | | | | |
| intervention | | | | | |
| and deemed | | | | | |
| reasonably | | | | | |
| possibly related | | | | | |
| with study | | | | | |
| procedures | | | | | |
| SAE and SAE | Start of | Participation | Within 24 hours of | SAE eCRF | SAE paper |
| updates - after | intervention | in study has | awareness | | form |
| start of study | | ended | | | |
| intervention | | | | | |
| SAE^{a}- after | After | N/A | Promptly | SAE paper form | N/A |
| participant's | participant's | | | | |
| study | study | | | | |
| participation has | participation | | | | |
| ended and the | has ended | | | | |
| investigator | | | | | |
| becomes aware | | | | | |

Pregnancy

| | | | | | |
|---|---|---|---|---|---|
| Pregnancy in | After the | 4 days after | Within 24 hours | Paper pregnancy | Paper |
| female | start of study | the last dose | (see Section 8.3.2) | form | pregnancy |
| participants and | intervention | of | | | form |
| female partners | | intervention | | | |
| of male | | | | | |
| participants | | | | | |

Product Complaints (PCs)

| | | | | | |
|---|---|---|---|---|---|
| PC associated | Start of study | End of study | Within 24 hours of | PC form | N/A |
| with an SAE or | intervention | intervention | awareness | | |
| might have led | | | | | |
| to an SAE | | | | | |
| PC not | Start of study | End of study | Within 1 business day | PC form | N/A |
| associated with | intervention | intervention | of awareness | | |
| an SAE | | | | | |
| Updated PC | — | — | As soon as possible | Originally | N/A |
| information | | | upon site awareness | completed PC form | |
| | | | | with all changes | |
| | | | | signed and dated by | |
| | | | | the investigator | |
| PC (if | Participation | N/A | Promptly | PC form | |
| investigator | in study has | | | | |
| becomes aware) | ended | | | | |

^{a}SAEs should not be reported unless the investigator deems them to be possibly related to study treatment or study participation.

Adverse Event Monitoring with a Systematic Questionnaire

Nonleading AE collection should occur prior to the collection of the C-SSRS.

If a suicide-related event is discovered during the C-SSRS but was not captured during the nonleading AE collection, sites should not change the AE form.

If an AE is serious or leads to discontinuation, it needs to be included on the AE form and the process for reporting SAEs is followed.

### Infections

Completion of the Infection eCRF page is required for each infection reported as an AE or SAE.

The sponsor will identify infections considered to be opportunistic based on the article by Winthrop et al. (2015) (Section 10.8).

### Pharmacokinetics

Venous blood samples will be collected for measurement of plasma concentrations of Compound I as specified in the SoA.

A maximum of 3 samples may be collected at additional time points during the study if warranted and agreed upon between the investigator and the sponsor.

Instructions for the collection and handling of biological samples will be provided by the sponsor. The actual date and time (24-hour clock time) of each sample will be recorded.

Samples will be used to evaluate the PK of Compound I. Samples collected for analyses may also be used to evaluate safety or efficacy aspects related to concerns arising during or after the study.

Intervention concentration information that may unblind the study will not be reported to investigative sites or blinded personnel.

### Bioanalytical

Samples will be analyzed at a laboratory approved by the sponsor and stored at a facility designated by the sponsor. Concentrations of Compound I will be assayed using a validated bioanalytical method. Analyses of samples collected from placebo-treated participants are not planned.

Sample retention is described in Section 10.1.12. Remaining samples used for PK may be pooled and used for exploratory metabolism or bioanalytical method experiments as deemed appropriate.

### Pharmacodynamics

See Section 10.2, Clinical Laboratory Tests, and the SoA for interleukin-19 (IL-19) sample collection information. Sample retention is described in Section 10.1.12.

### Genetics

A whole-blood sample will be collected for pharmacogenetic analysis where local regulations allow. See Section 10.2, Clinical Laboratory Tests, and the SoA for sample collection information.

See Section 10 S for genetic research, custody, and sample retention information.

### Biomarkers

Biomarker samples will be collected according to the SoA and as detailed in Section 10.2 where local regulations allow.

### Skin Biopsy

Baseline (predose) and postdose skin biopsies will be required of participants and will be collected at the times shown in the SoA. A local anesthetic, such as lidocaine, may be applied and skin punch biopsy, approximately 4 mm in size will be obtained. Detailed instructions for handling the biopsy samples after completing the procedure will be provided by the sponsor.

### Blood and Skin Samples

Samples will be collected to measure proteins and ribonucleic acid (RNA) involved in disease processes, including pathways associated with the disease and mechanisms of action, responses to treatment with Compound I, or for investigation of research methods and validation of diagnostic tools or assays.

Samples will be stored and analysis may be performed for research purposes on the drug target, disease process, pathways associated with disease state, mechanism of action of Compound I, or research methods or in validating diagnostic tools or assay(s) related to psoriasis.

Biomarker research is performed to address questions of relevance to drug disposition, target engagement, pharmacodynamics (PD), mechanism of action, variability of subject response (including safety), and clinical outcome. Sample collection is incorporated into clinical studies to enable examination of these questions through measurement of biomolecules including DNA, RNA, proteins, lipids, and other cellular elements.

All samples will be coded with the participant number. These samples and any data generated can be linked back to the participant only by the investigative site personnel.

Samples will be retained at a facility selected by the sponsor or its designee. The maximum duration of retention is described in Section 10.1.12.

### Statistical Considerations

The statistical analysis plan (SAP) will be finalized prior to the first unblinding, and will include a more technical and detailed description of the statistical analyses described in this section. This section is a summary of the planned statistical analyses for the primary and key secondary endpoints.

### Statistical Hypotheses

The primary objective in this study is to evaluate if Compound I 50 and 125 mg administered QD is superior to placebo in achieving PASI 75 at Week 12 in participants with moderate-to-severe psoriasis, excluding women of childbearing potential.

The null hypothesis to be tested in relation to the primary estimand is Compound I 50 and 125 mg administered QD is not different from placebo in achieving PASI 75 at Week 12. The null hypothesis corresponding to the secondary estimand is defined in the SAP.

### Multiplicity Adjustment

The study is for exploratory, instead of confirmatory purpose and thus none of the hypotheses will be adjusted for multiplicity.

### Analyses Sets

| Population | Description |
|---|---|
| Screened | All participants who signed informed |
| | consent. |
| Intention-to-Treat (ITT) | All randomly assigned participants. |
| | Unless otherwise specified, all efficacy |
| | and health outcomes analyses will be |
| | conducted on this population. |
| Safety | All randomly assigned participants who |
| | received at least 1 dose of study treatment. |
| | Safety analyses will be conducted on this |
| | population. |
| Pharmacokinetic (PK) | All participants randomly assigned to |
| Analysis | study intervention and who take at least 1 |
| | dose of study intervention and have PK |
| | data available. |

### Statistical Analyses

### General Considerations

Statistical analysis of this study will be the responsibility of Lilly or its designee. Unless noted in the SAP, summaries will be provided for the treatment period and for the follow-up period of this study. Analyses will be fully detailed in the SAP.

For the efficacy analysis, the treatment comparison will be conducted separately for both Compound I doses versus placebo.

Any change to the data analysis methods described in the protocol will require an amendment only if it changes a principal feature of the protocol. Any other change to the data analysis methods described in the protocol, and the justification for making the change, will be described in the clinical study report (CSR). Additional exploratory analyses of the data will be conducted as deemed appropriate.

### Baseline Data

For efficacy and patient-reported outcomes (PRO), baseline is defined as the last non-missing assessment recorded on or prior to Visit 2.

Missing baseline values will not be imputed.

Detailed definitions of baseline for safety-related analyses will be described in the SAP.

### Continuous Data

Continuous data will be summarized in terms of the mean, standard deviation, minimum, maximum, median, and number of observations.

### Categorical Data

Categorical data will be summarized as frequency counts and percentages.

### Superiority Tests

Unless otherwise specified, all superiority tests will be 2-sided with alpha of 0.05.

### Primary Endpoint and Estimand Analysis

A composite response estimand will be used to analyze the dichotomous primary endpoint, comparison of the proportion of PASI 75 at Week 12. Comparisons will not include data collected after intercurrent events relevant to study treatment, such as taking prohibited medication, or early discontinuation of study treatment due to AE or lack of efficacy.

Participants who discontinue intervention prior to 12 weeks, who are noncompliant with the concomitant medication rule, or who have missing data for other reasons are defined as non-responders, and a non-responder imputation (NRI) will be used for missing observations.

For primary endpoint assessments and other dichotomous efficacy endpoints, treatment comparisons of Compound I 50 mg QD versus placebo and Compound I 125 mg QD versus placebo will be conducted using a Cochran-Mantel-Haenszel (CMH) test while adjusting for the planned stratification factor on the ITT population. The common risk difference, adjusted odds ratio along with the 95% asymptotic confidence interval, and p-value will be reported.

The alternative estimand and sensitivity analysis for the primary estimand for the primary endpoint will be defined in the SAP.

### Secondary Endpoints and Estimands Analyses

### Continuous Endpoint

A composite estimand will be used to analyze continuous efficacy or PRO endpoints. Comparisons will not include data collected after intercurrent events of taking prohibited medication, or early discontinuation of study intervention due to AE of lack of efficacy. Data collected after these intercurrent events will be imputed as baseline value, which assumes participants with those intercurrent events do not benefit from study treatment.

### Missing Data

Multiple imputation, or non-responder imputation will be used to handle missing data due to reasons other than treatment discontinuation due to AE or lack of efficacy, or a protocol deviation for taking prohibited medication. Details will be defined in the SAP.

Analyses when there are endpoints with more than 1 post-baseline measurements in the double-blind treatment period

This table describes the analysis used to assess treatment effect of Compound I 50 mg versus placebo or Compound I 125 mg versus placebo.

| Statistical Model | Mixed-effects for repeated measures (MMRM) |
|---|---|
| Fixed factors | Treatment |
| | Baseline |
| | Treatment-by-baseline |
| | Stratification factor (yes/no prior exposure to |
| | biologics) |
| Random factor | participant |
| Statistical | Type III sums of squares for the least squares means, |
| comparison | and 95% confidence interval |

Endpoints with only 1 post-baseline measurement in double-blind treatment period

This table describes the analysis used to assess treatment effect of Compound I 50 mg versus placebo or Compound I 125 mg versus placebo.

| Statistical Model | Analysis of covariance (ANCOVA) |
|---|---|
| Fixed factors | Treatment |
| | Stratification factor (yes/no prior exposure to |
| | biologics) |
| | Baseline |
| Statistical | Type III sums of squares for the least squares means, |
| comparison | and 95% confidence interval |

### Dichotomous Endpoint

The estimand used to analyze the dichotomous efficacy endpoint is the same as the primary endpoint as defined in Section 9.3.2.

### Safety Analyses

Safety Population data will be descriptively summarized by treatment group and will include
AEs
SAEs
C-SSRS,
QIDS-SR16,
vital signs, and
laboratory analytes.

Categorical safety measures will be summarized with incidence rates. Continuous safety measures will be summarized as mean change by visit. Exposure to study intervention will be calculated for each participant and summarized by treatment group.

AEs will be coded according to the Medical Dictionary for Regulatory Activities (MedDRA) and summarized by system organ class (SOC), preferred term and severity.

A TEAE is defined as an event that first occurred or worsened in severity after baseline. For events that are gender specific, the denominator and computation of the percentage will include only participants from the given gender.

The number and percentage of participants will be summarized by
TEAEs
TEAEs by maximum severity
TEAEs related to study intervention
discontinuations from study intervention due to an AE
SAEs, or
death.

TEAEs (all, by maximum severity), SAEs including deaths, and AEs that lead to discontinuation from study intervention will be summarized and analyzed by MedDRA SOC and preferred term.

Suicide-related thoughts and behaviors occurring during treatment will be summarized based on responses to the C-SSRS consistent with the C-SSRS Scoring and Data Analysis Guide (Columbia Lighthouse Project WWW).

Details of the safety analyses will be specified in the SAP.

### Pharmacokinetic and Pharmacodynamic Analyses

Compound I plasma concentrations will be illustrated graphically and summarized descriptively. If warranted and based on availability of data, the exposure-response relationship of plasma Compound I concentrations to biomarkers, efficacy and/or safety endpoints may be explored. A model-based approach may be implemented using nonlinear mixed effects modeling (NONMEM) or other appropriate software to estimate PK or PD parameters.

### Subgroup Analyses

Subgroup analyses on the primary or some secondary endpoints may include
Demographic factors
   gender
   age
   geographic region, or
   weight
Prior medication, such as prior exposure to biologic treatments, prior exposure to TNF inhibitors, and
Disease characteristic, such as baseline PASI category.

The details of the subgroup analysis will be included in the SAP.

### Interim Analysis

To support development activities, an interim analysis is planned when at least 30% of participants have completed the Week 12 visit or early discontinued. In addition, an interim analysis is planned when approximately 105 participants have enrolled and completed the Week 12 visit or early discontinued.

Additional interim analyses may be conducted as needed.

An assessment of unblinded interim data will be conducted by an internal assessment committee (IAC) with a limited number of prespecified team members who do not have direct site contact or data entry or validation responsibilities (see Section 10.1.5). Only the IAC is authorized to evaluate unblinded interim efficacy.

The study will not be stopped early for efficacy or futility. No adjustment of type I error will be performed.

Unblinding details are specified in the unblinding plan section of the SAP or in a separate unblinding plan document.

The SAP will describe the planned interim analyses in greater detail.

### Sample Size Determination

Approximately 125 participants will be screened to achieve approximately 105 who are randomly assigned to study intervention. The sample size calculation is based on the primary efficacy estimand and its endpoint PASI 75 at Week 12.

Assuming a PASI 75 response rate of approximately 5% for the placebo group and a 2-sided type-1 error rate of 5%, and using the normal approximation method, the study will have over 90% power under a sample size of 35 per arm to detect a treatment difference of 0.3.

Additional participants may be introduced to evaluate new dose levels based on the availability of new data and planned interim analyses.

### Appendix 2: Clinical Laboratory Tests

The tests detailed in the table below will be performed by the central laboratory or by the local laboratory as specified in the table.

In circumstances where the sponsor approves local laboratory testing in lieu of central laboratory testing (in the table below), the local laboratory must be qualified in accordance with applicable local regulations.

Protocol-specific requirements for inclusion or exclusion of participants are detailed in Section 5 of the protocol.

Additional tests may be performed at any time during the study as determined necessary by the investigator or required by local regulations.

Investigators must document their review of the laboratory safety results.

Laboratory results that could unblind the study will not be reported to investigative sites or other blinded personnel until the study has been unblinded.

| Clinical Laboratory Tests | Comments |
|---|---|
| Hematology | Assayed by Lilly-designated laboratory. |
| Hemoglobin | |
| Hematocrit | |
| Erythrocyte count (RBCs - Red Blood | |
| Cells) | |
| Mean cell volume | |
| Mean cell hemoglobin | |
| Mean cell hemoglobin concentration | |
| Leukocytes (WBCs - White Blood | |
| Cells) | |
| Differential and Absolute Count of: | |
| Neutrophils, segmented | |
| Lymphocytes | |
| Monocytes | |
| Eosinophils | |
| Basophils | |
| Platelets | |
| Cell Morphology (RBC and WBC) | |
| Clinical Chemistry | Assayed by Lilly-designated laboratory. |
| Sodium | |
| Potassium | |
| Chloride | |
| Bicarbonate | |
| Total bilirubin | |
| Direct bilirubin | |
| Alkaline phosphatase (ALP) | |
| Alanine aminotransferase (ALT) | |
| Aspartate aminotransferase (AST) | |
| Gamma-glutamyl transferase (GGT) | |
| Blood urea nitrogen (BUN) | |
| Creatinine | |
| Creatine kinase (CK) | |
| Uric acid | |
| Total protein | |
| Albumin | |
| Calcium | |
| Phosphorus | |
| Glucose | |
| Amylase | |
| Lipase | |
| Cholesterol | |
| Triglycerides | |
| Lipid Panel | Assayed by Lilly-designated laboratory. Participant |
| | should not eat or drink anything except water for 12 |
| | hours before test. |
| High-density lipoprotein (HDL) | |
| Low-density lipoprotein (LDL) | |
| Very-low-density lipoprotein | |
| (VLDL-C) | |
| Urinalysis | Assayed by Lilly-designated laboratory. |
| Specific gravity | |
| pH | |
| Protein | |
| Glucose | |
| Ketones | |
| Bilirubin | |
| Urobilinogen | |
| Blood | |
| Nitrite | |
| Urine leukocyte esterase | |
| Microscopic examination of sediment | |
| Hormones (female) | |
| Follicle-stimulating hormone (FSH) | Assayed by Lilly-designated laboratory. Optional, |
| | as needed to confirm participant's postmenopausal |
| | status. |
| Urine Pregnancy | Local laboratory. |
| Serum Pregnancy | Assayed by Lilly-designated laboratory. |
| Calculations | |
| eGFR (CKD-EPI) | |
| Troponin | Assayed by Lilly-designated laboratory. |
| TB, HIV, and Hepatitis Serology | Assayed by Lilly-designated laboratory. |
| Tuberculosis (TB) testing: | See the protocol (Section 8.2.8) for more |
| | information about TB testing. |
| QuantiFERON-TB Gold test | Assayed by Lilly-designated laboratory. |
| T-SPOT or TST | Evaluated locally. |
| HIV testing | Assayed by Lilly-designated laboratory. |
| Hepatitis C Virus (HCV) testing: | |
| HCV antibody | Assayed by Lilly-designated laboratory. |
| HCV RNA | Assayed by Lilly-designated laboratory. |
| Hepatitis B Virus (HBV) testing: | Assayed by Lilly-designated laboratory. |
| HBV DNA | Performed only for participants who test positive for |
| | anti-HBC. |
| Hepatitis B core antibody (Anti- | |
| HBc) | |
| Hepatitis B surface antigen (HBsAg) | |
| Antibody to hepatitis B core | |
| antibody (anti-HBs) | |
| C-reactive protein, high-sensitivity | |
| (hsCRP) | |
| IL-19 | |
| Pharmacokinetic Samples | Assayed by Lilly-designated laboratory. |
| | Results will not be provided to the investigative |
| | sites. |
| Compound I concentration | |
| Biopsy (skin lesion) | |
| H&E stain (skin thickness) | |
| Immunohistochemistry (IHC) | |
| Gene expression analysis | |
| Genetics Sample | Assayed by Lilly-designated laboratory. |
| | Results will not be provided to the investigative |
| | sites. |
| Exploratory Biomarker Storage | Assayed by Lilly-designated laboratory. |
| Samples | Results will not be provided to the investigative |
| | sites. |
| Serum | |
| Plasma (EDTA) | |
| Whole Blood (Epigenetics) | |
| RNA (PAXGene) | |

### Appendix 6: Liver Safety: Suggested Actions and Follow-Up Assessments

### Hepatic Evaluation Testing

The Lilly-designated central laboratory must complete the analysis of all selected testing except for microbiology testing.

Local testing may be performed in addition to central testing when necessary for immediate participant management.

Results will be reported if a validated test or calculation is available.

| Hematology | Clinical Chemistry |
|---|---|
| Hemoglobin | Total bilirubin |
| Hematocrit | Direct bilirubin |
| Erythrocytes (RBCs - red blood cells) | Alkaline phosphatase (ALP) |
| Leukocytes (WBCs - white blood cells) | Alanine aminotransferase (ALT) |
| Differential: | Aspartate aminotransferase (AST) |
| Neutrophils, segmented | Gamma-glutamyl transferase (GGT) |
| Lymphocytes | Creatine kinase (CK) |
| Monocytes | Other Chemistry |
| Basophils | Acetaminophen |
| Eosinophils | Acetaminophen protein adducts |
| Platelets | Alkaline phosphatase isoenzymes |
| Cell morphology (RBC and WBC) | Ceruloplasmin |
| Coagulation | Copper |
| | Ethyl alcohol (EtOH) |
| Prothrombin time, INR (PT-INR) | Haptoglobin |
| Serology | Immunoglobulin A (IgA) (quantitative) |
| Hepatitis A virus (HAV) testing: | Immunoglobulin G (IgG) (quantitative) |
| HAV total antibody | Immunoglobulin M (IgM) (quantitative) |
| HAV IgM antibody | Phosphatidylethanol (PEth) |
| Hepatis B virus (HBV) testing: | Urine Chemistry |
| Hepatitis B surface antigen (HBsAg) | Drug screen |
| Hepatitis B surface antibody (anti- | Ethyl glucuronide (EtG) |
| HBs) | |
| Hepatitis B core total antibody (anti- | Other Serology |
| HBc) | |
| Hepatitis B core IgM antibody | Anti-nuclear antibody (ANA) |
| Hepatitis B core IgG antibody | Anti-smooth muscle antibody (ASMA) ^{a} |
| HBV DNA ^{b} | Anti-actin antibody ^{c} |
| Hepatis C virus (HCV) testing: | Epstein-Barr virus (EBV) testing: |
| HCV antibody | EBV antibody |
| HCV RNA ^{b} | EBV DNA ^{b} |
| Hepatitis D virus (HDV) testing: | Cytomegalovirus (CMV) testing: |
| HDV antibody | CMV antibody |
| Hepatitis E virus (HEV) testing: | CMV DNA ^{b} |
| HEV IgG antibody | Herpes simplex virus (HSV) testing: |
| HEV IgM antibody | HSV (Type 1 and 2) antibody |
| HEV RNA ^{b} | HSV (Type 1 and 2) DNA ^{b} |
| Microbiology ^{d} | Liver kidney microsomal type 1 (LKM-1) |
| | antibody |
| Culture: | |
| Blood | |
| Urine | |

^{a} Not required if anti-actin antibody is tested.
^{b} Reflex/confirmation dependent on regulatory requirements, testing availability, or both.
^{c} Not required if anti-smooth muscle antibody (ASMA) is tested.
^{d} Assayed ONLY by investigator-designated local laboratory; no central testing available.

### Appendix 7: Permitted and Prohibited Concomitant Medications

### Permitted Concomitant Medications

This section describes concomitant medications and vaccinations allowed in this study.

Other medications may be allowed if they are approved by the sponsor or its designee.

Permitted Concomitant Medications

| Drug Class | Comments |
|---|---|
| Concomitant treatments for psoriasis | |
| Topical steroids and emollients | Class 6 (mild) or 7 (least potent) topical |
| | steroids will be permitted for use limited to |
| | the face, axilla, and/or genitalia as needed. |
| | Emollients may be used on nontarget lesions |
| | (plaque psoriasis lesions that have not been or |
| | will not be biopsied) only if applied using a |
| | stable regimen beginning at least 14 days |
| | prior to baseline (Visit 2/randomization) and |
| | maintained throughout the study. |
| | These topical medications should be avoided |
| | approximately 24 hours prior to visits |
| | requiring the PASI assessment. |
| Additional topical treatments | The following will be allowed, as needed, |
| | throughout the study non-target lesions: |
| | Nonmedicated shampoos |
| | Bath oils, oatmeal bath preparations, and <3% |
| | salicylic acid preparations |
| | These topical treatments should not be used within |
| | approximately 24 hours prior to visits requiring the |
| | PASI assessment. |
| Other concomitant treatments | |
| Cannabinoid products | May be allowed for medical reasons during this |
| | study at the investigator's discretion. |
| Vaccinations | Use of live or live attenuated vaccines is |
| | permitted up to 4 weeks before screening. |
| | A non-live or inactivated vaccine is allowed if |
| | it is received at least 2 weeks before |
| | randomization in the study or after the last |
| | visit. |
| | Inactivated influenza (“Flu”), pneumococcal, |
| | and SARS-CoV-2 vaccines are allowed during |
| | the study. |
| | Note: It is recommended that study intervention |
| | not be administered on the same day as a |
| | SARS-CoV-2 vaccination. |

Abbreviations: PASI = Psoriasis Area and Severity Index; SARS-CoV-2 = severe acute respiratory syndrome coronavirus 2.

### 1.1.1. Prohibited Concomitant Medications and Procedures

This section describes medications prohibited in the study. If a prohibited treatment listed here is required, the study intervention should be permanently discontinued (Section 7.1.3).

Prohibited Concomitant Medications and Procedures

| Drug Class/Procedure | Comments |
|---|---|
| Systemic nonbiologic psoriasis therapy including, | Prohibited within 4 weeks prior to |
| but not limited to: | baseline and during the study. |
| cyclosporine | |
| corticosteroids | |
| methotrexate | |
| oral retinoids | |
| mycophenolate mofetil | |
| thioguanine | |
| hydroxyurea | |
| sirolimus | |
| azathioprine | |
| fumaric acid derivatives | |
| apremilast | |
| 1,25-dihydroxyvitamin D₃ and analogs, or | |
| phototherapy including either | |
| oral and topical PUVA light therapy | |
| ultraviolet B, or | |
| self-treatment with tanning beds or | |
| therapeutic sunbathing. | |
| Topical psoriasis treatments, including but not | Prohibited within 14 days prior to |
| limited to: | baseline and during the study, with |
| moderate/high potency corticosteroids | the exception of the topical |
| anthralin | medications described in |
| calcipotriene | Section 10.7.1. |
| topical vitamin D derivatives | |
| retinoids | |
| tazarotene | |
| pimecrolimus | |
| tacrolimus | |
| topical JAK inhibitors | |
| topical PDE-4 inhibitors | |
| topical AHR inhibitors, or | |
| other nonprescription topical products containing | |
| urea | |
| >3% salicylic acid | |
| alpha- or beta-hydroxyl acids, or | |
| medicated shampoos, for example, those that | |
| contain | |
| >3% salicylic acid | |
| corticosteroids | |
| coal tar, or | |
| vitamin D₃ analogs | |
| Biologics for treatment of immune conditions | Prohibited within 12 weeks prior to |
| | baseline or 5 half-lives prior to |
| | baseline or during the study. |
| Due to DDI risks, concomitant medications | Prohibited within 14 days prior to |
| prohibited in this study include, but are not limited to | baseline and during the study. |
| the following drugs: | |
| Strong CYP3A4 inhibitors | |
| Atazanavir, boceprevir, ceritinib, | |
| clarithromycin, cobicistat, conivaptan, | |
| idelalisib, itraconazole, josamycin, | |
| ketoconazole, lonfarnib, mifepristone, | |
| nefazodone, nelfinavir, posaconazole, | |
| ribociclib, telithromycin, tucatinib, | |
| voriconazole | |
| Ritonavir (by itself, or in combination with | |
| danoprevir, elvitegravir, fosamprenavir, | |
| indinavir, lopinavir, paritaprevir, saquinavir, | |
| tipranavir) | |
| Strong CYP3A4 inducers | |
| Aminoglutethimide, apalutamide, | |
| carbamazepine, enzalutamide, fosphenytoin, | |
| ivosidenib, lumacaftor, mitotane, | |
| phenobarbital, phenytoin, rifabutin, rifampin | |
| (rifampicin), rifapentine, St. John's Wort | |
| Sensitive CYP3A4 substrates with narrow | |
| therapeutic index | |
| Alfentanil, atorvastatin, lovastatin, | |
| midazolam, quetiapine, quinidine, sildenafil, | |
| simvastatin, sirolimus, tacrolimus, triazolam | |
| Transporter P-gp substrates with narrow | |
| therapeutic index | |
| Digoxin | |

Abbreviations: DDI = drug-drug interaction; JAK = Janus Kinase; PUVA = psoralen plus ultraviolet A.

Appendix 8: Examples of Infections that may be Considered Opportunistic

This table provides examples of infections that may be considered opportunistic (Adapted from Winthrop et al. [2015]). This list is not exhaustive.

Bacterial
Bartonellosis (disseminated disease only)
Campylobacteriosis (invasive disease only)
Legionellosis
Listeriosis (invasive disease only)
Nocardiosis
Tuberculosis
Non-tuberculous mycobacterial disease
Salmonellosis (invasive disease only)
Shigellosis (invasive disease only)
Vibriosis (invasive disease due to Vibrio vulnificus)
Viral
BK virus disease including polyomavirus-associated nephropathy
Cytomegalovirus disease
Hepatitis B virus reactivation
Hepatitis C virus progression
Herpes simplex (invasive disease only)
Herpes zoster (any form)
Post-transplant lymphoproliferative disorder (Epstein-Barr virus)
Progressive multifocal leukoencephalopathy (PML), John
Cunningham (JC) virus
Fungal
Aspergillosis (invasive disease only)
Blastomycosis
Candidiasis (invasive disease or oropharyngeal, esophageal.
Not isolated lingual)
Coccidioidomycosis
Cryptococcosis
Histoplasmosis
Paracoccidioides infections
Penicilliosis
Pneumocystosis
Sporotrichosis
Other invasive molds:
Mucormycosis (zygomycosis) (Rhizopus, Mucor, and Lichtheimia)
Scedosporium/Pseudallescheria boydii, and
Fusarium
Parasitic
Leishmaniasis (visceral only)
Strongyloidiasis (hyperinfection syndrome or disseminated disease)
Microsporidiosis
Toxoplasmosis
Trypanosoma cruzi infection (Chagas' disease progression)
(disseminated disease only)
Cryptosporidiosis (chronic disease only)

### Appendix 11: Abbreviations and Definitions

| Term | Definition |
|---|---|
| AE | adverse event |
| blinding | A single-blind study is one in which the investigator and/or his staff are |
| | aware of the treatment but the participant is not, or vice versa, or when |
| | the sponsor is aware of the treatment but the investigator and/his staff |
| | and the participant are not. |
| | A double-blind study is one in which neither the participant nor any of |
| | the investigator or sponsor staff who are involved in the treatment or |
| | clinical evaluation of the participants are aware of the treatment received. |
| BSA | body surface area |
| CIOMS | Council for International Organizations of Medical Sciences |
| complaint | A complaint is any written, electronic, or oral communication that |
| | alleges deficiencies related to the identity, quality, purity, durability, |
| | reliability, safety or effectiveness, or performance of a drug or drug |
| | delivery system. |
| compliance | Adherence to all study-related, good clinical practice (GCP), and |
| | applicable regulatory requirements. |
| CRF | case report form |
| C-SSRS | Columbia Suicide-Severity Rating Scale |
| CSR | clinical study report |
| CT scan | Computed tomography scan |
| CXR | chest X-ray |
| DLQI | Dermatology Life Quality Index |
| EDC | electronic data capture |
| enroll | The act of assigning a participant to a treatment. Participants who are |
| | enrolled in the study are those who have been assigned to a treatment. |
| enter | Participants entered into a study are those who sign the informed consent |
| | form directly or through their legally acceptable representatives. |
| GCP | good clinical practice |
| HBV | hepatitis B virus |
| HBsAg | hepatitis B surface antigen |
| HCV | hepatitis C virus |
| HIV | human immunodeficiency virus |
| IAC | Internal Assessment Committee |
| IB | Investigator's Brochure |
| ICF | informed consent form |
| ICH | International Council for Harmonisation |
| IGRA | interferon gamma release assay |
| IL-19 | interleukin-19 |
| informed consent | A process by which a participant voluntarily confirms his or her |
| | willingness to participate in a particular study, after having been |
| | informed of all aspects of the study that are relevant to the participant's |
| | decision to participate. Informed consent is documented by means of a |
| | written, signed and dated informed consent form. |
| INR | International normalized ratio |
| interim analysis | An interim analysis is an analysis of clinical study data, separated into |
| | treatment groups, that is conducted before the final reporting database is |
| | created/locked. |
| intervention | A pharmaceutical form of an active ingredient or placebo being tested or |
| | used as a reference in a clinical trial, including products already on the |
| | market when used or assembled (formulated or packaged) in a way |
| | different from the authorized form, or marketed products used for an |
| | unauthorized indication, or marketed products used to gain further |
| | information about the authorized form. |
| ITT | intention to treat: The principle that asserts that the effect of a treatment |
| | policy can be best assessed by evaluating on the basis of the intention to |
| | treat a participant (that is, the planned treatment regimen) rather than the |
| | actual treatment given. It has the consequence that participant allocated |
| | to a treatment group should be followed up, assessed, and analyzed as |
| | members of that group irrespective of their compliance to the planned |
| | course of treatment. |
| IWRS | interactive web-response system |
| LTBI | latent TB infection |
| Medical monitor | Individual responsible for the medical conduct of the study. |
| | Responsibilities of the medical monitor may be performed by a clinical |
| | research physician, clinical research scientist, global safety physician, or |
| | other medical officer designated by the sponsor. |
| participant | Equivalent to CDISC term “subject”: an individual who participates in a |
| | clinical trial, either as recipient of an investigational medicinal product or |
| | as a control. |
| PASI | Psoriasis Area and Severity Index |
| PC | product complaint |
| PK/PD | pharmacokinetics/pharmacodynamics |
| PPD | purified protein derivative |
| PRO | patient-reported outcomes/electronic patient-reported outcomes |
| PSSI | Psoriasis Scalp Severity Index |
| QD | once daily |
| QIDS | Quick Inventory of Depressive Symptomatology |
| RIPK1 | receptor-interacting protein kinase 1 |
| SAD | single ascending dose |
| SAE | serious adverse event |
| SAP | statistical analysis plan |
| screen | The act of determining if an individual meets minimum requirements to |
| | become part of a pool of potential candidates for participation in a |
| | clinical study. |
| SIB | suicidal ideation and behavior |
| SOC | system organ class |
| sPGA | Static Physician's Global Assessment |
| TB | tuberculosis |
| TEAE | Treatment-emergent adverse event: An untoward medical occurrence that |
| | emerges during a defined treatment period, having been absent |
| | pretreatment, or worsens relative to the pretreatment state, and does not |
| | necessarily have to have a causal relationship with this treatment. |
| TNF | tumor necrosis factor |
| TST | tuberculin skin test |

What is claimed:

1. A method of treating a patient having an autoimmune or inflammatory disease, comprising administering to the patient a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, or a pharmaceutically acceptable salt thereof, wherein the compound or the pharmaceutically acceptable salt is administered at a dose of about 12.5 mg to about 125 mg free base equivalent of said compound per dose.

2. The method of claim 1, wherein the autoimmune or inflammatory disease is a disease modulated by receptor-interacting protein (RIP) kinase 1.

3. The method of claim 1, wherein the autoimmune or inflammatory disease is selected from the group consisting of rheumatoid arthritis, psoriasis, ulcerative colitis, Crohn's disease, cutaneous lupus erythematosus, lupus nephritis, systemic lupus erythematosus, and atopic dermatitis.

4. The method of claim 1, wherein the administering is carried out with an amorphous form of the compound.

5. The method of claim 4, wherein the amorphous form of the compound is part of a solid dispersion that comprises a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC).

6. The method of claim 1, wherein the dose is about 12.5 mg, 18.75 mg, 25 mg, 31.25 mg, 37.5 mg, 43.75 mg, 50 mg, 56.25 mg, 62.5 mg, 68.75 mg, 75 mg, 81.25 mg, 87.5 mg, 93.75 mg, 100 mg, 106.25 mg, 112.5 mg, 118.75 mg, or 125 mg free base equivalent of said compound per dose.

7. The method of claim 1, wherein the dose is about 50 mg to about 75 mg free base equivalent of said compound per dose.

8. The method of claim 1, wherein the dose is about 25 mg to about 50 mg free base equivalent of said compound per dose.

9. The method of claim 1, wherein the dose is about 75 mg to about 100 mg free base equivalent of said compound per dose.

10. The method of claim 1, wherein the dose is about 100 mg to about 125 mg free base equivalent of said compound per dose.

11. The method of claim 1, wherein the maximum daily dose is about 25 mg, 50 mg, 75 mg, 100 mg, or 125 mg free base equivalent of said compound.

12. A method of treating a patient having an autoimmune or inflammatory disease, comprising administering to the patient an amorphous form of compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide, wherein the compound or the pharmaceutically acceptable salt is administered at a dose of about 12.5 mg to about 125 mg free base equivalent of said compound per dose, and wherein the dose is administered once per day.

13. The method of claim 12, wherein administering of the dose is via oral administration.

14. The method of claim 12, wherein the autoimmune or inflammatory disease is rheumatoid arthritis, psoriasis, or ulcerative colitis.

15. The method of claim 12, wherein the autoimmune or inflammatory disease is rheumatoid arthritis, and the compound is administered at a dose of about 25 mg to about 125 mg per dose.

16. The method of claim 12, wherein the autoimmune or inflammatory disease is psoriasis, and the compound is administered at a dose of about 25 mg to about 125 mg per dose.

17. The method of claim 12, wherein the autoimmune or inflammatory disease is ulcerative colitis.

18. A pharmaceutical composition, comprising:
a compound of(S)-5-benzyl-N-(7-(3-hydroxy-3-methylbut-1-yn-1-yl)-5-methyl-4-oxo-2,3,4,5-tetrahydrobenzo[b][1,4]oxazepin-3-yl)-1H-1,2,4-triazole-3-carboxamide in an amorphous form, and
a polymer selected from the group consisting of hydroxypropyl methylcellulose acetate succinate (HPMC-AS), polyvinylpyrrolidone/vinyl acetate co-polymer, 6:4, Povidone K30, and hydroxypropyl methylcellulose (HPMC),
wherein a weight ratio (w/w) of the compound to the polymer is about 1:4 to about 1.2:1.

19. The pharmaceutical composition of claim 18, wherein the polymer comprises hydroxypropyl methylcellulose acetate succinate (HPMC-AS).

20. The pharmaceutical composition of claim 19, wherein a weight ratio (w/w) of free base equivalent of the compound and the hydroxypropyl methylcellulose acetate succinate is about 1:3 to about 1:1.
